(19) United States
(12) Patent Application Publication
(10) Pub. No.: US 2026/0327326 A1
(43) Pub. Date: Oct. 22, 2026

(54) T CELL BINDING COMPOSITIONS AND METHODS TARGETING CD20 AND BCMA

(71) Applicant: ASTRAZENECA AB, Södertälje (SE)

(72) Inventors: Raymond Joseph MONIZ, Wilmington, DE (US); Sahver Tilbe CREIGH-PULATMEN, Cambridge (GB); Zohra MERAZGA, Wilmington, DE (US); Naresh KUMAR, Wilmington, DE (US); Karin Cristina GOLAN, Wilmington, DE (US); Christopher Owen LLOYD, Cambridge (GB)

(21) Appl. No.: 19/642,012

(22) Filed: Apr. 8, 2026

Related U.S. Application Data
(60) Provisional application No. 63/785,798, filed on Apr. 9, 2025.

(51) Int. Cl. C07K 16/28; A61K 39/00; A61P 35/00; A61P 37/02

(52) U.S. Cl. CPC C07K 16/2878; A61P 35/00; A61P 37/02; C07K 16/2809; C07K 16/2815; C07K 16/2887; A61K 2039/505; A61K 2039/54; C07K 2317/31; C07K 2317/522; C07K 2317/524; C07K 2317/526; C07K 2317/565; C07K 2317/622; C07K 2317/75

(57) ABSTRACT

The disclosure generally relates to binding proteins that comprise antigen binding sites to CD20 and BCMA, a T cell receptor binding site, and a T cell co-stimulatory molecule binding site. The disclosure also relates to pharmaceutical compositions comprising such binding proteins, nucleic acid molecules encoding such binding proteins, and vectors comprising such nucleic acid molecules. The disclosure further relates to methods of treating a disorder or condition associated with CD20 and/or BCMA using such binding proteins and pharmaceutical compositions, binding proteins and pharmaceutical compositions for use in the treatment of a disorder or condition, and the use of such binding proteins and pharmaceutical compositions for the manufacture of a medicament for treating a disorder or condition associated with CD20 and/or BCMA.

### CROSS-REFERENCE TO RELATED APPLICATIONS

This application claims the benefit of U.S. Provisional Patent Application No. 63/785,798, filed on Apr. 9, 2025, titled “T Cell Binding Compositions and Methods Targeting CD20 and BCMA,” the entire contents of which are hereby incorporated by reference.

### REFERENCE TO SEQUENCE LISTING SUBMITTED ELECTRONICALLY

This application is accompanied by a computer-readable sequence listing submitted electronically herewith in an XML file titled, “CD20BCMATITAN-100-US02-NP.xml,” created on Apr. 1, 2026 and having a file size of 159,207 bytes, the entire contents of which are hereby incorporated by reference.

### FIELD OF THE DISCLOSURE

The disclosure generally relates to binding proteins that comprise antigen binding sites to CD20 and BCMA, a T cell receptor binding site, and a T cell co-stimulatory molecule binding site. The disclosure also relates to pharmaceutical compositions comprising such binding proteins, nucleic acid molecules encoding such binding proteins, and vectors comprising such nucleic acid molecules. The disclosure further relates to methods of treating a disorder or condition using such binding proteins and pharmaceutical compositions, binding proteins and pharmaceutical compositions for use in the treatment of a disorder or condition, and the use of such binding proteins and pharmaceutical compositions for the manufacture of a medicament for treating a disorder or condition.

### BACKGROUND OF THE DISCLOSURE

Recruitment of T cell cytotoxic activity to destroy tumor cells is a worthwhile but complicated treatment strategy for cancer. The development of CD3-based bispecific T cell engagers (TCEs) as cancer therapeutics has been ongoing for the past 30 years. TCEs simultaneously bind a tumor associated antigen (TAA) and cluster of differentiation 3 (CD3) on a T cell to form a T cell receptor (TCR)-independent artificial immune synapse, circumventing human leukocyte antigen (HLA) restriction, and inducing T cell activation and cytolysis of the tumor cell.

The first generation of TCEs were simple bispecific T cell engagers (BiTEs), composed of two tandem single-chain variable fragments (scFvs) including a strong CD3-binding arm and a TAA binding domain. To date, there is only one Food and Drug Administration-approved BiTE, blinatumomab, which targets CD3 (using the Orthoclone OKT3 antibody) and cluster of differentiation 19 (CD19). The strong in vitro cytolytic activity observed during the development of BiTEs created excitement around their potential use for treating cancer. However, the unanticipated high cytokine release syndrome (CRS) observed in the clinic somewhat tempered that excitement. Teachey et al., “Cytokine release syndrome after blinatumomab treatment related to abnormal macrophage activation and ameliorated with cytokine-directed therapy,” Blood 121:5154-57 (2013). Another observed disadvantage of BiTE formats is that they exhibit very short half-life and have poor manufacturability. Ellerman, “Bispecific T cell engagers: Towards understanding variables influencing the in vitro potency and tumor selectivity and their modulation to enhance their efficacy and safety,” Methods 154:102-17 (2019).

The second generation of TCEs include a fragment crystallizable (Fc) domain which can be modified to confer half-life extension and mutations to eliminate Fc receptor (FcR) binding, and present improved manufacturability. Vafa et al., “Perspective: Designing T cell Engagers With Better Therapeutic Windows,” Front. Oncol. 10:446 (2020). Nevertheless, those molecules still include high affinity-CD3 binding domains, linking to induction of neurotoxicity and CRS in the clinic. More recent efforts have been focused on developing CD3 binding domains with reduced affinity with the hope to maintain potent T cell activation while significantly reducing associated cytokine release. Trinklein et al., “Efficient tumor killing and minimal cytokine release with novel T cell agonist bispecific antibodies,” MAbs 11:639-52 (2019).

Importantly, both CD3-based BiTEs and novel immunoglobulin G (IgG)-format TCEs bind and activate both cluster of differentiation 4 (CD4) and cluster of differentiation 8 (CD8) T cells, potentially engaging unfavorable T cells such as regulatory T cells (Treg), which have been shown to potentially decrease the cytolytic activity of CD8 T cells. Duell et al., “Frequency of regulatory T cells determines the outcome of the T cell-engaging antibody blinatumomab in patients with B-precursor ALL,” Leukemia 31:2181-90 (2017).

While T cell engager molecules offer promise, the therapeutic approach has faced challenges to date. There is a need in the art for improved T-cell binding proteins with increased activity and reduced off-target effects. Effective treatments for B cell-related malignancies and conditions have focused on specific cellular targets, such as CD20 (Cluster of Differentiation 20) and BCMA (B Cell Maturation Antigen).

BCMA is a tumor necrosis family receptor (TNFR) member expressed on cells of the B-cell lineage including antibody secreting cells (plamsa cells and plasmablasts) (Laabi et al., Nucleic Acids Research, 22 (7): 1147-1154 (1994)). BCMA is involved in mediating the survival of plasma cells for maintaining long-term humoral immunity. The expression of BCMA has been linked to a number of cancers, autoimmune disorders, and infectious diseases.

CD20 is a phosphate glycoprotein transmembrane protein predominantly expressed on the surface of B cells and is involved in various stages of B cell development, maturation and activation. CD20 is predominantly found in various B cell malignancies, including Non-Hodgkin lymphoma (NHL), chronic lymphocytic leukemia (CLL) and autoimmune disorders. Rituximab is monoclonal antibody that targets CD20 used to treat autoimmune diseases and types of cancer (B-cell malignancies). By targeting CD20, these therapies facilitate the destruction of malignant B cells can enhance immunological responses.

Unfortunately, patients sometimes become resistant to CD20 or BCMA therapies, or the therapies prove to be too toxic. There is a need for alternative and/or improved therapies to treat cancer and inflammatory and/or autoimmune disorders.

### SUMMARY OF THE DISCLOSURE

In a first aspect the disclosure provides a binding protein comprising four polypeptide chains that form two different tumor-associated antigen (TAA) binding sites, a T cell receptor binding site, and a T cell co-stimulatory molecule binding site; wherein one tumor-associated antigen (TAA) binding site binds to B-cell maturation antigen (BCMA) and the other tumor-associated antigen (TAA) binding site binds to CD20.

In a second aspect the disclosure provides a binding protein wherein the first polypeptide chain has a structure represented by the formula:

V_{L1}—C_{L1a}
a second polypeptide chain has a structure represented by the formula:

V_{L2}—C_{L1b}
a third polypeptide chain has a structure represented by the formula:

V_{H1}—C_{H1a}—V_{HHa}—Fcₐ
and a fourth polypeptide chain which has a structure represented by the formula:

V_{H2}—C_{H1b}—V_{HHb}—Fc_{b}
wherein the V_{L1} domain of the first polypeptide and the V_{H1} domain of the third polypeptide form the first of the two TAA binding sites, and the V_{L2} domain of the second polypeptide and the V_{H2} domain of the fourth polypeptide form the second of the two TAA binding sites, and wherein:
   V_{L1} is an immunoglobulin light chain variable domain and V_{H1} is an immunoglobulin heavy chain variable domain that together form a TAA binding domain that specifically binds to BCMA;
   V_{L2} is an immunoglobulin light chain variable domain and V_{H2} is an immunoglobulin heavy chain variable domain that together form a TAA binding domain that specifically binds to CD20;
   C_{L1a} and C_{L1b} are each an immunoglobulin light chain constant domain;
   C_{H1a} and C_{H1b} are each an immunoglobulin CH1 heavy chain constant domain;
   V_{HHa} is a single chain variable domain that specifically binds a T cell receptor (TCR);
   V_{HHb} is a single chain variable domain that specifically binds a T cell co-stimulatory molecule;
   Fcₐ comprises a C_{H2a} and a C_{H3a} immunoglobulin heavy chain constant domain, wherein the C_{H2a} domain is a first CH2 immunoglobulin heavy chain constant domain and the C_{H3a} domain is a first CH3 immunoglobulin heavy chain constant domain; and
   Fc_{b} comprises a C_{H2b} and a C_{H3b} immunoglobulin heavy chain constant domain wherein the C_{H2b} domain is a second CH2 immunoglobulin heavy chain constant domain and the C_{H3b} domain is a second CH3 immunoglobulin heavy chain constant domain.

In a third aspect the disclosure provides a binding protein, wherein the first polypeptide chain has a structure represented by the formula:

V_{L1}—C_{L1a}
a second polypeptide chain has a structure represented by the formula:

V_{L2}—C_{L1b}
a third polypeptide chain has a structure represented by the formula:

V_{H2}—C_{H1a}—V_{HHa}—Fcₐ
and a fourth polypeptide chain which has a structure represented by the formula:

V_{H1}—C_{H1b}—V_{HHb}—Fc_{b}
wherein the V_{L1} domain of the first polypeptide and the V_{H1} domain of the fourth polypeptide form the first of the two TAA binding sites, and the V_{L2} domain of the second polypeptide and V_{H2} domain of the third polypeptide form the second of the two TAA binding sites, and wherein:
   V_{L1} is an immunoglobulin light chain variable domain and V_{H1} is an immunoglobulin heavy chain variable domain that together form a TAA binding domain that specifically binds to BCMA;
   V_{L2} is an immunoglobulin light chain variable domain and V_{H2} is an immunoglobulin heavy chain variable domain that together form a TAA binding domain that specifically binds to CD20;
   C_{L1a} and C_{L1b} are each an immunoglobulin light chain constant domain;
   C_{H1a} and C_{H1b} are each an immunoglobulin CH1 heavy chain constant domain;
   V_{HHa} is a single chain variable domain that specifically binds a T cell receptor (TCR);
   V_{HHb} is a single chain variable domain that specifically binds a T cell co-stimulatory molecule;
   Fcₐ comprises a C_{H2a} and a C_{H3a} immunoglobulin heavy chain constant domain, wherein the C_{H2a} domain is a first CH2 immunoglobulin heavy chain constant domain and the C_{H3a} domain is a first CH3 immunoglobulin heavy chain constant domain; and
   Fc_{b} comprises a C_{H2b} and a C_{H3b} immunoglobulin heavy chain constant domain wherein the C_{H2b} domain is a second CH2 immunoglobulin heavy chain constant domain and the C_{H3b} domain is a second CH3 immunoglobulin heavy chain constant domain.

In a fourth aspect the disclosure provides a binding protein wherein the first polypeptide chain has a structure represented by the formula:

V_{L2}—C_{L1a}
a second polypeptide chain has a structure represented by the formula:

V_{L1}—C_{L1b}
a third polypeptide chain has a structure represented by the formula:

V_{H2}—C_{H1a}—V_{HHa}—Fcₐ
and a fourth polypeptide chain which has a structure represented by the formula:

V_{H1}—C_{H1b}—V_{HHb}—Fc_{b}
wherein the V_{L1} domain of the first polypeptide and the V_{H1} domain of the fourth polypeptide form the first of the two TAA binding sites, and the V_{L2} domain of the second polypeptide and V_{H2} domain of the third polypeptide form the second of the two TAA binding sites, and wherein:
   V_{L1} is an immunoglobulin light chain variable domain and V_{H1} is an immunoglobulin heavy chain variable domain that together form a TAA binding domain that specifically binds to BCMA;
   V_{L2} is an immunoglobulin light chain variable domain and V_{H2} is an immunoglobulin heavy chain variable domain that together form a TAA binding domain that specifically binds to CD20;
   C_{L1a} and C_{L1b} are each an immunoglobulin light chain constant domain;
   C_{H1a} and C_{H1b} are each an immunoglobulin CH1 heavy chain constant domain;
   V_{HHa} is a single chain variable domain that specifically binds a T cell receptor (TCR);
   V_{HHb} is a single chain variable domain that specifically binds a T cell co-stimulatory molecule;
   Fcₐ comprises a C_{H2a} and a C_{H3a} immunoglobulin heavy chain constant domain, wherein the C_{H2a} domain is a first CH2 immunoglobulin heavy chain constant domain and the C_{H3a} domain is a first CH3 immunoglobulin heavy chain constant domain; and
   Fc_{b} comprises a C_{H2b} and a C_{H3b} immunoglobulin heavy chain constant domain wherein the C_{H2b} domain is a second CH2 immunoglobulin heavy chain constant domain and the C_{H3b} domain is a second CH3 immunoglobulin heavy chain constant domain.

In a fifth aspect the disclosure provides a binding protein wherein the first polypeptide chain has a structure represented by the formula:

V_{L2}—C_{L1a}
a second polypeptide chain has a structure represented by the formula:

V_{L1}—C_{L1b}

a third polypeptide chain has a structure represented by the formula:

V_{H2}—C_{H1a}—V_{HHa}—Fcₐ

and a fourth polypeptide chain which has a structure represented by the formula:

V_{H1}—C_{H1b}—V_{HHb}—Fc_{b}

wherein the V_{L1} domain of the second polypeptide and the V_{H1} domain of the fourth polypeptide form the first of the two TAA binding sites, and the V_{L2} domain of the first polypeptide and V_{H2} domain of the third polypeptide form the second of the two TAA binding sites, and wherein:
V_{L1} is an immunoglobulin light chain variable domain and V_{H1} is an immunoglobulin heavy chain variable domain that together form a TAA binding domain that specifically binds to BCMA;
V_{L2} is an immunoglobulin light chain variable domain and V_{H2} is an immunoglobulin heavy chain variable domain that together form a TAA binding domain that specifically binds to CD20;
C_{L1a} and C_{L1b} are each an immunoglobulin light chain constant domain;
C_{H1a} and C_{H1b} are each an immunoglobulin CH1 heavy chain constant domain;
V_{HHa} is a single chain variable domain that specifically binds a T cell receptor (TCR);
V_{HHb} is a single chain variable domain that specifically binds a T cell co-stimulatory molecule;
Fcₐ comprises a C_{H2a} and a C_{H3a} immunoglobulin heavy chain constant domain, wherein the C_{H2a} domain is a first CH2 immunoglobulin heavy chain constant domain and the C_{H3a} domain is a first CH3 immunoglobulin heavy chain constant domain; and
Fc_{b} comprises a C_{H2b} and a C_{H3b} immunoglobulin heavy chain constant domain wherein the C_{H2b} domain is a second CH2 immunoglobulin heavy chain constant domain and the C_{H3b} domain is a second CH3 immunoglobulin heavy chain constant domain.

In a sixth aspect, the disclosure provides a method of treating cancer in a subject in need thereof, comprising administering a therapeutically effective amount of the binding protein or pharmaceutical composition of the disclosure.

In a seventh aspect, the disclosure provides a method of treating cancer, or a method of treating an inflammatory and/or autoimmune disorder in a subject in need thereof, comprising administering a therapeutically effective amount of the binding protein or pharmaceutical composition of the disclosure.

### BRIEF DESCRIPTION OF THE FIGURES

The accompanying figures are included to provide a further understanding of the methods and compositions of the disclosure and are incorporated in and constitute a part of this disclosure. The figures illustrate one or more aspects of the disclosure and together with the description serve to explain the principles and operation of the disclosure.

FIG. 1 illustrates the structure of exemplary monospecific and bispecific TCEs described herein: BCMA:BCMA TPP-47646 (FIG. 1A), CD20: CD20 TPP-47645 (FIG. 1B), BCMA:CD20 TPP-47648 (FIG. 1C) and CD20:BCMA TPP-47651 (FIG. 1D).

FIG. 2 illustrates a more detailed structure of BCMA:CD20 TPP-47648 and CD20:BCMA TPP-47651.

FIG. 3 shows the schematics and amino acid sequences for the various T-cell engagers exemplified herein: TPP-47648 (BCMA:CD20) (FIG. 3A), TPP-47651 (CD20:BCMA) (FIG. 3B), TPP-47646 (BCMA:BCMA) (FIG. 3C), TPP-47645 (CD20: CD20) (FIG. 3D).

FIG. 4 shows the in vitro cytolytic activity of TPP-47646, TPP-47645, TPP-47648 (BCMA:CD20), and TPP-47651 (CD20:BCMA) with BCMA+CD20− (FIG. 4A) or BCMA-CD20+ (FIG. 4B) cell lines incubated with total Pan T cells at an E:T ratio of 2.5:1. Each line represents a single donor. FIG. 4C shows the EC₅₀.

FIG. 5 shows the in vitro corresponding T cell activation profiles as % CD4 and CD8 T cells expressing CD25 following in vitro cytolysis assays with BCMA+CD20− (FIG. 5A) or BCMA-CD20+ (FIG. 5B) cell lines incubated with total Pan T cells at an E:T ratio of 2.5:1 and a TCE selected from TPP-47646, TPP-47645, TPP-47648 (BCMA:CD20), and TPP-47651 (CD20:BCMA).

FIG. 6 shows depletion of B cells (FIG. 6A) and selective activation of CD8+ cytotoxic T cells (FIG. 6B) in healthy donor PBMCs treated with TPP-47648.

FIG. 7 shows depletion of plasmablasts/plasma cells (FIG. 7A) and selective activation of CD8+ cytotoxic T cells (FIG. 7B) in healthy donor PBMCs treated with TPP-47648.

FIG. 8. shows in vitro cytokine profile induced by TPP-47648 for IFN-γ, IL6, IL17A, TNFa in a BCMA+CD20− Cell line (NCI-H929).

FIG. 9 shows the in vitro cytokine profile induced by TPP-47648 (TPP48) for IFN-γ, IL-10, IL-6, IL-17A, TNFa in healthy donor PBMCs.

FIG. 10 shows the depletion of B cells in the spleen (FIG. 10A), bone marrow (FIG. 10B) and blood (FIG. 10C) of human NGS-CD34+ engrafted mice treated with with a single dose of either vehicle, isotype control LM-1909 or 0.04 mg/kg, 0.15 mg/kg, 0.65 mg/kg 2.5 mg/kg or 10 mg/kg of TPP-47468. FIG. 10D shows the depletion of plasmablasts and plasma cells in the spleen.

FIG. 11 shows the in vivo depletion of plasmablasts in the bone marrow (FIG. 11A) and spleen (FIG. 11B), and the serum levels of IgG (FIG. 11C) of the xenogeneic model of graft versus host disease (XenoGvHD) treated with either phosphate buffered saline (PBS), 0.3 mg/kg isotype control, or 0.3 mg/kg, 0.1 mg/kg, 0.03 mg/kg, 0.01 mg/kg, 0.003 mg/kg TPP-47468.

FIG. 12 shows the in vivo depletion of memory B cells in Cynomolgus Macaque with administration of TPP-47648 (FIG. 12A) or TPP-47651 (FIG. 12B).

FIG. 13 shows the reduction of tumor size in mice injected with NCI-H929 (BCMA+CD20-isolate) following treatment with BCMA:CD20 and CD20:BCMA TCE compared to PBS.

### DETAILED DESCRIPTION OF THE DISCLOSURE

The disclosure generally relates to binding proteins that comprise antigen binding sites to CD20 and BCMA, a T cell receptor binding site, and a T cell co-stimulatory molecule binding site. The disclosure also relates to pharmaceutical compositions comprising such binding proteins, nucleic acid molecules encoding such binding proteins, and vectors comprising such nucleic acid molecules. The disclosure further relates to methods of treating a disorder or condition using such binding proteins and pharmaceutical compositions, binding proteins and pharmaceutical compositions for use in the treatment of a disorder or condition, and the use of such binding proteins and pharmaceutical compositions for the manufacture of a medicament for treating a disorder or condition.

Percentages disclosed herein can vary in amount by ±10%, 20%, or 30% from values disclosed and remain within the scope of the contemplated disclosure.

As used herein, ranges and amounts can be expressed as “about” a particular value or range. The term “about” also includes the exact amount. For example, “about 5%” means “about 5%” and also “5%.” The term “about” can also refer to ±10% of a given value or range of values. Therefore, about 5% also means 4.5%-5.5%, for example.

As utilized in accordance with the present disclosure, unless otherwise indicated, all technical and scientific terms shall be understood to have the same meaning as commonly understood by one of ordinary skill in the art.

The term “binding protein,” as used herein, refers to a non-naturally occurring (or recombinant) molecule which comprises multiple polypeptide chains that form at least one antigen binding site.

A “recombinant” molecule is one that has been prepared, expressed, created, or isolated by recombinant DNA technology means.

The term “antibody,” as used herein, refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The variable domain may be subjected to further protein engineering to humanize the framework regions if the antibody was derived from a non-human source. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between CH1 and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4. Throughout this specification, unless otherwise indicated, amino acid residues in variable domain sequences and variable domain regions within full antigen binding sequences are numbered according to the Kabat numbering convention.

The term “native Fc,” as used herein, refers to a molecule comprising the sequence of a non-antigen binding fragment resulting from digestion of an antibody or produced by other means, whether in monomeric or multimeric form, and can contain the hinge region. The original immunoglobulin source of the native Fc is preferably of human origin and can be any of the immunoglobulins. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3 and IgG4). One example of a native Fc is a disulfide-bonded dimer resulting from papain digestion of an IgG. The term “native Fc,” as used herein, is generic to the monomeric, dimeric, and multimeric forms.

The term “Fc variant,” as used herein, refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed or mutated to produce an Fc variant to alter certain residues that provide structural features or biological activity that are not required for the binding proteins of the disclosure. Thus, the term “Fc variant” comprises a molecule or sequence that lacks one or more native Fc sites or residues, or in which one or more Fc sites or residues has been modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “Fc,” as used herein, encompasses native Fc and Fc variants as defined above. As with Fc variants and native Fc molecules, the term “Fc” includes molecules in monomeric or multimeric form, whether digested from whole antibody or produced by other means.

Binding proteins encompassed by this disclosure can be of or be derived from any type (e.g., IgG, IgE, IgM, IgD, IgA, and IgY), class (e.g., IgG1, IgG2, IgG3, IgG4, IgA1, and IgA2), or subclass of immunoglobulin molecule.

The terms “knob,” “hole,” and “knob in hole” as used herein refer to a previously developed system to direct the correct pairing of heavy chains from differing antibodies. A “knob” and a “hole” are engineered into the two heavy chains of differing antibodies in order to promote correct pairing. The “knob in hole” approach is an effective way to produce bispecific antibodies by driving heterodimerization with mutations in the CH3 domain of each half antibody.

The term “antigen” or “target antigen,” as used herein, refers to a molecule or a portion of a molecule that is capable of being recognized by and bound by the antigen binding portion of the binding proteins of the disclosure. The target antigen is capable of being used in an animal to produce antibodies capable of binding to an epitope of that antigen. A target antigen may have one or more epitopes. With respect to each target antigen recognized by the antigen binding portion of the binding protein, is capable of competing with an intact antibody that recognizes the target antigen.

The term “antigen binding site,” as used herein, refers to a site created on the surface of a binding protein of the disclosure where an antigen or an epitope on an antigen is bound.

The term “linker,” as used herein, refers to one or more amino acid residues inserted between domains of the binding protein of the disclosure. For example, a linker may be inserted between domains, at the sequence level. The precise location of a domain transition can be determined by locating peptide stretches that do not form secondary structural elements such as beta-sheets or alpha-helices as demonstrated by experimental data or as can be assumed by techniques of modeling or secondary structure prediction. Linkers may or may not be needed depending on the where the stop and start residues of protein fusions are chosen because often natural linkers are found between immunoglobulin domains.

The skilled person will appreciate that, upon production of the binding proteins of the disclosure, post-translational modifications may occur. For example, this may include cleavage of certain leader sequences, the addition or various sugar moieties in various glycosylation patterns, non-enzymatic glycation, deamidation, oxidation, disulfide bond scrambling and other cysteine variants such as free sulfhydryls, recemized disulfides, thioethers and trisulfide bonds, isomerisation, C-terminal lysine clipping and N-terminal glutamine cyclisation. The present disclosure encompasses binding proteins that have been subjected to, or have undergone, one or more post translational modifications.

The use of “−” in the context of a molecular formula represents a direct or indirect link between two components, for example a direct covalent bond or an indirect link for example a peptide chain, such as a linker or hinge.

As used herein, the term “polynucleotide” includes a singular nucleic acid as well as multiple nucleic acids, and refers to an isolated nucleic acid molecule or construct, e.g., messenger RNA (mRNA) or plasmid DNA (pDNA). The term “nucleic acid” includes any nucleic acid type, such as DNA or RNA.

As used herein, the term “vector” can refer to a nucleic acid molecule as introduced into a host cell, thereby producing a transformed host cell. A vector can include nucleic acid sequences that allows the vector to replicate in a host cell, such as an origin of replication. A vector can also include one or more selectable marker gene and other genetic elements known in the art. Specific types of vector envisioned here can be associated with or incorporated into viruses to facilitate cell transformation.

In one aspect, there is provided a vector comprising the isolated nucleic acid molecule of any one of SEQ ID NOs: 118-121. In another aspect, there is provided a vector comprising the isolated nucleic acid molecule of any one of SEQ ID NOs: 122-125.

As used herein, the terms “treat,” “treatment,” or “treatment of” refer to reducing disease pathology, reducing or eliminating disease symptoms, promoting increased survival rates, and/or reducing discomfort. For example, treating can refer to the ability of a therapy to reduce disease symptoms, signs, or causes when administered to a subject. Treating also refers to mitigating or decreasing at least one clinical symptom and/or inhibition or delay in the progression of the condition and/or prevention or delay of the onset of a disease or illness.

The terms “administration” or “administering,” as used herein, refer to providing, contacting, and/or delivering a binding protein by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

As used herein, the terms “subject,” “individual,” or “patient,” refer to a human, for whom diagnosis, prognosis, or therapy is desired.

As used herein, the term an “effective amount” or a “therapeutically effective amount” of an administered therapeutic substance, such as a binding protein, is an amount sufficient to carry out a specifically stated or intended purpose, such as treating or treatment of cancer. An “effective amount” can be determined empirically in a routine manner in relation to the stated purpose.

The term “pharmaceutical composition,” as used herein, refer to a compound or composition capable of inducing a desired therapeutic effect when properly administered to a subject. In some aspects, the disclosure provides a pharmaceutical composition comprising a pharmaceutically acceptable carrier and a therapeutically effective amount of binding proteins of the disclosure. The terms “pharmaceutically acceptable carrier” or “physiologically acceptable carrier,” as used herein, refer to one or more formulation materials suitable for accomplishing or enhancing the delivery of one or more binding proteins of the disclosure.

In some aspects, the binding proteins disclosed herein may be formulated with a pharmaceutically acceptable carrier, excipient, or stabilizer, as pharmaceutical compositions. In certain aspects, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required. In some aspects, there is provided a pharmaceutical composition comprising the binding protein of the disclosure and a pharmaceutically acceptable carrier.

In some aspects provided herein is a binding protein comprising two tumor-associated antigen (TAA) binding sites, wherein the tumor associated antigens (TAA) are B-cell maturation antigen (BCMA) and cluster of differentiation 20 (CD20). CD20 is a transmembrane protein involved in Ca⁺⁺ channeling, B cell activation, and proliferation.

CD20 is a membrane-embedded surface molecule which plays a role in the development and differentiation of B cells into plasma cells.

B-cell maturation antigen (BCMA), also known as tumor necrosis factor receptor superfamily member 17 (TNFRSF17), is a member of the TNF-receptor superfamily, which is preferentially expressed in mature B lymphocytesand is important for B cell development, proliferation, and survival. BCMA is expressed on antibody secreting cells (plasmablasts and plasma cells).

In some aspects provided herein is a binding protein comprising four polypeptide chains that form two different tumor-associated antigen (TAA) binding sites, a T cell receptor binding site, and a T cell co-stimulatory molecule binding site; wherein one tumor-associated antigen (TAA) binding site binds to B-cell maturation antigen (BCMA) and the other tumor-associated antigen (TAA) binding site binds to CD20. Targeting both CD20 and BCMA proteins expressed by B cells allows for B cell lineage depletion i.e. depletion of B cells expressing CD20, and antibody secreting cells (plasma and plasmablasts) expressing BCMA.

In another aspect, there is provided a binding protein wherein the first polypeptide chain has a structure represented by the formula:

V_{L1}—C_{L1a}
a second polypeptide chain has a structure represented by the formula:

V_{L2}—C_{L1b}
a third polypeptide chain has a structure represented by the formula:

V_{H1}—C_{H1a}—V_{HHa}—Fcₐ
and a fourth polypeptide chain which has a structure represented by the formula:

V_{H2}—C_{H1b}—V_{HHb}—Fc_{b}
wherein the V_{L1} domain of the first polypeptide and the V_{H1} domain of the third polypeptide form the first of the two TAA binding sites, and the V_{L2} domain of the second polypeptide and the V_{H2} domain of the fourth polypeptide form the second of the two TAA binding sites, and wherein:
   V_{L1} is an immunoglobulin light chain variable domain and V_{H1} is an immunoglobulin heavy chain variable domain that together form a TAA binding domain that specifically binds to BCMA;
   V_{L2} is an immunoglobulin light chain variable domain and V_{H2} is an immunoglobulin heavy chain variable domain that together form a TAA binding domain that specifically binds to CD20;
   C_{L1a} and C_{L1b} are each an immunoglobulin light chain constant domain;
   C_{H1a} and C_{H1b} are each an immunoglobulin CH1 heavy chain constant domain;
   V_{HHa} is a single chain variable domain that specifically binds a T cell receptor (TCR);
   V_{HHb} is a single chain variable domain that specifically binds a T cell co-stimulatory molecule;
   Fcₐ comprises a CH₂ₐ and a CH₃ₐ immunoglobulin heavy chain constant domain, wherein the CH2ₐ domain is a first CH2 immunoglobulin heavy chain constant domain and the CH3a domain is a first CH3 immunoglobulin heavy chain constant domain; and;
   Fc_{b} comprises a C_{H2b} and a C_{H3b} immunoglobulin heavy chain constant domain wherein the C_{H2b} domain is a second CH2 immunoglobulin heavy chain constant domain and the C_{H3b} domain is a second CH3 immunoglobulin heavy chain constant domain.

In another aspect, there is provided a binding protein, wherein the first polypeptide chain has a structure represented by the formula:

V_{L1}—C_{L1a}
a second polypeptide chain has a structure represented by the formula:

V_{L2}—C_{L1b}
a third polypeptide chain has a structure represented by the formula:

V_{H2}—C_{H1a}—V_{HHa}—Fcₐ
and a fourth polypeptide chain which has a structure represented by the formula:

V_{H1}—C_{H1b}—V_{HHb}—Fc_{b}
wherein the V_{L1} domain of the first polypeptide and the V_{H1} domain of the fourth polypeptide form the first of the two TAA binding sites, and the V_{L2} domain of the second polypeptide and V_{H2} domain of the third polypeptide form the second of the two TAA binding sites, and wherein:
   V_{L1} is an immunoglobulin light chain variable domain and V_{H1} is an immunoglobulin heavy chain variable domain that together form a TAA binding domain that specifically binds to BCMA;
   V_{L2} is an immunoglobulin light chain variable domain and V_{H2} is an immunoglobulin heavy chain variable domain that together form a TAA binding domain that specifically binds to CD20;
   C_{L1a} and C_{L1b} are each an immunoglobulin light chain constant domain;
   C_{H1a} and C_{H1b} are each an immunoglobulin CH1 heavy chain constant domain;
   V_{HHa} is a single chain variable domain that specifically binds a T cell receptor (TCR);
   V_{HHb} is a single chain variable domain that specifically binds a T cell co-stimulatory molecule;
   Fcₐ comprises a CH₂ₐ and a CH₃ₐ immunoglobulin heavy chain constant domain, wherein the CH2ₐ domain is a first CH2 immunoglobulin heavy chain constant domain and the CH3ₐ domain is a first CH3 immunoglobulin heavy chain constant domain; and;
   Fc_{b} comprises a CH2_{b} and a CH3_{b} immunoglobulin heavy chain constant domain wherein the CH2_{b} domain is a second CH2 immunoglobulin heavy chain constant domain and the CH3_{b} domain is a second CH3 immunoglobulin heavy chain constant domain.

In another aspect, there is provided a binding protein, wherein the first polypeptide chain has a structure represented by the formula:

V_{L2}—C_{L1a}
a second polypeptide chain has a structure represented by the formula:

V_{L1}—C_{L1b}
a third polypeptide chain has a structure represented by the formula:

V_{H2}—C_{H1a}—V_{HHa}—Fcₐ
and a fourth polypeptide chain which has a structure represented by the formula:

V_{H1}—C_{H1b}—V_{HHb}—Fc_{b}
wherein the V_{L1} domain of the first polypeptide and the V_{H1} domain of the fourth polypeptide form the first of the two TAA binding sites, and the V_{L2} domain of the second polypeptide and V_{H2} domain of the third polypeptide form the second of the two TAA binding sites, and wherein:
   V_{L1} is an immunoglobulin light chain variable domain and V_{H1} is an immunoglobulin heavy chain variable domain that together form a TAA binding domain that specifically binds to BCMA;
   V_{L2} is an immunoglobulin light chain variable domain and V_{H2} is an immunoglobulin heavy chain variable domain that together form a TAA binding domain that specifically binds to CD20;
   C_{L1a} and C_{L1b} are each an immunoglobulin light chain constant domain;
   C_{H1a} and C_{H1b} are each an immunoglobulin CH1 heavy chain constant domain;
   V_{HHa} is a single chain variable domain that specifically binds a T cell receptor (TCR);
   V_{HHb} is a single chain variable domain that specifically binds a T cell co-stimulatory molecule;
   Fcₐ comprises a CH₂ₐ and a CH₃ₐ immunoglobulin heavy chain constant domain, wherein the CH2ₐ domain is a first CH2 immunoglobulin heavy chain constant domain and the CH3ₐ domain is a first CH3 immunoglobulin heavy chain constant domain; and;
   Fc_{b} comprises a CH2_{b} and a CH3_{b} immunoglobulin heavy chain constant domain wherein the CH2_{b} domain is a second CH2 immunoglobulin heavy chain constant domain and the CH3_{b} domain is a second CH3 immunoglobulin heavy chain constant domain.

In another aspect, there is provided a binding protein, wherein the first polypeptide chain has a structure represented by the formula:

V_{L2}—C_{L1a}
a second polypeptide chain has a structure represented by the formula:

V_{L1}—C_{L1b}
a third polypeptide chain has a structure represented by the formula:

V_{H2}—C_{H1a}—V_{HHa}—Fcₐ
and a fourth polypeptide chain which has a structure represented by the formula:

V_{H1}—C_{H1b}—V_{HHb}—Fc_{b}
wherein the V_{L1} domain of the second polypeptide and the V_{H1} domain of the fourth polypeptide form the first of the two TAA binding sites, and the V_{L2} domain of the first polypeptide and V_{H2} domain of the third polypeptide form the second of the two TAA binding sites, and wherein:
   V_{L1} is an immunoglobulin light chain variable domain and V_{H1} is an immunoglobulin heavy chain variable domain that together form a TAA binding domain that specifically binds to BCMA;
   V_{L2} is an immunoglobulin light chain variable domain and V_{H2} is an immunoglobulin heavy chain variable domain that together form a TAA binding domain that specifically binds to CD20;
   C_{L1a} and C_{L1b} are each an immunoglobulin light chain constant domain;
   C_{H1a} and C_{H1b} are each an immunoglobulin CH1 heavy chain constant domain;
   V_{HHa} is a single chain variable domain that specifically binds a T cell receptor (TCR);
   V_{HHb} is a single chain variable domain that specifically binds a T cell co-stimulatory molecule;
   Fcₐ comprises a CH₂ₐ and a CH₃ₐ immunoglobulin heavy chain constant domain, wherein the CH2a domain is a first CH2 immunoglobulin heavy chain constant domain and the CH3a domain is a first CH3 immunoglobulin heavy chain constant domain; and;
   Fc_{b} comprises a CH2_{b} and a CH3_{b} immunoglobulin heavy chain constant domain wherein the CH2_{b} domain is a second CH2 immunoglobulin heavy chain constant domain and the CH3_{b} domain is a second CH3 immunoglobulin heavy chain constant domain.

In some aspects, the binding protein comprises a linker. The identity and sequence of amino acid residues in the linker may vary depending on the type of secondary structural element necessary to be achieved. For example, glycine, serine, and alanine are best for linkers having maximum flexibility. Some combination of glycine, proline, threonine, and serine are useful if a more rigid and extended linker is necessary. Any amino acid residue may be considered as a linker in combination with one or more other amino acid residues, which may be the same as or different as the first amino acid residue, to construct larger peptide linkers as necessary depending on the desired properties. In another aspect, there is provided a binding protein, further comprising L₁, a linker positioned between C_{H1a} and V_{HHa} on the third polypeptide chain, and/or L₂, a linker positioned between V_{HHa} and Fcₐ on the third polypeptide chain, wherein L₁ and L₂ are each independently a linker.

In another aspect, there is provided a binding protein, further comprising L₃, a linker positioned between C_{H1b} and V_{HHb} on the fourth polypeptide chain, and/or L₄, a linker positioned between V_{HHb} and Fc_{b} on the fourth polypeptide chain, wherein L₃ and L₄ are each independently a linker.

In another aspect, there is provided a binding protein, wherein L₁, L₂, L₃, and/or L₄ each independently comprise one or more repeats of the amino acid sequence of SEQ ID NO: 12 and/or SEQ ID NO: 13.

In another aspect, there is provided a binding protein, further comprising H₁, an immunoglobulin hinge region positioned between C_{H1a} and V_{HHa} on the third polypeptide chain, and/or H₂, an immunoglobulin hinge region positioned between V_{HHa} and Fcₐ on the third polypeptide chain, wherein H₁ and H₂ are each independently an immunoglobulin hinge region. In some embodiments, H₁ comprises SEQ ID NO: 28, SEQ ID NO: 29, SEQ ID NO: 30, SEQ ID NO: 59, SEQ ID NO: 60 or SEQ ID NO: 116, and H₂ comprises SEQ ID NO: 28, SEQ ID NO: 29, SEQ ID NO: 30, SEQ ID NO: 59 or SEQ ID NO: 60.

In another aspect, there is provided a binding protein, further comprising H₃, an immunoglobulin hinge region positioned between C_{H1b} and V_{HHb} on the fourth polypeptide chain, and/or H₄, an immunoglobulin hinge region positioned between V_{HHb} and the Fc_{b} on the fourth polypeptide chain, wherein H₃ and H₄ are each independently an immunoglobulin hinge region. In some embodiments, H₃ comprises SEQ ID NO: 28, SEQ ID NO: 29, SEQ ID NO: 30, SEQ ID NO: 59, SEQ ID NO: 60 or SEQ ID NO: 116, and H₄ comprises SEQ ID NO: 28, SEQ ID NO: 29, SEQ ID NO: 30, SEQ ID NO: 59 or SEQ ID NO: 60.

In another aspect, there is provided a binding protein, wherein the first polypeptide chain has a structure represented by the formula

V_{L1}—C_{L1a}
a second polypeptide chain has a structure polypeptide chain represented by the formula:

V_{L2}—C_{L1b}
a third polypeptide chain has a structure represented by the formula:

V_{H1}—C_{H1a}—H₁-L₁-V_{HHa}—H₂-L₂-Fcₐ or

V_{H1}—C_{H1a}—H₁-L₁-V_{HHa}-L₂-H₂-Fcₐ
and a fourth polypeptide chain has a structure represented by the formula:

V_{H2}—C_{H1b}—H₃-L₃-V_{HHb}—H₄-L₄-Fc_{b} or

V_{H2}—C_{H1b}—H₃-L₃-V_{HHb}-L₄-H₄—Fc_{b}.

In another aspect, there is provided a binding protein, wherein the first polypeptide chain has a structure represented by the formula

V_{L2}—C_{L1a}
a second polypeptide chain has a structure polypeptide chain represented by the formula:

V_{L1}—C_{L1b}
a third polypeptide chain has a structure represented by the formula:

V_{H2}—C_{H1a}—H₁-L₁-V_{HHa}—H₂-L₂-Fcₐ or

VH₂-C_{H1a}—H₁-L₁-V_{HHa}-L₂-H₂-Fcₐ
and a fourth polypeptide chain has a structure represented by the formula:

V_{H1}—C_{H1b}—H₃-L₃-V_{HHb}—H₄-L₄-Fc_{b} or

V_{H1}—C_{H1b}—H₃-L₃-V_{HHb}-L₄-H₄—Fc_{b}.

In another aspect, there is provided a binding protein, wherein the binding protein activates T cells only when bound to a tumor associated antigen at one or both of the tumor-associated antigen binding sites.

The binding proteins of the disclosure comprise a T cell receptor (TCR) binding site (V_{HHa}). The TCR comprises a heterodimer including the highly variable alpha (α) and beta (β) chains. The multicomponent complex of the TCR comprises the CD3 co-receptor, which plays a significant role in activating T cells. In one aspect, there is provided a binding protein comprising a single chain variable domain that specifically binds a T cell receptor (V_{HHa}), wherein V_{HHa} comprises a heavy chain CDR1 (HCDR1), a heavy chain CDR2 (HCDR2) and a heavy chain CDR3 (HCDR3), comprising the amino acid sequences of SEQ ID NO: 14, SEQ ID NO: 15 and SEQ ID NO: 16, respectively. In some embodiments, V_{HHa} comprises a heavy chain CDR1 (HCDR1), a heavy chain CDR2 (HCDR2) and a heavy chain CDR3 (HCDR3) comprising the amino acid sequences of SEQ ID NO: 37, SEQ ID NO: 15 and SEQ ID NO: 16, respectively. In some embodiments, V_{HHa} comprises the amino acid sequence at least 90%, at least 91% at least 92%, at least 93%, at least 94%, at least 95%, at least 96%, at least 97%, at least 98%, or at least 99% identical to any one of SEQ ID NOs: 17-23, or any one of SEQ ID NOs: 17-23. In some embodiments, V_{HHa} comprises a heavy chain CDR1 (HCDR1), a heavy chain CDR2 (HCDR2) and a heavy chain CDR3 (HCDR3), comprising the amino acid sequences of SEQ ID NO: 109, SEQ ID NO: 110, and SEQ ID NO: 111 respectively. In other embodiments, V_{HHa} comprises the amino acid sequence at least 90%, at least 91% at least 92%, at least 93%, at least 94%, at least 95%, at least 96%, at least 97%, at least 98%, or at least 99% identical to SEQ ID NO: 108, or SEQ ID NO: 108.

In some aspects provided herein is a binding protein comprising one T cell costimulatory molecule binding site. A co-stimulatory molecule comprises a co-stimulatory domain capable of potentiating or modulating the response of immune effector cells. Co-stimulatory domains can include sequences, for example, from one or more of CD3zeta (or CD3z), CD28, CD137 (4-1BB), OX-40, ICOS, CD27, GITR, CD2, IL-2Rβ and MyD88/CD40. In some aspects, the T cell costimulatory molecule is CD137 (4-1BB). In some aspects, the T cell costimulatory molecule is CD8 and the single chain variable domain that specifically binds CD8 is V_{HHb}. In some embodiments, V_{HHb} comprises a heavy chain CDR1 (HCDR1), a heavy chain CDR2 (HCDR2) and a heavy chain CDR3 (HCDR3) comprising the amino acid sequences of SEQ ID NO: 24, SEQ ID NO: 25 and SEQ ID NO: 26, respectively. In some embodiments, V_{HHb} comprises a heavy chain CDR1 (HCDR1), a heavy chain CDR2 (HCDR2) and a heavy chain CDR3 (HCDR3) comprising the amino acid sequences of SEQ ID NO: 105, SEQ ID NO: 106 and SEQ ID NO: 107, respectively. In other embodiments, V_{HHb} comprises an amino acid sequence at least 90%, at least 91% at least 92%, at least 93%, at least 94%, at least 95%, at least 96%, at least 97%, at least 98%, or at least 99% identical to SEQ ID NO: 104, or SEQ ID NO: 104.

In some aspects, the Fcₐ and/or Fc_{b} is an IgG isotype. In some embodiments, Fcₐ and/or the Fc_{b} is an IgG1 isotype. In other embodiments, Fcₐ and the Fc_{b} are both IgG1 isotypes.

In some aspects, C_{H3a} and C_{H3b} are both CH3 immunoglobulin heavy chain constant domains. In some aspects, C_{H3a} and C_{H3b} in each of Fcₐ and Fc_{b} comprise modifications to facilitate heterodimerization of Fcₐ and Fc_{b}. In various non-limiting embodiments, the modifications are substitutions to generate a knob in one of Fcₐ and Fc_{b} and a hole in the other of Fcₐ and Fc_{b}. In one nonlimiting embodiment the knob is a substitution to tryptophan at position 366, and the hole is a substitution of one or more of the following: i) a substitution to valine at position 407; ii) a substitution to serine at position 366; and iii) a substitution to alanine at position 368. In another non-limiting embodiment, the Fcₐ or the Fc_{b} containing the knob further comprises a cysteine at position 354 and/or the Fcₐ or the Fc_{b} containing the hole comprises a cysteine at position 349 and wherein the numbering is according to the Eu index.

In some aspects, C_{H2a} and C_{H2b} are both CH2 immunoglobulin heavy chain constant domains. In some aspects, C_{H2a} and C_{H2b} immunoglobulin heavy chain constant domains each comprise the following substitutions: E233P/L234V/L235A/G236del/S267K and wherein the numbering is according to the Eu index. In other aspects, either the CH3a immunoglobulin heavy chain constant domain or C_{H3b} immunoglobulin heavy chain constant domain comprises a H435R and Y436F substitution, and wherein the numbering is according to the Eu index.

In various non-limiting embodiments Fcₐ comprises the amino acid sequence of SEQ ID NO: 31 or SEQ ID NO: 32. In various other non-limiting embodiments Fc_{b} comprises the amino acid sequence of SEQ ID NO: 33.

In some aspects, the third polypeptide chain comprises either SEQ ID NO: 34 or SEQ ID NO: 35 and the fourth polypeptide chain comprises SEQ ID NO: 36.

In some aspects, the third polypeptide chain comprises SEQ ID NO: 135 and the fourth polypeptide chain comprises SEQ ID NO: 136. In some aspects, there is provided a binding protein, wherein the TAA binding domain that specifically binds to CD20 comprises a heavy chain CDR1 (HCDR1), a heavy chain CDR2 (HCDR2), a heavy chain CDR3 (HCDR3), a light chain CDR1 (LCDR1), a light chain CDR2 (LCDR2) and a light chain CDR3 (LCDR3), comprising the amino acid sequences of SEQ ID NO: 1, SEQ ID NO: 2, SEQ ID NO: 3, SEQ ID NO: 4, SEQ ID NO: 5 and SEQ ID NO: 6, respectively. In some embodiments, the TAA binding domain comprises a V_{H2} that is at least 90%, at least 91% at least 92%, at least 93%, at least 94%, at least 95%, at least 96%, at least 97%, at least 98%, or at least 99% identical to SEQ ID NO: 7; and a V_{L2} domain that is at least 90%, at least 91% at least 92%, at least 93%, at least 94%, at least 95%, at least 96%, at least 97%, at least 98%, or at least 99% identical to SEQ ID NO: 8; or comprises a V_{H2} domain according to SEQ ID NO: 7 and a V_{L2} domain according to SEQ ID NO: 8.

In another aspect, there is provided a binding protein wherein the TAA binding domain that specially binds to BCMA comprises a heavy chain CDR1 (HCDR1), a heavy chain CDR2 (HCDR2), a heavy chain CDR3 (HCDR3), a light chain CDR1 (LCDR1), a light chain CDR2 (LCDR2) and a light chain CDR3 (LCDR3), comprising the amino acid sequences of SEQ ID NO: 76, SEQ ID NO: 77, SEQ ID NO: 78, SEQ ID NO: 79, SEQ ID NO: 80 and SEQ ID NO: 81, respectively. In some embodiments, there is provided a binding protein, wherein the TAA binding domain comprises a V_{H1} that is at least 90%, at least 91% at least 92%, at least 93%, at least 94%, at least 95%, at least 96%, at least 97%, at least 98%, or at least 99% identical to SEQ ID NO: 82; and a V_{L1} domain that is at least 90%, at least 91% at least 92%, at least 93%, at least 94%, at least 95%, at least 96%, at least 97%, at least 98%, or at least 99% identical to SEQ ID NO: 83; or comprises a V_{H1} domain according to SEQ ID NO: 82 and a V_{L1} domain according to SEQ ID NO: 83.

In some aspects, there is provided a binding protein comprising the amino acid sequences of SEQ ID NO: 10, SEQ ID NO: 84, SEQ ID NO: 11 and SEQ ID NO: 86. In some embodiments, the first polypeptide chain comprises the amino acid sequence of SEQ ID: 84, the second polypeptide chain comprises the amino acid sequence of SEQ ID: 10, the third polypeptide chain comprises the amino acid sequence of SEQ ID: 86, and the fourth polypeptide chain comprises the amino acid sequence of SEQ ID: 11. In some embodiments, the binding protein is a BCMA:CD20 TCE that has a first polypeptide chain having the amino acid sequence of SEQ ID: 84, a second polypeptide chain having the amino acid sequence of SEQ ID: 10, a third polypeptide chain having the amino acid sequence of SEQ ID: 86, and a fourth polypeptide chain having the amino acid sequence of SEQ ID: 11; this embodiment is referred to herein as TPP-47648 or TPP48.

In some aspects, there is provided a binding protein comprising the amino acid sequences of SEQ ID NO: 89, SEQ ID NO: 88, SEQ ID NO: 90 and SEQ ID NO: 91. In some embodiments, the first polypeptide chain comprises the amino acid sequence of SEQ ID: 89, the second polypeptide chain comprises the amino acid sequence of SEQ ID: 88, the third polypeptide chain comprises the amino acid sequence of SEQ ID: 91, and the fourth polypeptide chain comprises the amino acid sequence of SEQ ID: 90. In some embodiments, the binding protein is a CD20:BCMA TCE that has a first polypeptide chain having the amino acid sequence of SEQ ID: 89, a second polypeptide chain having the amino acid sequence of SEQ ID: 88, a third polypeptide chain having the amino acid sequence of SEQ ID: 91, and a fourth polypeptide chain having the amino acid sequence of SEQ ID: 90; this embodiment is referred to herein as TPP-47651.

In one-non-limiting embodiment, the binding protein activates T cells only when bound to a tumor associated antigen at one or both of the tumor associated antigen binding sites. Some aspects described herein provides a binding protein of claim 1, wherein the first polypeptide chain has a structure represented by the formula: V_{L1}—C_{L1a} a second polypeptide chain has a structure represented by the formula: V_{L2}—C_{L1b} a third polypeptide chain has a structure represented by the formula: V_{H1}—C_{H1a}—V_{HHa}—Fcₐ and a fourth polypeptide chain which has a structure represented by the formula: V_{H2}—C_{H1b}—V_{HHb}—Fc_{b} wherein the V_{L1} domain of the first polypeptide and the V_{H1} domain of the third polypeptide form the first of the two TAA binding sites, and the V_{L2} domain of the second polypeptide and the V_{H2} domain of the fourth polypeptide form the second of the two TAA binding sites, and wherein: V_{L1} is an immunoglobulin light chain variable domain and V_{H1} is an immunoglobulin heavy chain variable domain that together form a TAA binding domain that specifically binds to BCMA; V_{L2} is an immunoglobulin light chain variable domain and V_{H2} is an immunoglobulin heavy chain variable domain that together form a TAA binding domain that specifically binds to CD20; C_{L1a} and C_{L1b} are each an immunoglobulin light chain constant domain; C_{H1a} and C_{H1b} are each an immunoglobulin CH1 heavy chain constant domain; V_{HHa} is a single chain variable domain that specifically binds a T cell receptor; V_{HHb} is a single chain variable domain that specifically binds a T cell co-stimulatory molecule; Fcₐ comprises a CH₂ₐ and a CH₃ₐ immunoglobulin heavy chain constant domain, wherein the CH2ₐ domain is a first CH2 immunoglobulin heavy chain constant domain and the CH3ₐ domain is a first CH3 immunoglobulin heavy chain constant domain; and; Fc_{b} comprises a CH2_{b} and a CH3_{b} immunoglobulin heavy chain constant domain wherein the CH2_{b} domain is a second CH2 immunoglobulin heavy chain constant domain and the CH3_{b} domain is a second CH3 immunoglobulin heavy chain constant domain.

In some aspects, the heavy chain variable domain that specifically binds a T cell co-stimulatory molecule is an immunogloblulin heavy chain variable domain. In some aspects, the heavy chain variable domain that specifically binds a T cell co-stimulatory molecule is a single domain sequence. In particular aspects, the heavy chain variable domain that specifically binds a T cell co-stimulatory molecule is a nanobody. In particular aspects, the heavy chain variable domain that specifically binds a T cell co-stimulatory molecule is a camelid. In particular aspects, the heavy chain variable domain that specifically binds a T cell co-stimulatory molecule is a single-domain variable new antigen receptor. In various non-limiting embodiments, V_{HHb} comprises a heavy chain CDR1 (HCDR1), a heavy chain CDR2 (HCDR2), a heavy chain CDR3 (HCDR3) comprising the amino acid sequence of SEQ ID NO: 24, SEQ ID NO: 25, and SEQ ID NO: 26 respectively; or SEQ ID NO: 105, SEQ ID NO: 106 and SEQ ID NO: 107, respectively. In various other non-limiting embodiments, V_{HHb} comprises an amino acid sequence at least 90%, at least 91% at least 92%, at least 93%, at least 94%, at least 95%, at least 96%, at least 97%, at least 98%, or at least 99% identical to SEQ ID NO: 52 or 103, 137, or 100% identical to SEQ ID NO:27 or 104.

In some aspects, the V_{HHb} is SEQ ID NO: 27 is further modified to further enhance manufacturability. In some aspects, the V_{HHb} is SEQ ID NO:27 but further comprises a D30R substitution. In some aspects, the V_{HHb} is SEQ ID NO: 27 but further comprises a D30P substitution. In some aspects, the V_{HHb} is SEQ ID NO: 27 but further comprises a S75A substitution. In some aspects, the V_{HHb} is SEQ ID NO: 27 but further comprises a Y102I substitution. In some aspects, the V_{HHb} is SEQ ID NO: 27 but further comprises a S100G substitution. In some aspects, the V_{HHb} is SEQ ID NO: 27 but further comprises a L₁₀₁A substitution. In some aspects, the V_{HHb} is SEQ ID NO: 27 but further comprises a Q106N substitution. In some aspects, the V_{HHb} is SEQ ID NO: 27 but further comprises a D31S substitution. In some aspects, the V_{HHb} is SEQ ID NO: 27 but further comprises a S100P substitution. In some aspects, the V_{HHb} is SEQ ID NO: 27 but further comprises a R52T substitution. In some aspects, the sequence comprises one or more substitutions selected from D30R, D30P, S75A, Y102I, S100G, L101A, Q106N, D31S, S100P, OR R52T. In some aspects, the V_{HHB} is selected from SEQ ID NOs: 50-58.

In some aspects, the heavy chain variable domain that specifically binds a T cell receptor binding site is an immunoglobulin heavy chain variable domain. In some aspects, the heavy chain variable domain that specifically binds a T cell receptor binding site is a single domain sequence. In particular aspects, the heavy chain variable domain that specifically binds a T cell receptor binding site is a nanobody. In particular aspects, the heavy chain variable domain that specifically binds a T cell receptor binding site is a camelid. In particular aspects, the heavy chain variable domain that specifically binds a T cell receptor binding site is a single-domain variable new antigen receptor. In various non-limiting embodiments, V_{HHa} comprises a heavy chain CDR1 (HCDR1), a heavy chain CDR2 (HCDR2), a heavy chain CDR3 (HCDR3) comprising the amino acid sequence of SEQ ID NO: 14, SEQ ID NO: 15, and SEQ ID NO: 16 respectively; SEQ ID NO: 37, SEQ ID NO: 15 and SEQ ID NO: 16 respectively; or SEQ ID NO: 109, SEQ ID NO: 110, and SEQ ID NO: 111 respectively, respectively. In various other non-limiting embodiments, V_{HHa} comprises the amino acid sequence at least 90%, at least 91% at least 92%, at least 93%, at least 94%, at least 95%, at least 96%, at least 97%, at least 98%, or at least 99% identical to any one of SEQ ID NOs: 17-23, and 108, or any one of SEQ ID NOs: 17-23, and 108.

In particular aspects provided herein are amino acid sequences with a conservative substitution in which there are up to 10, up to 8, up to 5, or up to 3 amino acids substituted by amino acids having analogical or similar properties, compared to the amino acid sequence of the sequences disclosed herein.

In some aspects, the light chain (V_{L}-C_{L}) associates with the V_{H} and C_{H1} of a heavy chain to form an “antigen binding arm” and the variable domains in the antigen binding arm interact to form the “antigen binding site”. As described herein, “antigen binding site” and “antigen binding domain” are used interchangeably.

### Lambda Charge Pairs

The terms “charge pair(s)” and “charge mutation(s)” are used interchangeably throughout this specification and refer to a positively charged amino acid residue and a negatively charged amino acid residue, one of which is located in the a light chain region (e.g. constant light chain region) and the other in a heavy chain region (e.g. constant heavy chain region 1 (C_{H1})) of an antigen binding arm, located at positions intended to promote association of the light and heavy chains. By “lambda charge pair”, it is meant a charge pair where a positively or negatively charged amino acid residue is located in a lambda light chain. By “kappa charge pair”, it is meant a charge pair where a positively or negatively charged amino acid residue in the light chain is located in a kappa light chain.

Without wishing to be bound by theory, it is believed that the oppositely charged amino acid residues in the charge pair increase the attraction of the heavy chain to the light chain in an antigen binding arm, thereby promoting formation of the antigen binding arm with the correct heavy and light chain.

At least one of the amino acid residues of the charge pair have been engineered into the antigen binding arm (i.e. at least one amino acid residue in the pair is not a wild-type amino acid residue). In some aspects, both amino acid residues in the charge pair are engineered into the antigen binding arm (i.e. both amino acid residues in the pair are not wild-type amino acid residues).

The amino acid residues of the charge pair are typically naturally occurring. Naturally occurring positively charged amino acid residues according to the present disclosure include arginine, lysine and histidine. Naturally occurring negatively charged amino acid residues according to the present disclosure include glutamic acid, serine, threonine and aspartic acid. Although serine and threonine are often described in the art as ‘uncharged’, they have an isoelectric point below 6 and therefore are partially negatively charged at neutral pH. For the purposes of the charge pairs disclosed herein, serine and threonine are examples of negatively charged amino acid residues (together with glutamic acid and aspartic acid).

Hence, a charge pair may comprise a positively charged amino acid residue selected from arginine, lysine or histidine located at one of the positions in the charge pair and a negatively charged amino acid residue selected from aspartic acid, glutamic acid, serine or threonine located at the other position in the charge pair. For example, the charge pair may comprise any one of the following pairs of amino acid residues:
arginine and aspartic acid; arginine and glutamic acid; arginine and serine; arginine and threonine; lysine and aspartic acid; lysine and glutamic acid; lysine and serine; lysine and threonine; histidine and aspartic acid; histidine and glutamic acid; histidine and serine; and histidine and threonine.

In some aspects, the positively charged amino acid residue in the charge pair is located on the light chain and the negatively charged amino acid residue in the charge pair is located on the heavy chain. In other aspects, the negatively charged amino acid residue is located on the light chain and the positively charged amino acid residue in the charge pair is located on the heavy chain.

The binding proteins described herein may comprise a lambda charge pair in one of the antigen binding arms. As exemplified herein, lambda charge pairs can be introduced at several positions to improve pairing of the correct light and heavy chains in the antigen binding arm.

In some aspects, the lambda charge pair comprises a positively or negatively charged amino acid residue at position 117, 119, 134, 136 or 178 of the constant light chain lambda region (CLλ). In some aspects, the lambda charge pair comprises a positively or negatively charged amino acid residue at position 141, 185, 128, 145, 183, 185, 173, or 187 of C_{H1a} or C_{H1b}. As noted elsewhere, the numbering is according to Eu numbering.

In some embodiments, the C_{L1a} of the first polypeptide chain and/or the C_{L1b} of the second polypeptide chain comprises a constant light chain lambda region (CLλ); and the binding protein comprises at least one lambda charge pair wherein the lambda charge pair is either between the CLλ of the first polypeptide chain and a corresponding C_{H1a} of the third polypeptide chain, or the lambda charge pair is between the CLλ of the second polypeptide and a corresponding C_{H1b} of the fourth polypeptide chain, and where the lambda charge pair is located at one or more of the following pairs of positions: (i) position 117 in the CLλ and position 141 in the corresponding C_{H1a} or C_{H1b}; (ii) position 117 in the CLλ and position 185 in the corresponding C_{H1a} or C_{H1b}; (iii) position 119 in the CLλ and position 128 in the corresponding C_{H1a} or C_{H1b}; (iv) position 134 in the CLλ and position 128 in the corresponding C_{H1a} or C_{H1b}; (v) position 134 in the CLλ and position 145 in the corresponding C_{H1a} or C_{H1b}; (vi) position 134 in the CLλ and position 183 in the corresponding C_{H1a} or C_{H1b}; (vii) position 136 in the CLλ and position 185 in the corresponding C_{H1a} or C_{H1b}; (viii) position 178 in the CLλ and position 173 in the corresponding C_{H1a} or C_{H1b}; and (ix) position 117 in the CLλ and position 187 in the corresponding C_{H1a} or C_{H1b}; wherein the lambda charge pair comprises a positively charged amino acid residue selected from arginine, lysine and histidine located at one of the positions in the lambda charge pair and a negatively charged amino acid residue selected from aspartic acid, glutamic acid, serine and threonine located at the other position in the lambda charge pair; and wherein the numbering is according to the Eu index. In some embodiments, the lambda charge pair is selected from the following list (a) arginine at position 117 of the CLλ and aspartic acid at position 141 in the corresponding C_{H1a} or C_{H1b}; (b) arginine at position 117 of the CLλ and glutamic acid at position 141 in the corresponding C_{H1a} or C_{H1b}; (c) arginine at position 117 of the CLλ and serine at position 141 in the corresponding C_{H1a} or C_{H1b}; (d) arginine at position 117 of the CLλ and threonine at position 141 in the corresponding C_{H1a} or C_{H1b}; (e) lysine at position 117 of the CLλ and aspartic acid at position 141 in the corresponding C_{H1a} or C_{H1b}; (f) lysine at position 117 of the CLλ and glutamic acid at position 141 in the corresponding C_{H1a} or C_{H1b}; (g) lysine at position 117 of the CLλ and serine at position 141 in the corresponding C_{H1a} or C_{H1b}; and (h) lysine at position 117 of the CLλ and threonine at position 141 in the corresponding C_{H1a} or C_{H1b}.

In some aspects, the CLλ of the first polypeptide chain and/or CLλ of the second polypeptide chain each comprises an amino acid sequence having at least 90%, 91%, 92%, 93%, 94%, 95%, 96%, 97%, 98%, or 99% identity to SEQ ID NOs: 64-65, or comprise a CLλ chain according to SEQ ID NO: 64 or SEQ ID NO: 65. In some embodiments, CLλ comprises an amino acid sequence according to SEQ ID NO: 71, and the corresponding C_{H1a} or C_{H1b} comprises an amino acid sequence according to SEQ ID NO: 70 or SEQ ID NO: 132.

In some embodiments, CLλ comprises an amino acid sequence according to SEQ ID NO: 73, and the corresponding C_{H1a} or C_{H1b} comprises an amino acid sequence according to SEQ ID NO: 72.

### Engineered Disulfides

In some aspects, the binding proteins contain engineered disulfides in addition to or instead of the lambda charge pairs. By “engineered disulfides” it is meant that a native inter-chain disulfide bond at the C_{H1}—C_{L} (C_{H1a}—C_{L1a} or C_{H1b}—C_{L1b}) interface (e.g. at 220 of the C_{H1a} or C_{H1b} and 212 of the C_{L1a} or C_{L1b}) of one of the antigen binding arms has been replaced by an engineered (non-native) interchain disulfide, while the other antigen binding arm contains the native interchain disulfide bond at the C_{H1}—C_{L} interface. An engineered disulfide is typically formed by engineering cysteines into the CL of a light chain and the C_{H1} of the corresponding heavy chain and replacing the cysteines that normally form the interchain disulfide. Disclosure related to the introduction of engineered disulfide into binding proteins for the purpose of promoting heterodimerization can found e.g., in U.S. Pat. No. 9,527,927 and Mazor, 2015, which are herein incorporated by reference in their entirety.

The formation of disulfide bonds between cysteine residues occurs during the folding of many proteins that enter the secretory pathway. As the polypeptide chain collapses, cysteines brought into proximity can form covalent linkages during a process catalyzed by members of the protein disulfide isomerase family. The term “disulfide link” or “disulfide linked” as used herein, refers to the single covalent bond formed from the coupling of thiol groups, especially of cysteine residues. In some aspects, the covalent linkage between two cysteines is between the two sulfur atoms of each residue. However, depending on the environment, not all protein species may have a disulfide present at all times, for example, in the event of disulfide reduction. Thus, the term “disulfide link” or “disulfide linked” (whether native or engineered), in some aspects, also refers to the presence of two cysteine residues that are capable of forming a disulfide link, irrespective of whether or not they are actually linked at that individual point in time.

In some aspects herein, “V12 DS” or “V12” as used herein refers to removal of the native interchain disulfide bond in the C_{H1}/CLλ interface and the subsequent introduction of the alternative disulfide bond comprising four mutations: F126C/C220V in the C_{H1} domain, and mutations S122C/C212V in the lambda constant domain.

Thus, in some aspects: (i) the C_{L1a} of the first polypeptide chain and the C_{H1a} of the third polypeptide chain, wherein the C_{L1a} of the first polypeptide chain is a CLλ and wherein the pair of engineered cysteines are located at position 122 of the CLλ of the first polypeptide chain and position 126 of the C_{H1a} of the third polypeptide chain, and wherein the CLλ of the first polypeptide chain comprises a non-cysteine residue at position 212 and the C_{H1a} of the third polypeptide chain comprises a non-cysteine residue at position 220, optionally wherein the non-cysteine residues are valines; or (ii) the C_{L1b} of the second polypeptide chain and the C_{H1b} of the fourth polypeptide chain, wherein the C_{L1a} of the first polypeptide chain is a CLλ and wherein the pair of engineered cysteines are located at position 122 of the CLλ of the second polypeptide chain and position 126 of the C_{H1b} of the fourth polypeptide chain, and wherein the CLλ of the second polypeptide chain comprises a non-cysteine residue at position 212 and the C_{H1b} of the fourth polypeptide chain comprises a non-cysteine residue at position 220, optionally wherein the non-cysteine residues are valines.

In the binding proteins antibodies exemplified herein, the engineered disulfide may be present on the “first” antigen binding arm containing the lambda charge pairs and the native disulfide is present on the “second” antigen binding arm that does not contain the lambda charge pairs. However, the opposite arrangement is also specifically contemplated, i.e. where the native disulfide is present on the first antigen binding arm and the engineered disulfide is present on the second antigen binding arm.

In some aspects, the pair of cysteines are engineered into a constant light chain kappa region (CLκ) and CH1 and are located at position 121 of the CLκ and position 126 of the CH1, and wherein the same CLκ comprises a non-cysteine residue at position 214 and the same CH1 comprises a non-cysteine residue at position 220. In some aspects, the non-cysteine residues are valines.

In some aspects, there is provided a binding protein, wherein either: (i) a disulfide link between the C_{L1a} of the first polypeptide chain and the C_{H1a} of the third polypeptide chain is formed between a pair of cysteines engineered into the C_{L1a} of the first polypeptide chain and the C_{H1a} of the third polypeptide chain, and a disulfide link between the C_{L2} of the second polypeptide chain and the C_{H1b} of the fourth polypeptide chain is formed between a pair of native cysteines; or (ii) a disulfide link between the C_{L1b} of the second polypeptide chain and the C_{H1b} of the fourth polypeptide chain is formed between a pair of cysteines engineered into the C_{L1b} of the second polypeptide chain and the C_{H1b} of the fourth polypeptide chain, and a disulfide link between the C_{L1a} of the first polypeptide chain and the C_{H1a} of the third polypeptide chain is formed between a pair of native cysteines.

In some aspects, the pair of cysteines are engineered into either: (i) the C_{L1a} of the first polypeptide chain and the C_{H1a} of the third polypeptide chain, wherein the C_{L1a} of the first polypeptide chain is a CLκ and wherein the pair of engineered cysteines are located at position 121 of the CLκ of the first polypeptide chain and position 126 of the C_{H1a} of the third polypeptide chain, and wherein the CLκ of the first polypeptide chain comprises a non-cysteine residue at position 214 and the C_{H1a} of the third polypeptide chain comprises a non-cysteine residue at position 220, optionally wherein the non-cysteine residues are valines; or (ii) the C_{L1b} of the second polypeptide chain and the C_{H1b} of the fourth polypeptide chain, wherein the C_{L1b} of the first polypeptide chain is a CLκ and wherein the pair of engineered cysteines are located at position 121 of the CLκ of the second polypeptide chain and position 126 of the C_{H1b} of the fourth polypeptide chain, and wherein the CLκ of the second polypeptide chain comprises a non-cysteine residue at position 214 and the C_{H1b} of the fourth polypeptide chain comprises a non-cysteine residue at position 220, optionally wherein the non-cysteine residues are valines; wherein the numbering is according to the Eu index.

### Kappa Charge Pairs

In some aspects, the antigen binding arm comprises a constant light chain kappa region (CLκ). As described herein, techniques such as light chain affinity chromatography that utilizes affinity resins specific for either CLκ or CLλ can be used to selectively purify antibodies based on their light chain. Examples of such affinity resins include the LambdaFabSelect and KappaSelect resins available from GE Healthcare. Such methods can be used to selectively purify binding proteins containing both CLκ and CLλ and can therefore be used to improve production of bispecific antibodies in this format.

In some embodiments, the C_{L1a} of the first polypeptide chain and/or the C_{L1b} of the second polypeptide chain comprises a constant light chain kappa region (CLκ); and a kappa charge pair wherein the kappa charge pair is either between the CLκ of the first polypeptide chain and the corresponding C_{H1a} of the third polypeptide chain, or the kappa charge pair is between the CLκ of the second polypeptide and the corresponding C_{H1b} of the fourth polypeptide chain; wherein the kappa charge pair is located at position 117 in the CLκ and position 141 in the corresponding C_{H1a} or C_{H1b} and comprises a positively charged amino acid residue selected from arginine, lysine, and histidine located at one of the positions in the kappa charge pair, and a negatively charged amino acid residue selected from aspartic acid, glutamic acid, serine and threonine located at the other position in the kappa charge pair.

In some embodiments, the negatively charged amino acid residue in the kappa charge pair is located at position 133 of the CLκ, and the positively charged amino acid residue is located at position 183 of the corresponding C_{H1a} or C_{H1b}, optionally wherein the negatively charged amino acid residue at position 133 of the CLκ is a glutamic acid, and optionally wherein the positively charged amino acid residue at position 183 of the corresponding C_{H1a} or C_{H1b} is a lysine.

In some embodiments, the CLκ of the first polypeptide chain or the second polypeptide chain comprises an amino acid sequence having at least 90%, 91%, 92%, 93%, 94%, 95%, 96%, 97%, 98%, or 99% identity to SEQ ID NO: 66, or comprise a CLκ chain according to SEQ ID NO: 66.

In some embodiments, the corresponding C_{H1a} or C_{H1b} in each of the one or more lambda charge pairs comprises an amino acid sequence having at least 90%, 91%, 92%, 93%, 94%, 95%, 96%, 97%, 98%, or 99% to SEQ ID NO: 64-65 or is SEQ ID NO: 64 or SEQ ID NO: 65, and/or the corresponding C_{H1a} or C_{H1b} in the kappa charge pair comprises an amino acid sequence having at least 90%, 91%, 92%, 93%, 94%, 95%, 96%, 97%, 98%, or 99% to SEQ ID NO: 66 or is SEQ ID NO: 66.

### Compositions and Methods of Treatment

The binding proteins of the disclosure may be prepared using domains or sequences obtained or derived from any human or non-human antibody, including, for example, human, murine, or humanized antibodies. Some aspects of the present disclosure relate to an isolated nucleic acid molecule encoding a binding protein as described herein. In various aspects the isolated nucleic acid molecules comprise a nucleotide sequence according to any one of SEQ ID NOs: 118-121. In some embodiments, the isolated nucleic acid molecules comprise the nucleotide sequence according to any one of SEQ ID NOs: 122-125.

In some aspects, the methods disclosed herein relate to treating a subject for cancer by administering an effective amount of a binding protein. The disclosure also provides a therapeutically effective amount of a binding protein for use in treating cancer in a subject. The disclosure also provides use of a binding protein and/or pharmaceutical composition for the manufacture of a medicament. The disclosure also provides use of a binding protein and/or pharmaceutical composition for the manufacture of a medicament for treating cancer. In some embodiments, the cancer is a B cell malignancy. In other embodiments, the cancer is multiple myeloma.

In some aspects, the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, primary mediastinal large B cell lymphoma (PMBCL) or small lymphocytic lymphoma (SLL). In one embodiment, the B-cell malignancy is non-Hodgkin's lymphoma (NHL). In another embodiment, the B-cell malignancy is chronic lymphocytic leukemia (CLL). In another embodiment, the B-cell malignancy is mantle cell lymphoma (MCL). In another embodiment, the B-cell malignancy is diffuse large B-cell lymphoma (DLBCL). In another embodiment, the B-cell malignancy is follicular lymphoma (FL). In another embodiment, the B-cell malignancy is Burkett lymphoma. In another embodiment, the B-cell malignancy is primary mediastinal large B cell lymphoma (PMBCL). In a further embodiment, the B-cell malignancy is small lymphocytic lymphoma (SLL).

In various non-limiting embodiments, the CLL, MCL, FL, and/or DLBCL is positive for AA amyloidosis. In various other non-limiting embodiments, the CLL, MCL, FL, and/or DLBCL is fibrin-associated (FA).

In some aspects, the methods disclosed herein relate to treating a subject for an inflammatory disease and/or autoimmune disorder where B cells are involved by administering an effective amount of a binding protein. The disclosure also provides a therapeutically effective amount of a binding protein for use in treating an inflammatory disease and/or autoimmune disorder where B cells are involved in a subject.

In some aspects, the methods disclosed herein relate to treating a subject for an inflammatory and/or autoimmune disorder by administering an effective amount of a binding protein. The disclosure also provides a therapeutically effective amount of a binding protein for use in treating an inflammatory and/or autoimmune disorder in a subject. The disclosure also provides use of a binding protein and/or pharmaceutical composition for the manufacture of a medicament for treating an inflammatory and/or autoimmune disorder.

In some aspects, the inflammatory disease and/or autoimmune disorder is scleroderma, systemic lupus erythematosus (SLE), myositis, rheumatoid arthritis (RA), Sjogren's syndrome or anti-neutrophil cytoplasmic autoantibody (ANCA) vasculitis. In one embodiment, the inflammatory disease and/or autoimmune disorder is scleroderma. In another embodiment, the inflammatory disease and/or autoimmune disorder is systemic lupus erythematosus (SLE). In another embodiment, the inflammatory disease and/or autoimmune disorder is myositis. In another embodiment, the inflammatory disease and/or autoimmune disorder is rheumatoid arthritis (RA). In another embodiment, the inflammatory disease and/or autoimmune disorder is Sjogren's syndrome. In a further embodiment, the inflammatory disease and/or autoimmune disorder anti-neutrophil cytoplasmic autoantibody (ANCA) vasculitis.

In the method of treatment of the present disclosure, the binding proteins can preferentially activate a subset of T cells in the subject. The subset of T cells can be CD8+ T cells. The CD8+ T cells can be preferentially activated as compared to CD4 T cells. The preferential activation of CD8+ T cells reduces engagement with tumor promoting T cells and CD4+ T cells which produce the majority of cytokines that cause release syndrome (CRS). Additionally, the preferential engagement of the CD8+ T cells induce pyroptosis.

The activation of T cells through methods of the present disclosure can be determined by measuring the percentage of surface interleukin-2 receptor alpha chain-positive (CD25+) T cells. The percentage of surface CD25+ T cells that are CD8 T cells can be higher than the percentage of surface CD25+ T cells that are CD4 T cells. In particular aspects, activation of T cells can be determined by the percentage of CD69+/CD25+ T cells. In particular aspects, activation of T cells can be determined by measuring levels of cytokines released by activated T cells.

The method of treatment of the present disclosure can result in reduced engagement of regulatory T cells (Tregs), increased cytolytic activity, and/or reduced incidence of cytokine release syndrome (CRS) relative to that resulting from bispecific T-cell engager (BiTEs) previously known in the art. The disclosure also provides a therapeutically effective amount of a binding protein for use in reducing engagement of regulatory T cells (Tregs), increasing cytolytic activity, and/or reducing incidence of cytokine release syndrome (CRS) relative to that resulting from bispecific T-cell engager (BiTEs) previously known in the art.

In some aspects, the first tumor-associated antigen is CD20 and the second tumor-associated antigen is B cell maturation antigen (BCMA); and the inflammatory and/or autoimmune disorder involves B cells.

In some aspects, the first tumor-associated antigen is CD20 and the second tumor-associated antigen is B cell maturation antigen (BCMA); and the autoimmune disorder is scleroderma, systemic lupus erythematosus (SLE), myositis, rheumatoid arthritis (RA), Sjogren's syndrome or anti-neutrophil cytoplasmic autoantibody (ANCA) vasculitis.

In various aspects the binding proteins, compositions, pharmaceutical compositions, and/or medicaments can be administered using any suitable route of administration, including but not limited to oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants. In one aspect, the binding protein is administered via subcutaneous administration. In another aspect, the binding protein is administered via intravenous administration.

In view of the present disclosure, the binding proteins, pharmaceutical compositions, nucleic acid molecules, vectors, methods, compositions for use, and medicaments described herein can be configured by the person of ordinary skill in the art to meet the desired need.

In view of the above, the present invention also relates to the following clauses:

Clause 1. A binding protein comprising four polypeptide chains that form two different tumor-associated antigen (TAA) binding sites, a T cell receptor binding site, and a T cell co-stimulatory molecule binding site; wherein one tumor-associated antigen (TAA) binding site binds to B-cell maturation antigen (BCMA) and the other tumor-associated antigen (TAA) binding site binds to CD20.

Clause 2. The binding protein of clause 1, wherein the first polypeptide chain has a structure represented by the formula:

V_{L1}—C_{L1a}
a second polypeptide chain has a structure represented by the formula:

V_{L2}—C_{L1b}
a third polypeptide chain has a structure represented by the formula:

V_{H1}—C_{H1a}—V_{HHa}—Fcₐ
and a fourth polypeptide chain which has a structure represented by the formula:

V_{H2}—C_{H1b}—V_{HHb}—Fc_{b}
wherein the V_{L1} domain of the first polypeptide and the V_{H1} domain of the third polypeptide form the first of the two TAA binding sites, and the V_{L2} domain of the second polypeptide and the V_{H2} domain of the fourth polypeptide form the second of the two TAA binding sites, and
wherein:
V_{L1} is an immunoglobulin light chain variable domain and V_{H1} is an immunoglobulin heavy chain variable domain that together form a TAA binding domain that specifically binds to BCMA;
V_{L2} is an immunoglobulin light chain variable domain and V_{H2} is an immunoglobulin heavy chain variable domain that together form a TAA binding domain that specifically binds to CD20;
C_{L1a} and C_{L1b} are each an immunoglobulin light chain constant domain;
C_{H1a} and C_{H1L} are each an immunoglobulin CH1 heavy chain constant domain;
V_{HHa} is a single chain variable domain that specifically binds a T cell receptor (TCR);
V_{HHb} is a single chain variable domain that specifically binds a T cell co-stimulatory molecule;
Fcₐ comprises a C_{H2a} and a C_{H3a} immunoglobulin heavy chain constant domain, wherein the C_{H2a} domain is a first CH2 immunoglobulin heavy chain constant domain and the C_{H3a} domain is a first CH3 immunoglobulin heavy chain constant domain; and;
Fc_{b} comprises a C_{H2b} and a C_{H3b} immunoglobulin heavy chain constant domain wherein the C_{H2b} domain is a second CH2 immunoglobulin heavy chain constant domain and the C_{H3b} domain is a second CH3 immunoglobulin heavy chain constant domain.

Clause 3. The binding protein of clause 1, wherein the first polypeptide chain has a structure represented by the formula:

V_{L1}—C_{L1a}
a second polypeptide chain has a structure represented by the formula:

V_{L2}—C_{L1b}
a third polypeptide chain has a structure represented by the formula:

V_{H2}—C_{H1a}—V_{HHa}—Fcₐ
and a fourth polypeptide chain which has a structure represented by the formula:

V_{H1}—C_{H1b}—V_{HHb}—Fc_{b}
wherein the V_{L1} domain of the first polypeptide and the V_{H1} domain of the fourth polypeptide form the first of the two TAA binding sites, and the V_{L2} domain of the second polypeptide and V_{H2} domain of the third polypeptide form the second of the two TAA binding sites, and
wherein:
V_{L1} is an immunoglobulin light chain variable domain and V_{H1} is an immunoglobulin heavy chain variable domain that together form a TAA binding domain that specifically binds to BCMA;
V_{L2} is an immunoglobulin light chain variable domain and V_{H2} is an immunoglobulin heavy chain variable domain that together form a TAA binding domain that specifically binds to CD20;
C_{L1a} and C_{L1b} are each an immunoglobulin light chain constant domain;
C_{H1a} and C_{H1L} are each an immunoglobulin CH1 heavy chain constant domain;
V_{HHa} is a single chain variable domain that specifically binds a T cell receptor (TCR);
V_{HHb} is a single chain variable domain that specifically binds a T cell co-stimulatory molecule;
Fcₐ comprises a C_{H2a} and a C_{H3a} immunoglobulin heavy chain constant domain, wherein the C_{H2a} domain is a first CH2 immunoglobulin heavy chain constant domain and the C_{H3a} domain is a first CH3 immunoglobulin heavy chain constant domain; and;
Fc_{b} comprises a C_{H2b} and a C_{H3b} immunoglobulin heavy chain constant domain wherein the C_{H2b} domain is a second CH2 immunoglobulin heavy chain constant domain and the C_{H3b} domain is a second CH3 immunoglobulin heavy chain constant domain.

Clause 4. The binding protein of clause 1, wherein the first polypeptide chain has a structure represented by the formula:

V_{L2}—C_{L1a}
a second polypeptide chain has a structure represented by the formula:

V_{L1}—C_{L1b}
a third polypeptide chain has a structure represented by the formula:

V_{H2}—C_{H1a}—V_{HHa}—Fcₐ
and a fourth polypeptide chain which has a structure represented by the formula:

V_{H1}—C_{H1b}—V_{HHb}—Fc_{b}
wherein the V_{L1} domain of the first polypeptide and the V_{H1} domain of the fourth polypeptide form the first of the two TAA binding sites, and the V_{L2} domain of the second polypeptide and V_{H2} domain of the third polypeptide form the second of the two TAA binding sites, and
wherein:
V_{L1} is an immunoglobulin light chain variable domain and V_{H1} is an immunoglobulin heavy chain variable domain that together form a TAA binding domain that specifically binds to BCMA;
V_{L2} is an immunoglobulin light chain variable domain and V_{H2} is an immunoglobulin heavy chain variable domain that together form a TAA binding domain that specifically binds to CD20;
C_{L1a} and C_{L1b} are each an immunoglobulin light chain constant domain;
C_{H1a} and C_{H1L} are each an immunoglobulin CH1 heavy chain constant domain;
V_{HHa} is a single chain variable domain that specifically binds a T cell receptor (TCR);
V_{HHb} is a single chain variable domain that specifically binds a T cell co-stimulatory molecule;
Fcₐ comprises a C_{H2a} and a C_{H3a} immunoglobulin heavy chain constant domain, wherein the C_{H2a} domain is a first CH2 immunoglobulin heavy chain constant domain and the C_{H3a} domain is a first CH3 immunoglobulin heavy chain constant domain; and;
Fc_{b} comprises a C_{H2b} and a C_{H3b} immunoglobulin heavy chain constant domain wherein the C_{H2b} domain is a second CH2 immunoglobulin heavy chain constant domain and the C_{H3b} domain is a second CH3 immunoglobulin heavy chain constant domain.

Clause 5. The binding protein of any one of clause 2-4, further comprising L₁, a linker positioned between C_{H1a} and V_{HHa} on the third polypeptide chain, and/or L₂, a linker positioned between V_{HHa} and Fcₐ on the third polypeptide chain, wherein L₁ and L₂ are each independently a linker.

Clause 6. The binding protein of any one of clause 2-5, further comprising L₃, a linker positioned between C_{H1b} and V_{HHb} on the fourth polypeptide chain, and/or L₄, a linker positioned between V_{HHb} and Fc_{b} on the fourth polypeptide chain, wherein L₃ and L₄ are each independently a linker.

Clause 7. The binding protein of clause 5 or clause 6, wherein L₁, L₂, L₃, and/or L₄ each independently comprise one or more repeats of the amino acid sequence of SEQ ID NO: 12 and/or SEQ ID NO: 13.

Clause 8. The binding protein of any one of clauses 2-7 further comprising H₁, an immunoglobulin hinge region positioned between C_{H1a} and V_{HHa} on the third polypeptide chain, and/or H₂, an immunoglobulin hinge region positioned between V_{HHa} and Fcₐ on the third polypeptide chain, wherein H₁ and H₂ are each independently an immunoglobulin hinge region.

Clause 9. The binding protein of clause 8, wherein H₁ comprises SEQ ID NO: 28, SEQ ID NO: 29, SEQ ID NO: 30, SEQ ID NO: 59, SEQ ID NO: 60 or SEQ ID NO: 116, and wherein H₂ comprises SEQ ID NO: 28, SEQ ID NO: 29, SEQ ID NO: 30, SEQ ID NO: 59 or SEQ ID NO: 60.

Clause 10. The binding protein of any one of clauses 2-9, further comprising H₃, an immunoglobulin hinge region positioned between C_{H1b} and V_{HHb} on the fourth polypeptide chain, and/or H₄, an immunoglobulin hinge region positioned between V_{HHb} and the Fc_{b} on the fourth polypeptide chain, wherein H₃ and H₄ are each independently an immunoglobulin hinge region.

Clause 11. The binding protein of clause 10, wherein H₃ comprises SEQ ID NO: 28, SEQ ID NO: 29, SEQ ID NO: 30, SEQ ID NO: 59, SEQ ID NO: 60 or SEQ ID NO: 116, and wherein H₄ comprises SEQ ID NO: 28, SEQ ID NO: 29, SEQ ID NO: 30, SEQ ID NO: 59 or SEQ ID NO: 60.

Clause 12. The binding protein of any one of clauses 1-2 or 5-11, wherein the first polypeptide chain has a structure represented by the formula

V_{L1}—C_{L1a}
a second polypeptide chain has a structure polypeptide chain represented by the formula:

V_{L2}—C_{L1b}
a third polypeptide chain has a structure represented by the formula:

V_{H1}—C_{H1a}—H₁-L₁-V_{HHa}—H₂-L₂-Fcₐ or

V_{H1}—C_{H1a}—H₁-L₁-V_{HHa}-L₂-H₂-Fcₐ
and a fourth polypeptide chain has a structure represented by the formula:

V_{H2}—C_{H1b}—H₃-L₃-V_{HHb}—H₄-L₄-Fc_{b} or

V_{H2}—C_{H1b}—H₃-L₃-V_{HHb}-L₄-H₄—Fc_{b}.

Clause 13. The binding protein of any one of clauses 1 or 4-11, wherein the first polypeptide chain has a structure represented by the formula

V_{L2}—C_{L1a}
a second polypeptide chain has a structure polypeptide chain represented by the formula:

V_{L1}—C_{L1b}
a third polypeptide chain has a structure represented by the formula:

V_{H2}—C_{H1a}—H₁-L₁-V_{HHa}—H₂-L₂-Fcₐ or

V_{H2}—C_{H1a}—H₁-L₁-V_{HHa}-L₂-H₂-Fcₐ
and a fourth polypeptide chain has a structure represented by the formula:

V_{H1}—C_{H1b}—H₃-L₃-V_{HHb}—H₄-L₄-Fc_{b} or

V_{H1}—C_{H1b}—H₃-L₃-V_{HHb}-L₄-H₄—Fc_{b}.

Clause 14. The binding protein of any one of clauses 2-11, wherein the Fcₐ and/or Fc_{b} is an IgG isotype.

Clause 15. The binding protein of clause 14, wherein the Fcₐ and/or the Fc_{b} is an IgG1 isotype.

Clause 16. The binding protein of any one of clauses 2-15, wherein the binding protein activates T cells only when bound to a tumor associated antigen at one or both of the tumor-associated antigen binding sites.

Clause 17. The binding protein of any one of clauses 2-16, wherein the V_{HHa} comprises a heavy chain CDR1 (HCDR1), a heavy chain CDR2 (HCDR2) and a heavy chain CDR3 (HCDR3), comprising the amino acid sequences of SEQ ID NO: 14, SEQ ID NO: 15 and SEQ ID NO: 16, respectively.

Clause 18. The binding protein of any one of clauses 2-16, wherein the V_{HHa} comprises a heavy chain CDR1 (HCDR1), a heavy chain CDR2 (HCDR2) and a heavy chain CDR3 (HCDR3) comprising the amino acid sequences of SEQ ID NO: 37, SEQ ID NO: 15 and SEQ ID NO: 16, respectively.

Clause 19. The binding protein of any one of clauses 2-18, wherein the V_{HHa} comprises the amino acid sequence at least 90%, at least 91% at least 92%, at least 93%, at least 94%, at least 95%, at least 96%, at least 97%, at least 98%, or at least 99% identical to any one of SEQ ID NOs: 17-23, or any one of SEQ ID NOs: 17-23.

Clause 20. The binding protein of any one of clauses 2-16, wherein the V_{HHa} comprises a heavy chain CDR1 (HCDR1), a heavy chain CDR2 (HCDR2) and a heavy chain CDR3 (HCDR3), comprising the amino acid sequences of SEQ ID NO: 109, SEQ ID NO: 110, and SEQ ID NO: 111 respectively.

Clause 21. The binding protein of any one of clauses 2-16 or 20, wherein the V_{HHa} comprises the amino acid sequence at least 90%, at least 91% at least 92%, at least 93%, at least 94%, at least 95%, at least 96%, at least 97%, at least 98%, or at least 99% identical to SEQ ID NO: 108, or SEQ ID NO: 108.

Clause 22. The binding protein of any one of clauses 2-21, wherein the T cell costimulatory molecule is CD8.

Clause 23. The binding protein of any one of clauses 2-22, wherein the V_{HHb} comprises a heavy chain CDR1 (HCDR1), a heavy chain CDR2 (HCDR2) and a heavy chain CDR3 (HCDR3) comprising the amino acid sequences of SEQ ID NO: 24, SEQ ID NO: 25 and SEQ ID NO: 26, respectively.

Clause 24. The binding protein of any one of clauses 2-22, wherein the V_{HHb} comprises a heavy chain CDR1 (HCDR1), a heavy chain CDR2 (HCDR2) and a heavy chain CDR3 (HCDR3) comprising the amino acid sequences of SEQ ID NO: 105, SEQ ID NO: 106 and SEQ ID NO: 107, respectively.

Clause 25. The binding protein of any one of clauses 2-24, wherein the V_{HHb} comprises an amino acid sequence at least 90%, at least 91% at least 92%, at least 93%, at least 94%, at least 95%, at least 96%, at least 97%, at least 98%, or at least 99% identical to SEQ ID NO: 104, or SEQ ID NO: 104.

Clause 26. The binding protein of any one of clauses 2-25, wherein C_{H3a} and C_{H3b} in each of Fcₐ and Fc_{b} comprise modifications to facilitate heterodimerization of Fcₐ and Fc_{b}.

Clause 27. The binding protein of clause 26, wherein the modifications are substitutions to generate a knob in one of Fcₐ and Fc_{b} and a hole in the other of Fcₐ and Fc_{b}, wherein the knob is a substitution to tryptophan at position 366, and wherein the hole is a substitution of one or more of the following:
(i) a substitution to valine at position 407;
(ii) a substitution to serine at position 366; and
(iii) a substitution to alanine at position 368
and wherein the numbering is according to the Eu index.

Clause 28. The binding protein of clause 27, wherein the Fcₐ or the Fc_{b} containing the knob further comprises a cysteine at position 354 and/or the Fcₐ or the Fc_{b} containing the hole comprises a cysteine at position 349, and wherein the numbering is according to the Eu index.

Clause 29. The binding protein of any one of clauses 2-28 wherein the C_{H2a} and C_{H2b} immunoglobulin heavy chain constant domains each comprise the following substitutions: E233P/L234V/L235A/G236del/S267K, and wherein the numbering is according to the Eu index.

Clause 30. The binding protein of any one of clauses 2-29, wherein either the C_{H3a} immunoglobulin heavy chain constant domain or C_{H3b} immunoglobulin heavy chain constant domain comprises a H435R and Y436F substitution, and wherein the numbering is according to the Eu index.

Clause 31. The binding protein of any one of clauses 2-30, wherein Fcₐ comprises the amino acid sequence of SEQ ID NO: 31 or SEQ ID NO: 32.

Clause 32. The binding protein of any one of clauses 2-31, wherein Fc_{b} comprises the amino acid sequence of SEQ ID NO: 33.

Clause 33. The binding protein of any one of clauses 2-32, wherein the third polypeptide chain comprises either SEQ ID NO: 34 or SEQ ID NO: 35 and the fourth polypeptide chain comprises SEQ ID NO: 36.

Clause 34. The binding protein of any one of clauses 2-33, wherein the C_{L1a} of the first polypeptide chain and/or the C_{L1b} of the second polypeptide chain comprises a constant light chain lambda region (CLλ); and the binding protein comprises at least one lambda charge pair wherein the lambda charge pair is either between the CLλ of the first polypeptide chain and a corresponding C_{H1a} of the third polypeptide chain, or the lambda charge pair is between the CLλ of the second polypeptide and a corresponding C_{H1b} of the fourth polypeptide chain, and where the lambda charge pair is located at one or more of the following pairs of positions:
(i) position 117 in the CLλ and position 141 in the corresponding C_{H1a} or C_{H1b};
(ii) position 117 in the CLλ and position 185 in the corresponding C_{H1a} or C_{H1b}; (ii)
(iii) position 119 in the CLλ and position 128 in the corresponding C_{H1a} or C_{H1b};
(iv) position 134 in the CLλ and position 128 in the corresponding C_{H1a} or C_{H1b};
(v) position 134 in the CLλ and position 145 in the corresponding C_{H1a} or C_{H1b};
(vi) position 134 in the CLλ and position 183 in the corresponding C_{H1a} or C_{H1b};
(vii) position 136 in the CLλ and position 185 in the corresponding C_{H1a} or C_{H1b};
(viii) position 178 in the CLλ and position 173 in the corresponding C_{H1a} or C_{H1b}; and
(ix) position 117 in the CLλ and position 187 in the corresponding C_{H1a} or C_{H1b};
wherein the lambda charge pair comprises a positively charged amino acid residue selected from arginine, lysine and histidine located at one of the positions in the lambda charge pair and a negatively charged amino acid residue selected from aspartic acid, glutamic acid, serine and threonine located at the other position in the lambda charge pair; and wherein the numbering is according to the Eu index.

Clause 35. The binding protein of clause 34, wherein the lambda charge pair is selected from the following list:
(a) arginine at position 117 of the CLλ and aspartic acid at position 141 in the corresponding C_{H1a} or C_{H1b};
(b) arginine at position 117 of the CLλ and glutamic acid at position 141 in the corresponding C_{H1a} or C_{H1b};
(c) arginine at position 117 of the CLλ and serine at position 141 in the corresponding C_{H1a} or C_{H1b};
(d) arginine at position 117 of the CLλ and threonine at position 141 in the corresponding C_{H1a} or C_{H1b};
(e) lysine at position 117 of the CLλ and aspartic acid at position 141 in the corresponding C_{H1a} or C_{H1b};
(f) lysine at position 117 of the CLλ and glutamic acid at position 141 in the corresponding C_{H1a} or C_{H1b};
(g) lysine at position 117 of the CLλ and serine at position 141 in the corresponding C_{H1a} or C_{H1b}; and
(h) lysine at position 117 of the CLλ and threonine at position 141 in the corresponding C_{H1a} or C_{H1b}.

Clause 36. The binding protein of either clause 34 or clause 35, wherein the CLλ of the first polypeptide chain and/or CLλ of the second polypeptide chain each comprises an amino acid sequence having at least 90%, 91%, 92%, 93%, 94%, 95%, 96%, 97%, 98%, or 99% identity to SEQ ID NOs: 64-65, or comprise a CLλ chain according to SEQ ID NO: 64 or SEQ ID NO: 65.

Clause 37. The binding protein of any one of clauses 34-36, wherein the CLλ comprises an amino acid sequence according to SEQ ID NO: 71, and the corresponding C_{H1a} or C_{H1b} comprises an amino acid sequence according to SEQ ID NO: 70 or SEQ ID NO: 132.

Clause 38. The binding protein of any one of clauses 2-37, wherein the C_{L1a} of the first polypeptide chain and/or the C_{L1b} of the second polypeptide chain comprises a constant light chain kappa region (CLκ); and a kappa charge pair wherein the kappa charge pair is either between the CLκ of the first polypeptide chain and the corresponding C_{H1a} of the third polypeptide chain, or the kappa charge pair is between the CLκ of the second polypeptide and the corresponding C_{H1b} of the fourth polypeptide chain; wherein the kappa charge pair is located at position 117 in the CLκ and position 141 in the corresponding C_{H1a} or C_{H1b} and comprises a positively charged amino acid residue selected from arginine, lysine, and histidine located at one of the positions in the kappa charge pair, and a negatively charged amino acid residue selected from aspartic acid, glutamic acid, serine and threonine located at the other position in the kappa charge pair.

Clause 39. The binding protein of clause 38, wherein the negatively charged amino acid residue in the kappa charge pair is located at position 133 of the CLκ, and the positively charged amino acid residue is located at position 183 of the corresponding C_{H1a} or C_{H1b}, optionally wherein the negatively charged amino acid residue at position 133 of the CLκ is a glutamic acid, and optionally wherein the positively charged amino acid residue at position 183 of the corresponding C_{H1a} or C_{H1b} is a lysine.

Clause 40. The binding protein of either clause 38 or clause 39, wherein the CLκ of the first polypeptide chain or the second polypeptide chain comprises an amino acid sequence having at least 90%, 91%, 92%, 93%, 94%, 95%, 96%, 97%, 98%, or 99% identity to SEQ ID NO: 66, or comprise a CLκ chain according to SEQ ID NO: 66.

Clause 41. The binding protein of any one of clauses 37-40 wherein the corresponding C_{H1a} or C_{H1b} in each of the one or more lambda charge pairs comprises an amino acid sequence having at least 90%, 91%, 92%, 93%, 94%, 95%, 96%, 97%, 98%, or 99% to SEQ ID NO: 64-65 or is SEQ ID NO: 64 or SEQ ID NO: 65, and/or the corresponding C_{H1a} or C_{H1b} in the kappa charge pair comprises an amino acid sequence having at least 90%, 91%, 92%, 93%, 94%, 95%, 96%, 97%, 98%, or 99% to SEQ ID NO: 66 or is SEQ ID NO: 66.

Clause 42. The binding protein of any one of clauses 2-41, wherein either:
(i) a disulfide link between the C_{L1a} of the first polypeptide chain and the C_{H1a} of the third polypeptide chain is formed between a pair of cysteines engineered into the C_{L1a} of the first polypeptide chain and the C_{H1a} of the third polypeptide chain, and a disulfide link between the C_{L2} of the second polypeptide chain and the C_{H1b} of the fourth polypeptide chain is formed between a pair of native cysteines; or
(ii) a disulfide link between the C_{L1b} of the second polypeptide chain and the C_{H1b} of the fourth polypeptide chain is formed between a pair of cysteines engineered into the C_{L1b} of the second polypeptide chain and the C_{H1b} of the fourth polypeptide chain, and a disulfide link between the C_{L1a} of the first polypeptide chain and the C_{H1a} of the third polypeptide chain is formed between a pair of native cysteines.

Clause 43. The binding protein of clause 42 wherein the pair of cysteines are engineered into either:
(i) the C_{L1a} of the first polypeptide chain and the C_{H1a} of the third polypeptide chain, wherein the C_{L1a} of the first polypeptide chain is a CLλ and wherein the pair of engineered cysteines are located at position 122 of the CLλ of the first polypeptide chain and position 126 of the C_{H1a} of the third polypeptide chain, and wherein the CLλ of the first polypeptide chain comprises a non-cysteine residue at position 212 and the C_{H1a} of the third polypeptide chain comprises a non-cysteine residue at position 220, optionally wherein the non-cysteine residues are valines; or
(ii) the C_{L1b} of the second polypeptide chain and the C_{H1b} of the fourth polypeptide chain, wherein the C_{L1b} of the second polypeptide chain is a CLλ and wherein the pair of engineered cysteines are located at position 122 of the CLλ of the second polypeptide chain and position 126 of the C_{H1b} of the fourth polypeptide chain, and wherein the CLλ of the second polypeptide chain comprises a non-cysteine residue at position 212 and the C_{H1b} of the fourth polypeptide chain comprises a non-cysteine residue at position 220, optionally wherein the non-cysteine residues are valines; wherein the numbering is according to the Eu index.

Clause 44. The binding protein of clause 42, wherein the pair of cysteines are engineered into either:
(i) the C_{L1a} of the first polypeptide chain and the C_{H1a} of the third polypeptide chain, wherein the C_{L1a} of the first polypeptide chain is a CLκ and wherein the pair of engineered cysteines are located at position 121 of the CLκ of the first polypeptide chain and position 126 of the C_{H1a} of the third polypeptide chain, and wherein the CLκ of the first polypeptide chain comprises a non-cysteine residue at position 214 and the C_{H1a} of the third polypeptide chain comprises a non-cysteine residue at position 220, optionally wherein the non-cysteine residues are valines; or
(ii) the C_{L1b} of the second polypeptide chain and the C_{H1b} of the fourth polypeptide chain, wherein the C_{L1b} of the first polypeptide chain is a CLκ and wherein the pair of engineered cysteines are located at position 121 of the CLκ of the second polypeptide chain and position 126 of the C_{H1b} of the fourth polypeptide chain, and wherein the CLκ of the second polypeptide chain comprises a non-cysteine residue at position 214 and the C_{H1b} of the fourth polypeptide chain comprises a non-cysteine residue at position 220, optionally wherein the non-cysteine residues are valines; wherein the numbering is according to the Eu index.

Clause 45. The binding protein of any one of clauses 41-43, wherein the CLλ comprises an amino acid sequence according to SEQ ID NO: 73, and the corresponding C_{H1a} or C_{H1b} comprises an amino acid sequence according to SEQ ID NO: 72.

Clause 46. The binding protein of any one of clauses 2-45, wherein the TAA binding domain that specifically binds to CD20 comprises a heavy chain CDR1 (HCDR1), a heavy chain CDR2 (HCDR2), a heavy chain CDR3 (HCDR3), a light chain CDR1 (LCDR1), a light chain CDR2 (LCDR2) and a light chain CDR3 (LCDR3), comprising the amino acid sequences of SEQ ID NO: 1, SEQ ID NO: 2, SEQ ID NO: 3, SEQ ID NO: 4, SEQ ID NO: 5 and SEQ ID NO: 6, respectively.

Clause 47. The binding protein of clause 46, wherein the TAA binding domain comprises a V_{H2} that is at least 90%, at least 91% at least 92%, at least 93%, at least 94%, at least 95%, at least 96%, at least 97%, at least 98%, or at least 99% identical to SEQ ID NO: 7; and a V_{L2} domain that is at least 90%, at least 91% at least 92%, at least 93%, at least 94%, at least 95%, at least 96%, at least 97%, at least 98%, or at least 99% identical to SEQ ID NO: 8; or comprises a V_{H2} domain according to SEQ ID NO: 7 and a V_{L2} domain according to SEQ ID NO: 8.

Clause 48. The binding protein of any one of clauses 2-47, wherein the TAA binding domain that specially binds to BCMA comprises a heavy chain CDR1 (HCDR1), a heavy chain CDR2 (HCDR2), a heavy chain CDR3 (HCDR3), a light chain CDR1 (LCDR1), a light chain CDR2 (LCDR2) and a light chain CDR3 (LCDR3), comprising the amino acid sequences of SEQ ID NO: 76, SEQ ID NO: 77, SEQ ID NO: 78, SEQ ID NO: 79, SEQ ID NO: 80 and SEQ ID NO: 81, respectively.

Clause 49. The binding protein of clause 48, wherein the TAA binding domain comprises a V_{H1} that is at least 90%, at least 91% at least 92%, at least 93%, at least 94%, at least 95%, at least 96%, at least 97%, at least 98%, or at least 99% identical to SEQ ID NO: 82; and a V_{L1} domain that is at least 90%, at least 91% at least 92%, at least 93%, at least 94%, at least 95%, at least 96%, at least 97%, at least 98%, or at least 99% identical to SEQ ID NO: 83; or comprises a V_{H1} domain according to SEQ ID NO: 82 and a V_{L1} domain according to SEQ ID NO: 83.

Clause 50. The binding protein of any one of clauses 2 or 5-49 comprising the amino acid sequences of SEQ ID NO: 10, SEQ ID NO: 84, SEQ ID NO: 11 and SEQ ID NO: 86.

Clause 51. The binding protein of clause 50, wherein the first polypeptide chain comprises the amino acid sequence of SEQ ID: 84, the second polypeptide chain comprises the amino acid sequence of SEQ ID: 10, the third polypeptide chain comprises the amino acid sequence of SEQ ID: 86, and the fourth polypeptide chain comprises the amino acid sequence of SEQ ID: 11.

Clause 52. The binding protein of any one of clauses 4-51 comprising the amino acid sequences of SEQ ID NO: 89, SEQ ID NO: 88, SEQ ID NO: 90 and SEQ ID NO: 91.

Clause 53. The binding protein of clause 52, wherein the first polypeptide chain comprises the amino acid sequence of SEQ ID: 89, the second polypeptide chain comprises the amino acid sequence of SEQ ID: 88, the third polypeptide chain comprises the amino acid sequence of SEQ ID: 91, and the fourth polypeptide chain comprises the amino acid sequence of SEQ ID: 90.

Clause 54. A pharmaceutical composition comprising the binding protein of any one of clauses 1-53 and a pharmaceutically acceptable carrier.

Clause 55. An isolated nucleic acid molecule encoding any one of the polypeptide chains of the binding protein of any one of clauses 1-53.

Clause 56. The isolated nucleic acid molecule according to clause 55 comprising the nucleotide sequence according to any one of SEQ ID NOs: 118-121.

Clause 57. The isolated nucleic acid molecule according to clause 55 comprising the nucleotide sequence according to any one of SEQ ID NOs: 122-125.

Clause 58. A vector comprising the isolated nucleic acid molecule of any one of clauses 56-57.

Clause 59. A method of treating cancer in a subject in need thereof, comprising administering a therapeutically effective amount of the binding protein of any one of clauses 1-53 or the pharmaceutical composition of clause 54 to the subject.

Clause 60. The method of clause 59, wherein the cancer is a B-cell malignancy.

Clause 61. The method of clause 60, wherein the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, primary mediastinal large B cell lymphoma (PMBCL) or small lymphocytic lymphoma (SLL).

Clause 62. The method of clause 59, wherein the cancer is multiple myeloma.

Clause 63. A method of treating an inflammatory disease and/or autoimmune disorder in a subject in need thereof, comprising administering a therapeutically effective amount of the binding protein of any one of clauses 1-53 or the pharmaceutical composition of clause 54 to the subject.

Clause 64. The method of clause 63, wherein the inflammatory disease and/or autoimmune disorder is scleroderma, systemic lupus erythematosus (SLE), myositis, rheumatoid arthritis (RA), Sjogren's syndrome or anti-neutrophil cytoplasmic autoantibody (ANCA) vasculitis.

Clause 65. The method of any one of clauses 59 to 64, wherein the binding protein preferentially activates a subset of T cells in the subject.

Clause 66. The method of clause 63, wherein the subset of T cells are CD8+ T cells.

Clause 67. The method of clause 66, wherein the CD8+ T cells are preferentially activated as compared to CD4+ T cells.

Clause 68. The method of any one of clauses 65-67, wherein the activation of T cells is determined by measuring the percentage of surface CD25+ T cells.

Clause 69. The method of clause 68, wherein the percentage of surface CD25+ T cells that are CD8+ T cells is higher than the percentage of surface CD25+ T cells that are CD4+ T cells.

Clause 70. The method of any one of clauses 59-69, wherein the binding protein is administered by subcutaneous administration.

Clause 71. The method of any one of clauses 59-69, wherein the binding protein is administered by intravenous administration.

Clause 72. A binding protein of any one of clauses 1-53, or the pharmaceutical composition of clause 54, for use as a medicament.

Clause 73. A binding protein of any one of clauses 1-53, or the pharmaceutical composition of clause 54, for use in the treatment of cancer.

Clause 74. The binding protein or pharmaceutical composition for use of clause 73, wherein the cancer is a B-cell malignancy.

Clause 75. The binding protein or pharmaceutical composition for use of clause 74, wherein the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, primary mediastinal large B cell lymphoma (PMBCL) or small lymphocytic lymphoma (SLL).

Clause 76. The binding protein or pharmaceutical composition for use of clause 73, wherein the cancer is multiple myeloma.

Clause 77. A binding protein of any one of clauses 1-53, or the pharmaceutical composition of clause 54, for use in the treatment of an inflammatory disease and/or autoimmune disorder.

Clause 78. The binding protein or pharmaceutical composition for use of clause 77, wherein the inflammatory disease and/or autoimmune disorder is scleroderma, systemic lupus erythematosus (SLE), myositis, rheumatoid arthritis (RA), Sjogren's syndrome or anti-neutrophil cytoplasmic autoantibody (ANCA) vasculitis.

Clause 79. Use of a binding protein of any one of clauses 1-53, or the pharmaceutical composition of clause 54, for the manufacture of a medicament for treating cancer.

Clause 80. The use of clause 79, wherein the cancer is a B-cell malignancy.

Clause 81. The use of clause 80, wherein the B-cell malignancy is non-Hodgkin's lymphoma (NHL), chronic lymphocytic leukemia (CLL), mantle cell lymphoma (MCL), diffuse large B-cell lymphoma (DLBCL), follicular lymphoma (FL), Burkett lymphoma, primary mediastinal large B cell lymphoma (PMBCL) or small lymphocytic lymphoma (SLL).

Clause 82. The use of clause 79, wherein the cancer is multiple myeloma.

Clause 83. Use of a binding protein of any one of clauses 1-53, or the pharmaceutical composition of clause 54, for the manufacture of a medicament for treating an inflammatory and/or autoimmune disorder.

Clause 84. The use of clause 83, wherein the inflammatory disease and/or autoimmune disorder is scleroderma, systemic lupus erythematosus (SLE), myositis, rheumatoid arthritis (RA), Sjogren's syndrome or anti-neutrophil cytoplasmic autoantibody (ANCA) vasculitis.

### EXAMPLES

The Examples that follow are illustrative of specific aspects of the disclosure, and various uses thereof. They are set forth for explanatory purposes only and should not be construed as limiting the scope of the disclosure in any way.

### Example 1: Binding Protein Structures and Sequences

Binding proteins of the disclosure are described in Table 1 below.

TABLE 1
Amino acid and Nucleic acid sequences for TPP-47648, TPP-47651, TPP-47646,
TPP47645

| SEQ | | |
|---|---|---|
| ID | | |
| NO: | Description | Sequence |
| 1 | TPP-47648 | SYNMH |
| | /TPP-47651/ | |
| | TPP-47645 | |
| | CD20 VH | |
| | CDR1 | |
| 2 | TPP-47648 | AIYPGSGDTSYNQKFKG |
| | /TPP-47651/ | |
| | TPP-47645 | |
| | CD20 VH | |
| | CDR2 | |
| 3 | TPP-47648 | VVYYSNSYWYFDV |
| | /TPP-47651/ | |
| | TPP-47645 | |
| | CD20 VH | |
| | CDR3 | |
| 4 | TPP- | RASSSVSYMH |
| | 47648/TPP- | |
| | 47651/TPP- | |
| | 47645 CD20 | |
| | VL CDR1 | |
| 5 | TPP-47648 | APSNLAS |
| | /TPP- | |
| | 47651/TPP- | |
| | 47645 CD20 | |
| | VL CDR2 | |
| 6 | TPP-47648 | QQWSFNPPT |
| | /TPP- | |
| | 47651/TPP- | |
| | 47645 CD20 | |
| | VL CDR3 | |
| 7 | TPP-47648 | EVQLVESGGGLVKPGGSLRLSCAASGFTFSSYSMNWVRQAP |
| | /TPP-47651 | GKGLEWVSSISGQSNYIYYADSVKGRFTISRDNAKNSLYLQM |
| | CD20 VH2 | NSLRAEDTAVYYCARGGNYFVEYFQYWGQGTLVTVSS |
| 8 | TPP-47648 | DIQMTQSPSSLSASVGDRVTITCRASSSVSYMHWYQQKPGK |
| | /TPP- | APKPLIYAPSNLASGVPSRFSGSGSGTDFTLTISSLQPEDFATY |
| | 47651/TPP- | YCQQWSFNPPTFGQGTKVEIK |
| | 47645 CD20 | |
| | VL2 | |
| 9 | TPP-47648 | EVQLVESGGGLVQPGGSLRLSCAASGYTFTSYNMHWVRQA |
| | /TPP-47651 | PGKGLEWVGAIYPGSGDTSYNQKFKGRFTISVDKSKNTLYL |
| | CD20 VH- | QMNSLRAEDTAVYYCARVVYYSNSYWYFDVWGQGTLVTV |
| | CH1 | SSASTKGPSVCPLAPSSKSTSGGTADLGCLVKDYFPEPVTVS |
| | | WNSGALTSGVHTFPAVLQSSGLYSLSSVVTVPSSSLGTQTYIC |
| | | NVNHKPSNTKVDKRV |
| 10 | TPP-47648 | DIQMTQSPSSLSASVGDRVTITCRASSSVSYMHWYQQKPGKA |
| | CD20 Light | PKPLIYAPSNLASGVPSRFSGSGSGTDFTLTISSLQPEDFATY |
| | Chain VL2- | YCQQWSFNPPTFGQGTKVEIKGQPKAAPSVRLFPPCSEELQA |
| | CL | NKATLVCLISDFYPGAVTVAWKADSSPVKAGVETTTPSKQS |
| | | NNKYAASSYLSLTPEQWKSHRSYSCQVTHEGSTVEKTVAPT |
| | | EVS |
| 11 | TPP-47648 | EVQLVESGGGLVQPGGSLRLSCAASGYTFTSYNMHWVRQA |
| | knob (CD8) | PGKGLEWVGAIYPGSGDTSYNQKFKGRFTISVDKSKNTLYL |
| | CD20 HC | QMNSLRAEDTAVYYCARVVYYSNSYWYFDVWGQGTLVTV |
| | CD8 (FC | SSASTKGPSVCPLAPSSKSTSGGTADLGCLVKDYFPEPVTVS |
| | knob) | WNSGALTSGVHTFPAVLQSSGLYSLSSVVTVPSSSLGTQTYIC |
| | | NVNHKPSNTKVDKRVEPKSVDKTGGSEVQLLESGGGLVQPG |
| | | GSLRLSCAASGFTFTDYAIGWFRQAPGKEREGVSCIRVSDGS |
| | | TYYADSVKGRFTISRDSSKNTLYLQMNSLRAEDTAVYYCAA |
| | | GSLYTCVQSIVWPARPYYDMDYWGQGTLVTVSSGGGGSTH |
| | | TCPPCPAPPVAGPSVFLFPPKPKDTLMISRTPEVTCVVVDVKH |
| | | EDPEVKFNWYVDGVEVHNAKTKPREEQYNSTYRVVSVLTV |
| | | LHQDWLNGKEYKCKVSNKALPAPIEKTISKAKGQPREPQVY |
| | | TLPPCREEMTKNQVSLWCLVKGFYPSDIAVEWESNGQPENN |
| | | YKTTPPVLDSDGSFFLYSKLTVDKSRWQQGNVFSCSVMHEA |
| | | LHNHYTQKSLSLSPGK |
| 12 | linker | TGGS |
| 13 | linker | GGGGS |
| 14 | TCR V_{HHa} | INFLG |
| | CDR1 | |
| 15 | TCR V_{HHa} | HISIGDQTDYADSAKG |
| | CDR2 | |
| 16 | TCR V_{HHa} | FSRIYPYDY |
| | CDR3 | |
| 17 | TCR V_{HHa} | EVQLVESGGGLVQPGGSLRLSCVASGDVHKINFLGWYRQAP |
| | | GKEREKVAHISIGDQTDYADSAKGRFTISRDESKNMVYLQM |
| | | NSLKPEDTAVYFCRAFSRIYPYDYWGQGTLVTVSS |
| 18 | TCR hu2.1 | EVQLLESGGGLVQPGGSLRLSCAASGDVHKINFLSWYRQAP |
| | V_{HHa} | GKEREKVAHISIGDQTDYADSAKGRFTISRDESKNTLYLQMN |
| | | SLRAEDTAVYYCRAFSRIYPYDYWGQGTLVTVSS |
| 19 | TCR hu2.2 | EVQLLESGGGLVQPGGSLRLSCAASGDVHKINFLGWYRQAP |
| | V_{HHa} | GKEREKVAHISIGDQTDYADSAKGRFTISRDESKNTLYLQMN |
| | | SLRAEDTAVYYCRAFSRIYPYDYWGQGTLVTVSS |
| 20 | TCR hu2.3 | EVQLLESGGGLVQPGGSLRLSCAASGDVHKINFLSWYRQAP |
| | V_{HHa} | GKGLEKVAHISIGDQTDYADSAKGRFTISRDESKNTLYLQMN |
| | | SLRAEDTAVYYCRAFSRIYPYDYWGQGTLVTVSS |
| 21 | TCR hu2.4 | EVQLLESGGGLVQPGGSLRLSCAASGDVHKINFLGWYRQAP |
| | V_{HHa} | GKGLEKVAHISIGDQTDYADSAKGRFTISRDESKNTLYLQMN |
| | | SLRAEDTAVYYCRAFSRIYPYDYWGQGTLVTVSS |
| 22 | TCR hu2.5 | EVQLLESGGGLVQPGGSLRLSCAASGDVHKINFLGWYRQAP |
| | V_{HHa} | GKEREKVAHISIGDQTDYADSAKGRFTISRDESKNTVYLQMN |
| | | SLRAEDTAVYFCRAFSRIYPYDYWGQGTLVTVSS |
| 23 | TCR hu2.6 | EVQLLESGGGLVQPGGSLRLSCAASGDVHKINFLGWYRQAP |
| | V_{HHa} | GKGLEKVAHISIGDQTDYADSAKGRFTISRDESKNTVYLQM |
| | | NSLRAEDTAVYFCRAFSRIYPYDYWGQGTLVTVSS |
| 24 | CD8 V_{HHb} | DYAIG |
| | CDR1 | |
| 25 | CD8 V_{HHb} | CIRVSDGSTYYADSVKG |
| | CDR2 | |
| 26 | CD8 V_{HHb} | GSLYTCVQSIVWPARPYYDMY |
| | CDR3 | |
| 27 | CD8 V_{HHb} | EVQLLESGGGLVQPGGSLRLSCAASGFTFDDYAIGWFRQAP |
| | | GKEREGVSCIRVSDGSTYYADSVKGRFTISRDNSKNTLYLQM |
| | | NSLRAEDTAVYYCAAGSLYTCVQSIVWPARPYYDMDYWGQ |
| | | GTLVTVSS |
| 28 | IgG1 hinge | DKTHTCPPCPAP |
| | sequence | |
| 29 | IgG1 Split | DK |
| | Hinge 1 | |
| 30 | IgG1 Split | THTCPPCPAP |
| | Hinge 2 | |
| 31 | Fcₐ (hole) | PVAGPSVFLFPPKPKDTLMISRTPEVTCVVVDVKHEDPEVKF |
| | | NWYVDGVEVHNAKTKPREEQYNSTYRVVSVLTVLHQDWL |
| | | NGKEYKCKVSNKALPAPIEKTISKAKGQPREPQVCTLPPSREE |
| | | MTKNQVSLSCAVKGFYPSDIAVEWESNGQPENNYKTTPPVL |
| | | DSDGSFFLVSKLTVDKSRWQQGNVFSCSVMHEALHNHYTQ |
| | | KSLSLSPGK |
| 32 | Fcₐ (hole) | PVAGPSVFLFPPKPKDTLMISRTPEVTCVVVDVKHEDPEVKF |
| | (RF) | NWYVDGVEVHNAKTKPREEQYNSTYRVVSVLTVLHQDWL |
| | | NGKEYKCKVSNKALPAPIEKTISKAKGQPREPQVCTLPPSREE |
| | | MTKNQVSLSCAVKGFYPSDIAVEWESNGQPENNYKTTPPVL |
| | | DSDGSFFLVSKLTVDKSRWQQGNVFSCSVMHEALHNRFTQK |
| | | SLSLSPGK |
| 33 | Fc_{b} (knob) | PVAGPSVFLFPPKPKDTLMISRTPEVTCVVVDVKHEDPEVKF |
| | | NWYVDGVEVHNAKTKPREEQYNSTYRVVSVLTVLHQDWL |
| | | NGKEYKCKVSNKALPAPIEKTISKAKGQPREPQVYTLPPCRE |
| | | EMTKNQVSLWCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| 34 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | | NHKPSNTKVDKRVEPKSCDKTGGSEVQLVESGGGLVQPGGS |
| | | LRLSCVASGDVHKINFLGWYRQAPGKEREKVAHISIGDQTD |
| | | YADSAKGRFTISRDESKNMVYLQMNSLKPEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 35 | Backbone | ASTKGPSVFPLAPSSKSTSGGTAALGCLVKDYFPEPVTVSWN |
| | (TCR/hole/RF | SGALTSGVHTFPAVLQSSGLYSLSSVVTVPSSSLGTQTYICNV |
| | | NHKPSNTKVDKRVEPKSCDKTGGSEVQLVESGGGLVQPGGS |
| | | LRLSCVASGDVHKINFLGWYRQAPGKEREKVAHISIGDQTD |
| | | YADSAKGRFTISRDESKNMVYLQMNSLKPEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 36 | Backbone | ASTKGPSVFPLAPSSKSTSGGTAALGCLVKDYFPEPVTVSWN |
| | (CD8/knob) | SGALTSGVHTFPAVLQSSGLYSLSSVVTVPSSSLGTQTYICNV |
| | | NHKPSNTKVDKRVEPKSCDKTGGSEVQLLESGGGLVQPGGS |
| | | LRLSCAASGFTFDDYAIGWFRQAPGKEREGVSCIRVSDGSTY |
| | | YADSVKGRFTISRDNSKNTLYLQMNSLRAEDTAVYYCAAGS |
| | | LYTCVQSIVWPARPYYDMDYWGQGTLVTVSSGGGGSTHTC |
| | | PPCPAPPVAGPSVFLFPPKPKDTLMISRTPEVTCVVVDVKHED |
| | | PEVKFNWYVDGVEVHNAKTKPREEQYNSTYRVVSVLTVLH |
| | | QDWLNGKEYKCKVSNKALPAPIEKTISKAKGQPREPQVYTL |
| | | PPCREEMTKNQVSLWCLVKGFYPSDIAVEWESNGQPENNYK |
| | | TTPPVLDSDGSFFLYSKLTVDKSRWQQGNVFSCSVMHEALH |
| | | NHYTQKSLSLSPGK |
| 37 | TCR V_{HHa} hu | INFLS |
| | 2.1, 2.3 | |
| | CDR1 | |
| 38 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.1 | NHKPSNTKVDKRVEPKSCDKTGGSEVQLLESGGGLVQPGGS |
| | V_{HHa} | LRLSCAASGDVHKINFLSWYRQAPGKEREKVAHISIGDQTDY |
| | | ADSAKGRFTISRDESKNTLYLQMNSLRAEDTAVYYCRAFSRI |
| | | YPYDYWGQGTLVTVSSGGGGSTHTCPPCPAPPVAGPSVFLFP |
| | | PKPKDTLMISRTPEVTCVVVDVKHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSREEMTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 39 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.2 | NHKPSNTKVDKRVEPKSCDKTGGSEVQLLESGGGLVQPGGS |
| | V_{HHa} | LRLSCAASGDVHKINFLGWYRQAPGKEREKVAHISIGDQTD |
| | | YADSAKGRFTISRDESKNTLYLQMNSLRAEDTAVYYCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 40 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.3 | NHKPSNTKVDKRVEPKSCDKTGGSEVQLLESGGGLVQPGGS |
| | V_{HHa} | LRLSCAASGDVHKINFLSWYRQAPGKGLEKVAHISIGDQTDY |
| | | ADSAKGRFTISRDESKNTLYLQMNSLRAEDTAVYYCRAFSRI |
| | | YPYDYWGQGTLVTVSSGGGGSTHTCPPCPAPPVAGPSVFLFP |
| | | PKPKDTLMISRTPEVTCVVVDVKHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSREEMTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 41 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.4 | NHKPSNTKVDKRVEPKSCDKTGGSEVQLLESGGGLVQPGGS |
| | V_{HHa} | LRLSCAASGDVHKINFLGWYRQAPGKGLEKVAHISIGDQTD |
| | | YADSAKGRFTISRDESKNTLYLQMNSLRAEDTAVYYCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 42 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.5 | NHKPSNTKVDKRVEPKSCDKTGGSEVQLLESGGGLVQPGGS |
| | V_{HHa} | LRLSCAASGDVHKINFLGWYRQAPGKEREKVAHISIGDQTD |
| | | YADSAKGRFTISRDESKNTVYLQMNSLRAEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 43 | Backbone | ASTKGPSVFPLAPSSKSTSGGTAALGCLVKDYFPEPVTVSWN |
| | (TCR/hole) | SGALTSGVHTFPAVLQSSGLYSLSSVVTVPSSSLGTQTYICNV |
| | TCR hu2.6 | NHKPSNTKVDKRVEPKSCDKTGGSEVQLLESGGGLVQPGGS |
| | V_{HHa} | LRLSCAASGDVHKINFLGWYRQAPGKGLEKVAHISIGDQTD |
| | | YADSAKGRFTISRDESKNTVYLQMNSLRAEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 44 | Backbone | ASTKGPSVFPLAPSSKSTSGGTAALGCLVKDYFPEPVTVSWN |
| | (TCR/hole/ | SGALTSGVHTFPAVLQSSGLYSLSSVVTVPSSSLGTQTYICNV |
| | RF) TCR hu2.1 | NHKPSNTKVDKRVEPKSCDKTGGSEVQLLESGGGLVQPGGS |
| | V_{HHa} | LRLSCAASGDVHKINFLSWYRQAPGKEREKVAHISIGDQTDY |
| | | ADSAKGRFTISRDESKNTLYLQMNSLRAEDTAVYYCRAFSRI |
| | | YPYDYWGQGTLVTVSSGGGGSTHTCPPCPAPPVAGPSVFLFP |
| | | PKPKDTLMISRTPEVTCVVVDVKHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSREEMTKNQVSLSC |
| | | AVKGFYPSDIAVEWESNGQPENNYKTTPPVLDSDGSFFLVSK |
| | | LTVDKSRWQQGNVFSCSVMHEALHNRFTQKSLSLSPGK |
| 45 | Backbone | ASTKGPSVFPLAPSSKSTSGGTAALGCLVKDYFPEPVTVSWN |
| | (TCR/hole/ | SGALTSGVHTFPAVLQSSGLYSLSSVVTVPSSSLGTQTYICNV |
| | RF) TCR hu2.2 | NHKPSNTKVDKRVEPKSCDKTGGSEVQLLESGGGLVQPGGS |
| | V_{HHa} | LRLSCAASGDVHKINFLGWYRQAPGKEREKVAHISIGDQTD |
| | | YADSAKGRFTISRDESKNTLYLQMNSLRAEDTAVYYCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 46 | Backbone | ASTKGPSVFPLAPSSKSTSGGTAALGCLVKDYFPEPVTVSWN |
| | (TCR/hole/ | SGALTSGVHTFPAVLQSSGLYSLSSVVTVPSSSLGTQTYICNV |
| | RF) TCR hu2.3 | NHKPSNTKVDKRVEPKSCDKTGGSEVQLLESGGGLVQPGGS |
| | V_{HHa} | LRLSCAASGDVHKINFLSWYRQAPGKGLEKVAHISIGDQTDY |
| | | ADSAKGRFTISRDESKNTLYLQMNSLRAEDTAVYYCRAFSRI |
| | | YPYDYWGQGTLVTVSSGGGGSTHTCPPCPAPPVAGPSVFLFP |
| | | PKPKDTLMISRTPEVTCVVVDVKHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSREEMTKNQVSLSC |
| | | AVKGFYPSDIAVEWESNGQPENNYKTTPPVLDSDGSFFLVSK |
| | | LTVDKSRWQQGNVFSCSVMHEALHNRFTQKSLSLSPGK |
| 47 | Backbone | ASTKGPSVFPLAPSSKSTSGGTAALGCLVKDYFPEPVTVSWN |
| | (TCR/hole/ | SGALTSGVHTFPAVLQSSGLYSLSSVVTVPSSSLGTQTYICNV |
| | RF) TCR hu2.4 | NHKPSNTKVDKRVEPKSCDKTGGSEVQLLESGGGLVQPGGS |
| | V_{HHa} | LRLSCAASGDVHKINFLGWYRQAPGKGLEKVAHISIGDQTD |
| | | YADSAKGRFTISRDESKNTLYLQMNSLRAEDTAVYYCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 48 | Backbone | ASTKGPSVFPLAPSSKSTSGGTAALGCLVKDYFPEPVTVSWN |
| | (TCR/hole/ | SGALTSGVHTFPAVLQSSGLYSLSSVVTVPSSSLGTQTYICNV |
| | RF) TCR hu2.5 | NHKPSNTKVDKRVEPKSCDKTGGSEVQLLESGGGLVQPGGS |
| | V_{HHa} | LRLSCAASGDVHKINFLGWYRQAPGKEREKVAHISIGDQTD |
| | | YADSAKGRFTISRDESKNTVYLQMNSLRAEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 49 | Backbone | ASTKGPSVFPLAPSSKSTSGGTAALGCLVKDYFPEPVTVSWN |
| | (TCR/hole/ | SGALTSGVHTFPAVLQSSGLYSLSSVVTVPSSSLGTQTYICNV |
| | RF) TCR hu2.6 | NHKPSNTKVDKRVEPKSCDKTGGSEVQLLESGGGLVQPGGS |
| | V_{HHa} | LRLSCAASGDVHKINFLGWYRQAPGKGLEKVAHISIGDQTD |
| | | YADSAKGRFTISRDESKNTVYLQMNSLRAEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 50 | CD8 V_{HHb} | EVQLLESGGGLVQPGGSLRLSCAASGFTFPDYAIGWFRQAPG |
| | D30P/S75A/ | KEREGVSCIRVSDGSTYYADSVKGRFTISRDNAKNTLYLQM |
| | Q106N | NSLRAEDTAVYYCAAGSLYTCVNSIVWPARPYYDMDYWGQ |
| | | GTLVTVSS |
| 51 | CD8 V_{HHb} | EVQLLESGGGLVQPGGSLRLSCAASGFTFPDYAIGWFRQAPG |
| | D30P/S75A/ | KEREGVSCIRVSDGSTYYADSVKGRFTISRDNAKNTLYLQM |
| | L101A | NSLRAEDTAVYYCAAGSAYTCVQSIVWPARPYYDMDYWG |
| | | QGTLVTVSS |
| 52 | CD8 V_{HHb} | EVQLLESGGGLVQPGGSLRLSCAASGFTFRDYAIGWFRQAPG |
| | S75A/D30R | KEREGVSCIRVSDGSTYYADSVKGRFTISRDNAKNTLYLQM |
| | | NSLRAEDTAVYYCAAGSLYTCVQSIVWPARPYYDMDYWGQ |
| | | GTLVTVSS |
| 53 | CD8 V_{HHb} | EVQLLESGGGLVQPGGSLRLSCAASGFTFPDYAIGWFRQAPG |
| | D30P/S75A/ | KEREGVSCIRVSDGSTYYADSVKGRFTISRDNAKNTLYLQM |
| | S100G | NSLRAEDTAVYYCAAGGLYTCVQSIVWPARPYYDMDYWG |
| | | QGTLVTVSS |
| 54 | CD8 V_{HHb} | EVQLLESGGGLVQPGGSLRLSCAASGFTFPDYAIGWFRQAPG |
| | D30P/S75A/Y | KEREGVSCIRVSDGSTYYADSVKGRFTISRDNAKNTLYLQM |
| | 102I | NSLRAEDTAVYYCAAGSLITCVQSIVWPARPYYDMDYWGQ |
| | | GTLVTVSS |
| 55 | CD8 V_{HHb} | EVQLLESGGGLVQPGGSLRLSCAASGFTFPDYAIGWFRQAPG |
| | D30P/S75A/S | KEREGVSCIRVSDGSTYYADSVKGRFTISRDNAKNTLYLQM |
| | 100P | NSLRAEDTAVYYCAAGPLYTCVQSIVWPARPYYDMDYWGQ |
| | | GTLVTVSS |
| 56 | CD8 V_{HHb} | EVQLLESGGGLVQPGGSLRLSCAASGFTFDSYAIGWFRQAPG |
| | S75A/D31S | KEREGVSCIRVSDGSTYYADSVKGRFTISRDNAKNTLYLQM |
| | | NSLRAEDTAVYYCAAGSLYTCVQSIVWPARPYYDMDYWGQ |
| | | GTLVTVSS |
| 57 | CD8 V_{HHb} | EVQLLESGGGLVQPGGSLRLSCAASGFTFPDYAIGWFRQAPG |
| | D30P/S75A/R | KEREGVSCITVSDGSTYYADSVKGRFTISRDNAKNTLYLQM |
| | 52T | NSLRAEDTAVYYCAAGSLYTCVQSIVWPARPYYDMDYWGQ |
| | | GTLVTVSS |
| 58 | CD8 V_{HHb} | EVQLLESGGGLVQPGGSLRLSCAASGFTFPDYAIGWFRQAPG |
| | D30P/S75A | KEREGVSCIRVSDGSTYYADSVKGRFTISRDNAKNTLYLQM |
| | | NSLRAEDTAVYYCAAGSLYTCVQSIVWPARPYYDMDYWGQ |
| | | GTLVTVSS |
| 59 | IgG1 IMGT | EPKSCDK |
| | Upper Hinge | |
| | Region | |
| 60 | IMGT Lower | THTCPPCP |
| | Hinge Region | |
| 61 | WT C_{H1} | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKRVEPKSC |
| 62 | WT C_{H1} | ASTKGPSVFPLAPSSKSTSGGTAALGCLVKDYFPEPVTVSW |
| | (IMGT) | NSGALTSGVHTFPAVLQSSGLYSLSSVVTVPSSSLGTQTYIC |
| | | NVNHKPSNTKVDKRV |
| 63 | V12 C_{H1} | ASTKGPSVCPLAPSSKSTSGGTAALGCLVKDYFPEPVTVSW |
| | (F126C/ | NSGALTSGVHTFPAVLQSSGLYSLSSVVTVPSSSLGTQTYIC |
| | C220V) | NVNHKPSNTKVDKRVEPKSV |
| 64 | WT CLλ | GQPKAAPSVTLFPPSSEELQANKATLVCLISDFYPGAVTVAW |
| | | KADSSPVKAGVETTTPSKQSNNKYAASSYLSLTPEQWKSHR |
| | | SYSCQVTHEGSTVEKTVAPTECS |
| 65 | V12 LC | GQPKAAPSVTLFPPCSEELQANKATLVCLISDFYPGAVTVAW |
| | lambda | KADSSPVKAGVETTTPSKQSNNKYAASSYLSLTPEQWKSHR |
| | constant | SYSCQVTHEGSTVEKTVAPTEVS |
| | (CLλ) | |
| 66 | WT LC kappa | RTVAAPSVFIFPPSDEQLKSGTASVVCLLNNFYPREAKVQWK |
| | constant (Cκ) | VDNALQSGNSQESVTEQDSKDSTYSLSSTLTLSKADYEKHK |
| | region | VYACEVTHQGLSSPVTKSFNRGEC |
| 67 | IgG1 CH2 | ELLGGPSVFLFPPKPKDTLMISRTPEVTCVVVDVSHEDPEVKF |
| | Wild-type | NWYVDGVEVHNAKTKPREEQYNSTYRVVSVLTVLHQDWL |
| | | NGKEYKCKVSNKALPAPIEKTIS |
| 68 | IgG1 CH2 Fc- | PVAGPSVFLFPPKPKDTLMISRTPEVTCVVVDVKHEDPEVKF |
| | null | NWYVDGVEVHNAKTKPREEQYNSTYRVVSVLTVLHQDWL |
| | | NGKEYKCKVSNKALPAPIEKTIS |
| 69 | IgG1 CH3 | GQPREPQVYTLPPSREEMTKNQVSLTCLVKGFYPSDIAVEWE |
| | Wild-type | SNGQPENNYKTTPPVLDSDGSFFLYSKLTVDKSRWQQGNVF |
| | | SCSVMHEALHNHYTQKSLSLSPGK |
| 70 | C_{H1} with | ASTKGPSVFPLAPSSKSTSGGTASLGCLVKDYFPEPVTVSW |
| | A141S | NSGALTSGVHTFPAVLQSSGLYSLSSVVTVPSSSLGTQTYIC |
| | | NVNHKPSNTKVDKRVEPKSC |
| 71 | CLλ with | GQPKAAPSVRLFPPSSEELQANKATLVCLISDFYPGAVTVAW |
| | T117R | KADSSPVKAGVETTTPSKQSNNKYAASSYLSLTPEQWKSHR |
| | | SYSCQVTHEGSTVEKTVAPTECS |
| 72 | V12 C_{H1} with | ASTKGPSVCPLAPSSKSTSGGTASLGCLVKDYFPEPVTVSW |
| | A141S | NSGALTSGVHTFPAVLQSSGLYSLSSVVTVPSSSLGTQTYIC |
| | | NVNHKPSNTKVDKRVEPKSV |
| 73 | V12 CLλ with | GQPKAAPSVRLFPPCSEELQANKATLVCLISDFYPGAVTVAW |
| | T117R | KADSSPVKAGVETTTPSKQSNNKYAASSYLSLTPEQWKSHR |
| | | SYSCQVTHEGSTVEKTVAPTEVS |
| 74 | IgG1 CH2 | APELLGGPSVFLFPPKPKDTLMISRTPEVTCVVVDVSHEDPEV |
| | WT(IMGT) | KFNWYVDGVEVHNAKTKPREEQYNSTYRVVSVLTVLHQD |
| | | WLNGKEYKCKVSNKALPAPIEKTIS |
| 75 | IgG1 CH2 Fc- | APPVAGPSVFLFPPKPKDTLMISRTPEVTCVVVDVKHEDPEV |
| | null (IMGT) | KFNWYVDGVEVHNAKTKPREEQYNSTYRVVSVLTVLHQD |
| | | WLNGKEYKCKVSNKALPAPIEKTIS |
| 76 | TPP- | SYSMN |
| | 47648/TPP- | |
| | 47651/TPP- | |
| | 47645 BCMA | |
| | VH CDR1 | |
| 77 | TPP-47648 | SISGQSNYIYYADSVKG |
| | /TPP-47651 | |
| | TPP-47645 | |
| | BCMA | |
| | VH CDR2 | |
| 78 | TPP-47648 | GGNYFVEYFQY |
| | /TPP-47651 | |
| | TPP-47645 | |
| | BCMA VH | |
| | CDR3 | |
| 79 | TPP-47648 | RASQYISSNNLA |
| | /TPP-47651 | |
| | TPP-47645 | |
| | BCMA VL | |
| | CDR1 | |
| 80 | TPP-47648 | GASNRAT |
| | /TPP-47651 | |
| | TPP-47645 | |
| | BCMA VL | |
| | CDR2 | |
| 81 | TPP-47648 | QQYADSPIT |
| | /TPP-47651 | |
| | TPP-47645 | |
| | BCMA VL | |
| | CDR3 | |
| 82 | TPP-47648 | EVQLVESGGGLVKPGGSLRLSCAASGFTFSSYSMNWVRQAP |
| | /TPP-47651 | GKGLEWVSSISGQSNYIYYADSVKGRFTISRDNAKNSLYLQM |
| | BCMA VH1 | NSLRAEDTAVYYCARGGNYFVEYFQYWGQGTLVTVSS |
| 83 | TPP-47648 | EIVLTQSPGTLSLSPGERATLSCRASQYISSNNLAWYQQKPGQ |
| | /TPP-47651 | APRLLIYGASNRATGIPDRFSGSGSGTDFTLTISRLEPEDFAVY |
| | BCMA VL1 | YCQQYADSPITFGQGTKLEIK |
| 84 | TPP-47648 | EIVLTQSPGTLSLSPGERATLSCRASQYISSNNLAWYQQKPGQ |
| | BCMA Light | APRLLIYGASNRATGIPDRFSGSGSGTDFTLTISRLEPEDFAVY |
| | Chain | YCQQYADSPITFGQGTKLEIKRTVAAPSVFIFPPSDEQLKSGT |
| | | ASVECLLNNFYPREAKVQWKVDNALQSGNSQESVTEQDSK |
| | | DSTYSLSSTLTLSKADYEKHKVYACEVTHQGLSSPVTKSFNR |
| | | GEC |
| 85 | TPP-47648 | EVQLVESGGGLVKPGGSLRLSCAASGFTFSSYSMNWVRQAP |
| | /TPP-47651 | GKGLEWVSSISGQSNYIYYADSVKGRFTISRDNAKNSLYLQM |
| | BCMA VH1- | NSLRAEDTAVYYCARGGNYFVEYFQYWGQGTLVTVSSAST |
| | CH1 | KGPSVFPLAPSSKSTSGGTAALGCLVKDYFPEPVTVSWNSGA |
| | | LTSGVHTFPAVLQSSGLYSLKSVVTVPSSSLGTQTYICNVNH |
| | | KPSNTKVDKRV |
| 86 | TPP-47648- | EVQLVESGGGLVKPGGSLRLSCAASGFTFSSYSMNWVRQAP |
| | hole (TCR) | GKGLEWVSSISGQSNYIYYADSVKGRFTISRDNAKNSLYLQM |
| | BCMA HC | NSLRAEDTAVYYCARGGNYFVEYFQYWGQGTLVTVSSAST |
| | TCR (FC | KGPSVFPLAPSSKSTSGGTAALGCLVKDYFPEPVTVSWNSGA |
| | hole) | LTSGVHTFPAVLQSSGLYSLKSVVTVPSSSLGTQTYICNVNH |
| | | KPSNTKVDKRVEPKSCDKTGGSEVQLVESGGGLVQPGGSLR |
| | | LSCVASGDVHKINFLGWYRQAPGKEREKVAHISIGDQTDYA |
| | | DSAKGRFTISRDESKNMVYLQMNSLKPEDTAVYFCRAFSRIY |
| | | PYDYWGQGTLVTVSSGGGGSTHTCPPCPAPPVAGPSVFLFPP |
| | | KPKDTLMISRTPEVTCVVVDVKHEDPEVKFNWYVDGVEVH |
| | | NAKTKPREEQYNSTYRVVSVLTVLHQDWLNGKEYKCKVSN |
| | | KALPAPIEKTISKAKGQPREPQVCTLPPSREEMTKNQVSLSCA |
| | | VKGFYPSDIAVEWESNGQPENNYKTTPPVLDSDGSFFLVSKL |
| | | TVDKSRWQQGNVFSCSVMHEALHNRFTQKSLSLSPGK |
| 88 | TPP-47651 | EIVLTQSPGTLSLSPGERATLSCRASQYISSNNLAWYQQKPGQ |
| | BCMA Light | APRLLIYGASNRATGIPDRFSGSGSGTDFTLTISRLEPEDFAVY |
| | Chain | YCQQYADSPITFGQGTKLEIKGQPKAAPSVRLFPPCSEELQAN |
| | | KATLVCLISDFYPGAVTVAWKADSSPVKAGVETTTPSKQSN |
| | | NKYAASSYLSLTPEQWKSHRSYSCQVTHEGSTVEKTVAPTE |
| | | VS |
| 89 | TPP-47651 | DIQMTQSPSSLSASVGDRVTITCRASSSVSYMHWYQQKPGK |
| | CD20 Light | APKPLIYAPSNLASGVPSRFSGSGSGTDFTLTISSLQPEDFATY |
| | Chain | YCQQWSFNPPTFGQGTKVEIKRTVAAPSVFIFPPSDEQLKSGT |
| | | ASVECLLNNFYPREAKVQWKVDNALQSGNSQESVTEQDSK |
| | | DSTYSLSSTLTLSKADYEKHKVYACEVTHQGLSSPVTKSFNR |
| | | GEC |
| 90 | TPP-47651 | EVQLVESGGGLVKPGGSLRLSCAASGFTFSSYSMNWVRQAP |
| | knob (CD8) | GKGLEWVSSISGQSNYIYYADSVKGRFTISRDNAKNSLYLQM |
| | BCMA HC | NSLRAEDTAVYYCARGGNYFVEYFQYWGQGTLVTVSSAST |
| | CD8 (FC | KGPSVCPLAPSSKSTSGGTADLGCLVKDYFPEPVTVSWNSGA |
| | knob) | LTSGVHTFPAVLQSSGLYSLSSVVTVPSSSLGTQTYICNVNHK |
| | | PSNTKVDKRVEPKSVDKTGGSEVQLLESGGGLVQPGGSLRL |
| | | SCAASGFTFTDYAIGWFRQAPGKEREGVSCIRVSDGSTYYAD |
| | | SVKGRFTISRDSSKNTLYLQMNSLRAEDTAVYYCAAGSLYT |
| | | CVQSIVWPARPYYDMDYWGQGTLVTVSSGGGGSTHTCPPCP |
| | | APPVAGPSVFLFPPKPKDTLMISRTPEVTCVVVDVKHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPPC |
| | | REEMTKNQVSLWCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| 91 | TPP-47651- | EVQLVESGGGLVQPGGSLRLSCAASGYTFTSYNMHWVRQA |
| | hole (TCR) | PGKGLEWVGAIYPGSGDTSYNQKFKGRFTISVDKSKNTLYL |
| | CD20 HC | QMNSLRAEDTAVYYCARVVYYSNSYWYFDVWGQGTLVTV |
| | TCR (FC | SSASTKGPSVFPLAPSSKSTSGGTAALGCLVKDYFPEPVTVS |
| | hole) | WNSGALTSGVHTFPAVLQSSGLYSLKSVVTVPSSSLGTQTYI |
| | | CNVNHKPSNTKVDKRVEPKSCDKTGGSEVQLVESGGGLVQP |
| | | GGSLRLSCVASGDVHKINFLGWYRQAPGKEREKVAHISIGD |
| | | QTDYADSAKGRFTISRDESKNMVYLQMNSLKPEDTAVYFCR |
| | | AFSRIYPYDYWGQGTLVTVSSGGGGSTHTCPPCPAPPVAGPS |
| | | VFLFPPKPKDTLMISRTPEVTCVVVDVKHEDPEVKFNWYVD |
| | | GVEVHNAKTKPREEQYNSTYRVVSVLTVLHQDWLNGKEYK |
| | | CKVSNKALPAPIEKTISKAKGQPREPQVCTLPPSREEMTKNQ |
| | | VSLSCAVKGFYPSDIAVEWESNGQPENNYKTTPPVLDSDGSF |
| | | FLVSKLTVDKSRWQQGNVFSCSVMHEALHNRFTQKSLSLSP |
| | | GK |
| 92 | TPP-47646 | EVQLVESGGGLVKPGGSLRLSCAASGFTFSSYSMNWVRQAP |
| | BCMA VH | GKGLEWVSSISGQSNYIYYADSVKGRFTISRDNAKNSLYLQM |
| | | NSLRAEDTAVYYCARGGNYFVEYFQYWGQGTLVTVSS |
| 93 | TPP-47646 | EIVLTQSPGTLSLSPGERATLSCRASQYISSNNLAWYQQKPGQ |
| | BCMA VL | APRLLIYGASNRATGIPDRFSGSGSGTDFTLTISRLEPEDFAVY |
| | | YCQQYADSPITFGQGTKLEIK |
| 94 | TPP-47646 | EIVLTQSPGTLSLSPGERATLSCRASQYISSNNLAWYQQKPGQ |
| | BCMA Light | APRLLIYGASNRATGIPDRFSGSGSGTDFTLTISRLEPEDFAVY |
| | Chain VL-CL | YCQQYADSPITFGQGTKLEIKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 95 | TPP-47646 | EVQLVESGGGLVKPGGSLRLSCAASGFTFSSYSMNWVRQAP |
| | BCMA VH- | GKGLEWVSSISGQSNYIYYADSVKGRFTISRDNAKNSLYLQM |
| | CH1 | NSLRAEDTAVYYCARGGNYFVEYFQYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSC |
| 96 | TPP-47646 | EVQLVESGGGLVKPGGSLRLSCAASGFTFSSYSMNWVRQAP |
| | knob (CD8) | GKGLEWVSSISGQSNYIYYADSVKGRFTISRDNAKNSLYLQM |
| | BCMA HC | NSLRAEDTAVYYCARGGNYFVEYFQYWGQGTLVTVSSAST |
| | CD8 (FC | KGPSVFPLAPSSKSTSGGTAALGCLVKDYFPEPVTVSWNSGA |
| | knob) | LTSGVHTFPAVLQSSGLYSLSSVVTVPSSSLGTQTYICNVNHK |
| | | PSNTKVDKRVEPKSCDKTGGSEVQLLESGGGLVQPGGSLRLS |
| | | CAASGFTFTDYAIGWFRQAPGKEREGVSCIRVSDGSTYYADS |
| | | VKGRFTISRDSSKNTLYLQMNSLRAEDTAVYYCAAGSLYTC |
| | | VQSIVWPARPYYDMDYWGQGTLVTVSSGGGGSTHTCPPCP |
| | | APPVAGPSVFLFPPKPKDTLMISRTPEVTCVVVDVKHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPPC |
| | | REEMTKNQVSLWCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| 97 | TPP-47646 | EVQLVESGGGLVKPGGSLRLSCAASGFTFSSYSMNWVRQAP |
| | hole (TCR) | GKGLEWVSSISGQSNYIYYADSVKGRFTISRDNAKNSLYLQM |
| | BCMA HC | NSLRAEDTAVYYCARGGNYFVEYFQYWGQGTLVTVSSAST |
| | TCR (FC | KGPSVFPLAPSSKSTSGGTAALGCLVKDYFPEPVTVSWNSGA |
| | hole) | LTSGVHTFPAVLQSSGLYSLSSVVTVPSSSLGTQTYICNVNHK |
| | | PSNTKVDKRVEPKSCDKTGGSEVQLVESGGGLVQPGGSLRL |
| | | SCVASGDVHKINFLGWYRQAPGKEREKVAHISIGDQTDYAD |
| | | SAKGRFTISRDESKNMVYLQMNSLKPEDTAVYFCRAFSRIYP |
| | | YDYWGQGTLVTVSSGGGGSTHTCPPCPAPPVAGPSVFLFPPK |
| | | PKDTLMISRTPEVTCVVVDVKHEDPEVKFNWYVDGVEVHN |
| | | AKTKPREEQYNSTYRVVSVLTVLHQDWLNGKEYKCKVSNK |
| | | ALPAPIEKTISKAKGQPREPQVCTLPPSREEMTKNQVSLSCAV |
| | | KGFYPSDIAVEWESNGQPENNYKTTPPVLDSDGSFFLVSKLT |
| | | VDKSRWQQGNVFSCSVMHEALHNRFTQKSLSLSPGK |
| 98 | TPP-47645 | EVQLVESGGGLVQPGGSLRLSCAASGYTFTSYNMHWVRQA |
| | CD20 VH | PGKGLEWVGAIYPGSGDTSYNQKFKGRFTISVDKSKNTLYL |
| | | QMNSLRAEDTAVYYCARVVYYSNSYWYFDVWGQGTLVTV |
| | | SS |
| 99 | TPP-47645 | DIQMTQSPSSLSASVGDRVTITCRASSSVSYMHWYQQKPGK |
| | CD20 VL | APKPLIYAPSNLASGVPSRFSGSGSGTDFTLTISSLQPEDFATY |
| | | YCQQWSFNPPTFGQGTKVEIK |
| 100 | TPP-47645 | DIQMTQSPSSLSASVGDRVTITCRASSSVSYMHWYQQKPGK |
| | CD20 Light | APKPLIYAPSNLASGVPSRFSGSGSGTDFTLTISSLQPEDFATY |
| | Chain VL-CL | YCQQWSFNPPTFGQGTKVEIKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 101 | TPP-47645 | EVQLVESGGGLVQPGGSLRLSCAASGYTFTSYNMHWVRQA |
| | CD20 VH- | PGKGLEWVGAIYPGSGDTSYNQKFKGRFTISVDKSKNTLYL |
| | CH1 | QMNSLRAEDTAVYYCARVVYYSNSYWYFDVWGQGTLVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKRVEPKSC |
| 102 | TPP-47645 | EVQLVESGGGLVQPGGSLRLSCAASGYTFTSYNMHWVRQA |
| | knob (CD8) | PGKGLEWVGAIYPGSGDTSYNQKFKGRFTISVDKSKNTLYL |
| | CD20 HC | QMNSLRAEDTAVYYCARVVYYSNSYWYFDVWGQGTLVTV |
| | CD8 (FC | SSASTKGPSVFPLAPSSKSTSGGTAALGCLVKDYFPEPVTVS |
| | knob) | WNSGALTSGVHTFPAVLQSSGLYSLSSVVTVPSSSLGTQTYIC |
| | | NVNHKPSNTKVDKRVEPKSCDKTGGSEVQLLESGGGLVQPG |
| | | GSLRLSCAASGFTFTDYAIGWFRQAPGKEREGVSCIRVSDGS |
| | | TYYADSVKGRFTISRDSSKNTLYLQMNSLRAEDTAVYYCAA |
| | | GSLYTCVQSIVWPARPYYDMDYWGQGTLVTVSSGGGGSTH |
| | | TCPPCPAPPVAGPSVFLFPPKPKDTLMISRTPEVTCVVVDVKH |
| | | EDPEVKFNWYVDGVEVHNAKTKPREEQYNSTYRVVSVLTV |
| | | LHQDWLNGKEYKCKVSNKALPAPIEKTISKAKGQPREPQVY |
| | | TLPPCREEMTKNQVSLWCLVKGFYPSDIAVEWESNGQPENN |
| | | YKTTPPVLDSDGSFFLYSKLTVDKSRWQQGNVFSCSVMHEA |
| | | LHNHYTQKSLSLSPGK |
| 103 | TPP-47645 | EVQLVESGGGLVQPGGSLRLSCAASGYTFTSYNMHWVRQA |
| | hole (TCR) | PGKGLEWVGAIYPGSGDTSYNQKFKGRFTISVDKSKNTLYL |
| | CD20 HC | QMNSLRAEDTAVYYCARVVYYSNSYWYFDVWGQGTLVTV |
| | TCR (FC | SSASTKGPSVFPLAPSSKSTSGGTAALGCLVKDYFPEPVTVS |
| | hole) | WNSGALTSGVHTFPAVLQSSGLYSLSSVVTVPSSSLGTQTYIC |
| | | NVNHKPSNTKVDKRVEPKSCDKTGGSEVQLVESGGGLVQPG |
| | | GSLRLSCVASGDVHKINFLGWYRQAPGKEREKVAHISIGDQT |
| | | DYADSAKGRFTISRDESKNMVYLQMNSLKPEDTAVYFCRAF |
| | | SRIYPYDYWGQGTLVTVSSGGGGSTHTCPPCPAPPVAGPSVF |
| | | LFPPKPKDTLMISRTPEVTCVVVDVKHEDPEVKFNWYVDGV |
| | | EVHNAKTKPREEQYNSTYRVVSVLTVLHQDWLNGKEYKCK |
| | | VSNKALPAPIEKTISKAKGQPREPQVCTLPPSREEMTKNQVSL |
| | | SCAVKGFYPSDIAVEWESNGQPENNYKTTPPVLDSDGSFFLV |
| | | SKLTVDKSRWQQGNVFSCSVMHEALHNHYTQKSLSLSPGK |
| 104 | CD8 V_{HHb} | EVQLLESGGGLVQPGGSLRLSCAASGFTFTDYAIGWFRQAPG |
| | D30T/N74S | KEREGVSCIRVSDGSTYYADSVKGRFTISRDSSKNTLYLQMN |
| | | SLRAEDTAVYYCAAGSLYTCVQSIVWPARPYYDMDYWGQG |
| | | TLVTVSS |
| 105 | CD8 V_{HHb} | GFTFTDYA |
| | D30T/N74S | |
| | CDR1 | |
| | IMGT | |
| 106 | CD8 V_{HHb} | IRVSDGST |
| | D30T/N74S | |
| | CDR2 | |
| | IMGT | |
| 107 | CD8 V_{HHb} | AAGSLYTCVQSIVWPARPYYDMDY |
| | D30T/N74S | |
| | CDR3 | |
| | IMGT | |
| 108 | TCR V_{HHa} | DVQLVESGGGVVQPGGSLRLSCVASGYVHKINFYGWYRQA |
| | alternative | PGKEREKVAHISIGDQTDYADSAKGRFTISRDESKNTVYL |
| | | QMNSLRPEDTAAYYCRALSRIWPYDYWGQGTLVTVSS |
| 109 | TCR V_{HHa} | INFYG |
| | CDR1 | |
| | alternative | |
| 110 | TCR V_{HHa} | HISIGDQTDYADSAKG |
| | CDR2 | |
| | alternative | |
| 111 | TCR V_{HHa} | LSRIWPYDY |
| | CDR3 | |
| | alternative | |
| 112 | Comparator T | EVQLVESGGGLVQPGGSLRLSCAASGFTFNTYAMNWVRQA |
| | HC (CD3) | PGKGLEWVARIRSKYNNYATYYAASVKGRFTISRDDSKNSL |
| | | YLQMNSLKTEDTAVYYCARHGNFGNSYVSWFAYWGQGTLV |
| | | TVSSASTKGPSVFPLAPCSRSTSESTAALGCLVKDYFPEPVT |
| | | VSWNSGALTSGVHTFPAVLQSSGLYSLSSVVTVPSSSLGTKT |
| | | YTCNVDHKPSNTKVDKRVESKYGPPCPPCPAPEAAGGPSVFL |
| | | FPPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQVSLT |
| | | CLVKGFYPSDIAVEWESNGQPENNYKTTPPVLDSDGSFLLYS |
| | | KLTVDKSRWQEGNVFSCSVMHEALHNHYTQKSLSLSLGK |
| 113 | Comparator T | QTVVTQEPSLTVSPGGTVTLTCRSSTGAVTTSNYANWVQQK |
| | LC (CD3) | PGQAPRGLIGGTNKRAPGTPARFSGSLLGGKAALTLSGVQPE |
| | | DEAEYYCALWYSNLWVFGGGTKLTVLGQPKAAPSVTLFPPS |
| | | SEELQANKATLVCLISDFYPGAVTVAWKADSSPVKAGVETT |
| | | TPSKQSNNKYAASSYLSLTPEQWKSHRSYSCQVTHEGSTVE |
| | | KTVAPTECS |
| 114 | Comparator T | QLQLQESGPGLVKPSETLSLTCTVSGGSISSGSYFWGWIRQPP |
| | HC (BCMA) | GKGLEWIGSIYYSGITYYNPSLKSRVTISVDTSKNQFSLKLSS |
| | | VTAADTAVYYCARHDGAVAGLFDYWGQGTLVTVSSASTKG |
| | | PSVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEAAGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEK |
| | | TISKAKGQPREPQVYTLPPSQEEMTKNQVSLTCLVKGFYPSDI |
| | | AVEWESNGQPENNYKTTPPVLDSDGSFFLYSRLTVDKSRWQ |
| | | EGNVFSCSVMHEALHNHYTQKSLSLSLGK |
| 115 | Comparator T | SYVLTQPPSVSVAPGQTARITCGGNNIGSKSVHWYQQPPGQA |
| | LC (BCMA) | PVVVVYDDSDRPSGIPERFSGSNSGNTATLTISRVEAGDEAV |
| | | YYCQVWDSSSDHVVFGGGTKLTVLGQPKAAPSVTLFPPSSE |
| | | ELQANKATLVCLISDFYPGAVTVAWKGDSSPVKAGVETTTP |
| | | SKQSNNKYAASSYLSLTPEQWKSHRSYSCQVTHEGSTVEKT |
| | | VAPTECS |
| 116 | IgG1 IMGT | EPKSVDK |
| | Upper Hinge | |
| | Region | |
| 117 | CD8 V_{HHb} | CIRVSDGSTYYADSVKG |
| | CDR2 | |
| | (alternate) | |
| | TPP-47648 | |
| 118 | CD20 | GAGGTGCAGCTGGTGGAATCCGGTGGGGGTCTGGTACAG |
| | Heavy Chain | CCTGGGGGTTCACTGAGATTGTCATGCGCAGCCTCCGGGT |
| | Knob (CD8) | ACACATTCACATCATACAATATGCACTGGGTGAGGCAAGC |
| | | TCCTGGCAAAGGGTTGGAATGGGTAGGAGCAATTTATCCT |
| | | GGCAGCGGCGACACCTCATATAACCAAAAATTTAAAGGTC |
| | | GGTTTACCATAAGTGTCGACAAATCCAAAAACACCTTGTA |
| | | TTTGCAGATGAACTCACTGCGAGCTGAAGACACAGCAGTC |
| | | TATTACTGTGCTCGTGTAGTATATTACTCAAATTCCTACTG |
| | | GTATTTTGATGTATGGGGGCAGGGCACTCTCGTGACTGTA |
| | | TCAAGCGCAAGTACAAAGGGACCTTCTGTGTGTCCTCTGG |
| | | CTCCCTCCAGCAAGTCTACCTCTGGTGGAACAGCTGACCT |
| | | GGGCTGCCTGGTCAAGGATTACTTTCCTGAGCCTGTGACC |
| | | GTGTCCTGGAACTCTGGCGCTCTGACATCTGGCGTGCACA |
| | | CCTTTCCAGCTGTGCTGCAGTCCTCCGGCCTGTACTCTCTG |
| | | TCCTCTGTCGTGACCGTGCCTTCCAGCTCTCTGGGCACCCA |
| | | GACCTACATCTGCAATGTGAACCACAAGCCTTCCAACACC |
| | | AAGGTGGACAAGAGAGTGGAACCAAAAAGCGTGGACAAG |
| | | ACCGGAGGATCTGAGGTACAGCTGCTGGAATCAGGAGGC |
| | | GGCTTGGTTCAGCCAGGAGGCTCACTTCGGCTCTCCTGCG |
| | | CAGCAAGTGGGTTTACATTTACTGATTATGCTATAGGCTG |
| | | GTTTCGGCAAGCACCTGGTAAGGAACGCGAAGGTGTGTCA |
| | | TGTATTCGCGTAAGCGACGGATCAACATACTATGCAGACA |
| | | GTGTCAAAGGTAGATTTACTATTAGTCGTGACAGCTCAAA |
| | | GAATACTCTGTACTTGCAAATGAATTCCCTTCGTGCCGAA |
| | | GATACTGCCGTTTACTATTGCGCCGCAGGATCACTCTACA |
| | | CCTGCGTGCAGAGCATTGTTTGGCCTGCCAGGCCTTATTAT |
| | | GATATGGACTATTGGGGCCAAGGCACTTTGGTCACTGTTA |
| | | GCTCTGGTGGCGGAGGATCTACCCACACCTGTCCTCCATG |
| | | CCCTGCTCCACCCGTGGCCGGCCCATCCGTGTTTCTGTTCC |
| | | CTCCAAAGCCTAAGGACACCCTGATGATCTCTCGGACCCC |
| | | TGAAGTGACCTGCGTGGTGGTGGATGTGAAGCACGAGGA |
| | | TCCCGAAGTGAAGTTCAATTGGTACGTGGACGGCGTGGAA |
| | | GTGCACAACGCCAAGACCAAGCCTAGAGAGGAACAGTAC |
| | | AACTCCACCTACAGAGTGGTGTCCGTGCTGACCGTGCTGC |
| | | ACCAGGATTGGCTGAACGGCAAAGAGTACAAGTGCAAGG |
| | | TGTCCAACAAGGCCCTGCCTGCCCCCATCGAAAAGACCAT |
| | | CTCCAAGGCCAAGGGCCAGCCTAGGGAACCCCAGGTTTAC |
| | | ACCCTGCCACCTTGCCGGGAAGAGATGACCAAGAACCAG |
| | | GTGTCCCTGTGGTGCCTCGTGAAGGGCTTCTACCCTTCCGA |
| | | TATCGCCGTGGAATGGGAGAGCAATGGCCAGCCTGAGAA |
| | | CAACTACAAGACAACCCCTCCTGTGCTGGACTCCGACGGC |
| | | TCATTCTTCCTGTACTCCAAGCTGACAGTGGACAAGTCCA |
| | | GATGGCAGCAGGGCAACGTGTTCTCCTGCTCCGTGATGCA |
| | | CGAGGCCCTGCACAATCACTACACCCAGAAGTCCCTGTCT |
| | | CTGTCCCCTGGAAAA |
| 119 | TPP-47648 | GAGGTGCAGCTGGTGGAAAGTGGAGGTGGCCTGGTTAAG |
| | BCMA | CCTGGCGGGAGCCTGCGCCTGAGCTGCGCTGCCAGCGGCT |
| | Heavy Chain | TCACTTTCTCATCTTACAGCATGAACTGGGTGCGCCAGGC |
| | Hole (TCR) | CCCTGGCAAAGGCCTTGAGTGGGTTAGTAGCATCAGCGGC |
| | | CAGTCAAACTACATTTATTACGCCGATTCCGTCAAGGGCA |
| | | GGTTTACAATCTCCCGTGATAATGCGAAGAATAGCCTCTA |
| | | TCTCCAGATGAATTCCCTGCGCGCCGAAGATACAGCCGTG |
| | | TATTACTGCGCGAGGGGGGGGAATTACTTTGTTGAATACT |
| | | TCCAGTACTGGGGCCAGGGTACCCTCGTCACCGTTTCCAG |
| | | TGCAAGTACAAAGGGACCTTCTGTGTTCCCTCTGGCTCCCT |
| | | CCAGCAAGTCTACATCTGGTGGAACCGCTGCTCTGGGCTG |
| | | CCTGGTCAAGGATTACTTTCCTGAGCCTGTGACCGTGTCCT |
| | | GGAATTCTGGTGCTCTGACCTCCGGCGTGCACACCTTTCC |
| | | AGCTGTGCTGCAGTCCTCTGGCCTGTACTCTCTGAAGTCTG |
| | | TCGTGACCGTGCCTTCTAGCTCTCTGGGCACCCAGACCTA |
| | | CATCTGCAACGTGAACCACAAGCCTTCCAACACCAAGGTG |
| | | GACAAGAGAGTGGAACCAAAAAGCTGCGACAAGACCGGA |
| | | GGATCTGAAGTGCAGCTGGTCGAGTCTGGCGGAGGATTGG |
| | | TTCAACCTGGCGGCTCCCTGAGACTGTCTTGTGTGGCTAG |
| | | CGGAGATGTGCATAAGATCAATTTTCTCGGCTGGTACAGA |
| | | CAGGCCCCTGGCAAAGAGAGAGAGAAGGTCGCCCACATC |
| | | TCCATCGGCGACCAGACCGATTACGCCGACTCTGCCAAGG |
| | | GCAGATTCACCATCTCTCGGGACGAGTCCAAGAACATGGT |
| | | GTACCTGCAGATGAACTCCCTGAAGCCTGAGGATACCGCC |
| | | GTGTACTTCTGCCGGGCCTTCTCTCGGATCTACCCCTACGA |
| | | TTATTGGGGCCAGGGCACCCTGGTCACAGTGTCATCTGGT |
| | | GGCGGAGGATCTACCCACACCTGTCCTCCATGCCCTGCTC |
| | | CACCCGTGGCCGGCCCTTCCGTGTTCCTGTTTCCTCCAAAG |
| | | CCTAAGGACACCCTGATGATCTCTCGGACCCCTGAAGTGA |
| | | CCTGCGTGGTGGTGGATGTGAAGCACGAGGATCCCGAAGT |
| | | GAAGTTCAATTGGTACGTGGACGGCGTGGAAGTGCACAA |
| | | CGCCAAGACCAAGCCTAGAGAGGAACAGTACAACTCCAC |
| | | CTACAGAGTGGTGTCCGTGCTGACCGTGCTGCACCAGGAT |
| | | TGGCTGAACGGCAAAGAGTACAAGTGCAAGGTGTCCAAC |
| | | AAGGCCCTGCCTGCCCCCATCGAAAAGACCATCTCTAAGG |
| | | CTAAGGGCCAGCCTCGCGAGCCTCAAGTCTGTACACTGCC |
| | | TCCTAGCCGGGAAGAGATGACCAAGAACCAGGTGTCCCT |
| | | GTCTTGCGCCGTGAAGGGCTTCTACCCTTCCGATATCGCC |
| | | GTGGAATGGGAGTCCAATGGCCAGCCTGAGAACAACTAC |
| | | AAGACAACCCCTCCTGTGCTGGACTCCGACGGCTCATTCT |
| | | TCCTGGTGTCCAAGCTGACAGTGGACAAGTCCAGATGGCA |
| | | GCAGGGCAACGTGTTCTCCTGCTCTGTGATGCACGAGGCC |
| | | CTGCACAACCGGTTCACCCAGAAATCCCTGTCTCTGTCCC |
| | | CTGGCAAG |
| 120 | TPP-47648 | GACATTCAGATGACACAAAGCCCATCCAGCCTTAGCGCAT |
| | CD20 | CTGTAGGAGATAGAGTTACTATAACTTGTCGGGCCTCATC |
| | Light Chain 1 | TAGTGTCAGTTACATGCACTGGTACCAGCAAAAACCCGGC |
| | | AAAGCACCCAAGCCACTTATCTATGCTCCCAGTAACCTTG |
| | | CAAGCGGCGTACCCAGCCGATTTAGTGGAAGTGGAAGCG |
| | | GAACCGACTTCACACTTACCATTTCCTCACTTCAGCCCGA |
| | | GGATTTCGCCACCTACTATTGCCAGCAGTGGAGTTTTAAC |
| | | CCTCCCACTTTTGGACAAGGAACAAAAGTTGAAATAAAAG |
| | | GCCAACCGAAGGCCGCTCCTTCCGTGAGGCTGTTCCCTCC |
| | | ATGCTCCGAGGAACTGCAGGCCAACAAGGCTACCCTCGTG |
| | | TGCCTGATCTCCGACTTTTACCCTGGCGCTGTGACCGTGGC |
| | | CTGGAAGGCTGATAGTTCTCCTGTGAAGGCCGGCGTGGAA |
| | | ACCACCACACCTTCCAAGCAGTCCAACAACAAATACGCCG |
| | | CCTCCTCCTACCTGTCTCTGACCCCTGAACAGTGGAAGTCC |
| | | CACCGGTCCTACAGCTGCCAAGTGACCCATGAGGGCTCCA |
| | | CCGTGGAAAAGACCGTGGCTCCTACAGAGGTGTCC |
| 121 | TPP-47648 | GAGATTGTCCTGACACAGTCCCCTGGCACCCTTAGCCTGT |
| | BCMA | CCCCCGGTGAAAGAGCGACCCTGTCCTGCCGTGCCTCACA |
| | Light Chain 2 | ATACATCTCTTCCAACAATCTGGCGTGGTACCAACAGAAG |
| | | CCCGGGCAAGCTCCCCGCCTCCTGATTTACGGGGCATCCA |
| | | ATAGAGCCACCGGGATCCCTGATAGATTCTCCGGCAGCGG |
| | | TAGCGGTACCGATTTCACCCTGACCATCTCTCGCCTGGAG |
| | | CCTGAAGACTTCGCGGTGTACTATTGTCAACAGTATGCTG |
| | | ATTCCCCAATCACATTCGGCCAGGGTACTAAGCTTGAAAT |
| | | CAAGCGGACAGTGGCCGCACCGTCCGTGTTCATCTTCCCA |
| | | CCTTCCGACGAGCAGCTGAAGTCTGGCACAGCCTCTGTCG |
| | | AGTGCCTGCTGAACAACTTCTACCCTCGGGAAGCCAAGGT |
| | | GCAGTGGAAGGTGGACAATGCCCTGCAGTCCGGCAACTCC |
| | | CAAGAGTCTGTGACCGAGCAGGACTCCAAGGACAGCACC |
| | | TACAGCCTGTCCTCCACACTGACCCTGTCCAAGGCCGACT |
| | | ACGAGAAGCACAAGGTGTACGCCTGCGAAGTGACCCATC |
| | | AGGGCCTGTCTAGCCCTGTGACCAAGTCTTTCAACCGGGG |
| | | CGAGTGC |
| 122 | TPP-47651- | GAGGTGCAGCTGGTGGAAAGTGGAGGTGGCCTGGTTAAG |
| | BCMA | CCTGGCGGGAGCCTGCGCCTGAGCTGCGCTGCCAGCGGCT |
| | Heavy Chain | TCACTTTCTCATCTTACAGCATGAACTGGGTGCGCCAGGC |
| | Knob (CD8) | CCCTGGCAAAGGCCTTGAGTGGGTTAGTAGCATCAGCGGC |
| | | CAGTCAAACTACATTTATTACGCCGATTCCGTCAAGGGCA |
| | | GGTTTACAATCTCCCGTGATAATGCGAAGAATAGCCTCTA |
| | | TCTCCAGATGAATTCCCTGCGCGCCGAAGATACAGCCGTG |
| | | TATTACTGCGCGAGGGGCGGGAATTACTTTGTTGAATACT |
| | | TCCAGTACTGGGGCCAGGGTACCCTCGTCACCGTTTCCAG |
| | | TGCAAGTACAAAGGGACCTTCTGTGTGTCCTCTGGCTCCC |
| | | TCCAGCAAGTCTACCTCTGGTGGAACAGCTGACCTGGGCT |
| | | GCCTGGTCAAGGATTACTTTCCTGAGCCTGTGACCGTGTC |
| | | CTGGAACTCTGGCGCTCTGACATCTGGCGTGCACACCTTT |
| | | CCAGCTGTGCTGCAGTCCTCCGGCCTGTACTCTCTGTCCTC |
| | | TGTCGTGACCGTGCCTTCCAGCTCTCTGGGCACCCAGACC |
| | | TACATCTGCAATGTGAACCACAAGCCTTCCAACACCAAGG |
| | | TGGACAAGAGAGTGGAACCAAAAAGCGTGGACAAGACCG |
| | | GAGGATCTGAGGTACAGCTGCTGGAATCAGGAGGCGGCT |
| | | TGGTTCAGCCAGGAGGCTCACTTCGGCTCTCCTGCGCAGC |
| | | AAGTGGGTTTACATTTACTGATTATGCTATAGGCTGGTTTC |
| | | GGCAAGCACCTGGTAAGGAACGCGAAGGTGTGTCATGTA |
| | | TTCGCGTAAGCGACGGATCAACATACTATGCAGACAGTGT |
| | | CAAAGGTAGATTTACTATTAGTCGTGACAGCTCAAAGAAT |
| | | ACTCTGTACTTGCAAATGAATTCCCTTCGTGCCGAAGATA |
| | | CTGCCGTTTACTATTGCGCCGCAGGATCACTCTACACCTGC |
| | | GTGCAGAGCATTGTTTGGCCTGCCAGGCCTTATTATGATA |
| | | TGGACTATTGGGGCCAAGGCACTTTGGTCACTGTTAGCTC |
| | | TGGTGGCGGAGGATCTACCCACACCTGTCCTCCATGCCCT |
| | | GCTCCACCCGTGGCCGGCCCATCCGTGTTTCTGTTCCCTCC |
| | | AAAGCCTAAGGACACCCTGATGATCTCTCGGACCCCTGAA |
| | | GTGACCTGCGTGGTGGTGGATGTGAAGCACGAGGATCCCG |
| | | AAGTGAAGTTCAATTGGTACGTGGACGGCGTGGAAGTGC |
| | | ACAACGCCAAGACCAAGCCTAGAGAGGAACAGTACAACT |
| | | CCACCTACAGAGTGGTGTCCGTGCTGACCGTGCTGCACCA |
| | | GGATTGGCTGAACGGCAAAGAGTACAAGTGCAAGGTGTC |
| | | CAACAAGGCCCTGCCTGCCCCCATCGAAAAGACCATCTCC |
| | | AAGGCCAAGGGCCAGCCTAGGGAACCCCAGGTTTACACC |
| | | CTGCCACCTTGCCGGGAAGAGATGACCAAGAACCAGGTG |
| | | TCCCTGTGGTGCCTCGTGAAGGGCTTCTACCCTTCCGATAT |
| | | CGCCGTGGAATGGGAGAGCAATGGCCAGCCTGAGAACAA |
| | | CTACAAGACAACCCCTCCTGTGCTGGACTCCGACGGCTCA |
| | | TTCTTCCTGTACTCCAAGCTGACAGTGGACAAGTCCAGAT |
| | | GGCAGCAGGGCAACGTGTTCTCCTGCTCCGTGATGCACGA |
| | | GGCCCTGCACAATCACTACACCCAGAAGTCCCTGTCTCTG |
| | | TCCCCTGGAAAA |
| 123 | TPP-47651- | GAGGTGCAGCTGGTGGAATCCGGTGGGGGTCTGGTACAG |
| | CD20 | CCTGGGGGTTCACTGAGATTGTCATGCGCAGCCTCCGGGT |
| | Heavy Chain | ACACATTCACATCATACAATATGCACTGGGTGAGGCAAGC |
| | Hole (TCR) | TCCTGGCAAAGGGTTGGAATGGGTAGGAGCAATTTATCCT |
| | | GGCAGCGGCGACACCTCATATAACCAAAAATTTAAAGGTC |
| | | GGTTTACCATAAGTGTCGACAAATCCAAAAACACCTTGTA |
| | | TTTGCAGATGAACTCACTGCGAGCTGAAGACACAGCAGTC |
| | | TATTACTGTGCTCGTGTAGTATATTACTCAAATTCCTACTG |
| | | GTATTTTGATGTATGGGGGCAGGGCACTCTCGTGACTGTA |
| | | TCAAGCGCAAGTACAAAGGGACCTTCTGTGTTCCCTCTGG |
| | | CTCCCTCCAGCAAGTCTACATCTGGTGGAACCGCTGCTCT |
| | | GGGCTGCCTGGTCAAGGATTACTTTCCTGAGCCTGTGACC |
| | | GTGTCCTGGAATTCTGGTGCTCTGACCTCCGGCGTGCACA |
| | | CCTTTCCAGCTGTGCTGCAGTCCTCTGGCCTGTACTCTCTG |
| | | AAGTCTGTCGTGACCGTGCCTTCTAGCTCTCTGGGCACCC |
| | | AGACCTACATCTGCAACGTGAACCACAAGCCTTCCAACAC |
| | | CAAGGTGGACAAGAGAGTGGAACCAAAAAGCTGCGACAA |
| | | GACCGGAGGATCTGAAGTGCAGCTGGTCGAGTCTGGCGG |
| | | AGGATTGGTTCAACCTGGCGGCTCCCTGAGACTGTCTTGT |
| | | GTGGCTAGCGGAGATGTGCATAAGATCAATTTTCTCGGCT |
| | | GGTACAGACAGGCCCCTGGCAAAGAGAGAGAGAAGGTCG |
| | | CCCACATCTCCATCGGCGACCAGACCGATTACGCCGACTC |
| | | TGCCAAGGGCAGATTCACCATCTCTCGGGACGAGTCCAAG |
| | | AACATGGTGTACCTGCAGATGAACTCCCTGAAGCCTGAGG |
| | | ATACCGCCGTGTACTTCTGCCGGGCCTTCTCTCGGATCTAC |
| | | CCCTACGATTATTGGGGCCAGGGCACCCTGGTCACAGTGT |
| | | CATCTGGTGGCGGAGGATCTACCCACACCTGTCCTCCATG |
| | | CCCTGCTCCACCCGTGGCCGGCCCTTCCGTGTTCCTGTTTC |
| | | CTCCAAAGCCTAAGGACACCCTGATGATCTCTCGGACCCC |
| | | TGAAGTGACCTGCGTGGTGGTGGATGTGAAGCACGAGGA |
| | | TCCCGAAGTGAAGTTCAATTGGTACGTGGACGGCGTGGAA |
| | | GTGCACAACGCCAAGACCAAGCCTAGAGAGGAACAGTAC |
| | | AACTCCACCTACAGAGTGGTGTCCGTGCTGACCGTGCTGC |
| | | ACCAGGATTGGCTGAACGGCAAAGAGTACAAGTGCAAGG |
| | | TGTCCAACAAGGCCCTGCCTGCCCCCATCGAAAAGACCAT |
| | | CTCTAAGGCTAAGGGCCAGCCTCGCGAGCCTCAAGTCTGT |
| | | ACACTGCCTCCTAGCCGGGAAGAGATGACCAAGAACCAG |
| | | GTGTCCCTGTCTTGCGCCGTGAAGGGCTTCTACCCTTCCGA |
| | | TATCGCCGTGGAATGGGAGTCCAATGGCCAGCCTGAGAAC |
| | | AACTACAAGACAACCCCTCCTGTGCTGGACTCCGACGGCT |
| | | CATTCTTCCTGGTGTCCAAGCTGACAGTGGACAAGTCCAG |
| | | ATGGCAGCAGGGCAACGTGTTCTCCTGCTCTGTGATGCAC |
| | | GAGGCCCTGCACAACCGGTTCACCCAGAAATCCCTGTCTC |
| | | TGTCCCCTGGCAAG |
| 124 | TPP-47651- | GAGATTGTCCTGACACAGTCCCCTGGCACCCTTAGCCTGT |
| | BCMA | CCCCCGGTGAAAGAGCGACCCTGTCCTGCCGTGCCTCACA |
| | Light Chain 1 | ATACATCTCTTCCAACAATCTGGCGTGGTACCAACAGAAG |
| | | CCCGGGCAAGCTCCCCGCCTCCTGATTTACGGGGCATCCA |
| | | ATAGAGCCACCGGGATCCCTGATAGATTCTCCGGCAGCGG |
| | | TAGCGGTACCGATTTCACCCTGACCATCTCTCGCCTGGAG |
| | | CCTGAAGACTTCGCGGTGTACTATTGTCAACAGTATGCTG |
| | | ATTCCCCAATCACATTCGGCCAGGGTACTAAGCTTGAAAT |
| | | CAAGGGCCAACCGAAGGCCGCTCCTTCCGTGAGGCTGTTC |
| | | CCTCCATGCTCCGAGGAACTGCAGGCCAACAAGGCTACCC |
| | | TCGTGTGCCTGATCTCCGACTTTTACCCTGGCGCTGTGACC |
| | | GTGGCCTGGAAGGCTGATAGTTCTCCTGTGAAGGCCGGCG |
| | | TGGAAACCACCACACCTTCCAAGCAGTCCAACAACAAATA |
| | | CGCCGCCTCCTCCTACCTGTCTCTGACCCCTGAACAGTGG |
| | | AAGTCCCACCGGTCCTACAGCTGCCAAGTGACCCATGAGG |
| | | GCTCCACCGTGGAAAAGACCGTGGCTCCTACAGAGGTGTC |
| | | C |
| 125 | TPP-47651- | GACATTCAGATGACACAAAGCCCATCCAGCCTTAGCGCAT |
| | CD20 | CTGTAGGAGATAGAGTTACTATAACTTGTCGGGCCTCATC |
| | Light Chain 2 | TAGTGTCAGTTACATGCACTGGTACCAGCAAAAACCCGGC |
| | | AAAGCACCCAAGCCACTTATCTATGCTCCCAGTAACCTTG |
| | | CAAGCGGCGTACCCAGCCGATTTAGTGGAAGTGGAAGCG |
| | | GAACCGACTTCACACTTACCATTTCCTCACTTCAGCCCGA |
| | | GGATTTCGCCACCTACTATTGCCAGCAGTGGAGTTTTAAC |
| | | CCTCCCACTTTTGGACAAGGAACAAAAGTTGAAATAAAAC |
| | | GGACAGTGGCCGCACCGTCCGTGTTCATCTTCCCACCTTCC |
| | | GACGAGCAGCTGAAGTCTGGCACAGCCTCTGTCGAGTGCC |
| | | TGCTGAACAACTTCTACCCTCGGGAAGCCAAGGTGCAGTG |
| | | GAAGGTGGACAATGCCCTGCAGTCCGGCAACTCCCAAGA |
| | | GTCTGTGACCGAGCAGGACTCCAAGGACAGCACCTACAG |
| | | CCTGTCCTCCACACTGACCCTGTCCAAGGCCGACTACGAG |
| | | AAGCACAAGGTGTACGCCTGCGAAGTGACCCATCAGGGC |
| | | CTGTCTAGCCCTGTGACCAAGTCTTTCAACCGGGGCGAGT |
| | | GC |
| 126 | TPP-47645- | GAGGTGCAGCTGGTGGAATCCGGTGGGGGTCTGGTACAG |
| | knob (CD20- | CCTGGGGGTTCACTGAGATTGTCATGCGCAGCCTCCGGGT |
| | CD8) | ACACATTCACATCATACAATATGCACTGGGTGAGGCAAGC |
| | | TCCTGGCAAAGGGTTGGAATGGGTAGGAGCAATTTATCCT |
| | | GGCAGCGGCGACACCTCATATAACCAAAAATTTAAAGGTC |
| | | GGTTTACCATAAGTGTCGACAAATCCAAAAACACCTTGTA |
| | | TTTGCAGATGAACTCACTGCGAGCTGAAGACACAGCAGTC |
| | | TATTACTGTGCTCGTGTAGTATATTACTCAAATTCCTACTG |
| | | GTATTTTGATGTATGGGGGCAGGGCACTCTCGTGACTGTA |
| | | TCAAGCGCAAGCACCAAGGGACCCTCTGTGTTCCCTCTGG |
| | | CTCCTTCCAGCAAGTCTACCTCTGGCGGAACAGCTGCTCT |
| | | GGGCTGCCTGGTCAAGGACTACTTTCCTGAGCCTGTGACC |
| | | GTGTCCTGGAACTCTGGCGCTCTGACATCTGGCGTGCACA |
| | | CCTTTCCAGCTGTGCTGCAGTCCTCCGGCCTGTACTCTCTG |
| | | TCCTCTGTCGTGACCGTGCCTTCCAGCTCTCTGGGAACCCA |
| | | GACCTACATCTGCAATGTGAACCACAAGCCTTCCAACACC |
| | | AAGGTGGACAAGAGAGTGGAACCCAAGTCCTGCGATAAG |
| | | ACCGGCGGATCTGAGGTACAGCTGCTGGAATCAGGAGGC |
| | | GGCTTGGTTCAGCCAGGAGGCTCACTTCGGCTCTCCTGCG |
| | | CAGCAAGTGGGTTTACATTTACTGATTATGCTATAGGCTG |
| | | GTTTCGGCAAGCACCTGGTAAGGAACGCGAAGGTGTGTCA |
| | | TGTATTCGCGTAAGCGACGGATCAACATACTATGCAGACA |
| | | GTGTCAAAGGTAGATTTACTATTAGTCGTGACAGCTCAAA |
| | | GAATACTCTGTACTTGCAAATGAATTCCCTTCGTGCCGAA |
| | | GATACTGCCGTTTACTATTGCGCCGCAGGATCACTCTACA |
| | | CCTGCGTGCAGAGCATTGTTTGGCCTGCCAGGCCTTATTAT |
| | | GATATGGACTATTGGGGCCAAGGCACTTTGGTCACTGTTA |
| | | GCTCTGGAGGCGGAGGCAGCACCCATACCTGTCCTCCATG |
| | | TCCTGCTCCTCCAGTGGCTGGCCCTTCCGTGTTTCTGTTCC |
| | | CTCCAAAGCCTAAGGACACCCTGATGATCTCTCGGACCCC |
| | | TGAAGTGACCTGCGTGGTGGTGGATGTGAAGCACGAGGA |
| | | TCCCGAAGTGAAGTTCAATTGGTACGTGGACGGCGTGGAA |
| | | GTGCACAACGCCAAGACCAAGCCTAGAGAGGAACAGTAC |
| | | AACTCCACCTACAGAGTGGTGTCCGTGCTGACCGTGCTGC |
| | | ACCAGGATTGGCTGAACGGCAAAGAGTACAAGTGCAAGG |
| | | TGTCCAACAAGGCCCTGCCTGCTCCTATCGAAAAGACCAT |
| | | CTCCAAGGCCAAGGGACAGCCCAGGGAACCCCAGGTTTA |
| | | CACCCTGCCTCCATGCCGGGAAGAGATGACCAAGAACCA |
| | | GGTGTCCCTGTGGTGCCTGGTTAAGGGCTTCTACCCCTCCG |
| | | ATATCGCCGTGGAATGGGAGTCTAATGGCCAGCCTGAGAA |
| | | CAACTACAAGACCACACCTCCTGTGCTGGACTCCGACGGC |
| | | TCATTCTTCCTGTACTCCAAGCTGACAGTGGACAAGTCCA |
| | | GATGGCAGCAGGGCAACGTGTTCTCCTGCTCCGTGATGCA |
| | | CGAGGCCCTGCACAATCACTACACCCAGAAGTCCCTGTCT |
| | | CTGAGCCCCGGCAAA |
| 127 | TPP-47645 | GAGGTGCAGCTGGTGGAATCCGGTGGGGGTCTGGTACAG |
| | hole (CD20- | CCTGGGGGTTCACTGAGATTGTCATGCGCAGCCTCCGGGT |
| | TCR) | ACACATTCACATCATACAATATGCACTGGGTGAGGCAAGC |
| | | TCCTGGCAAAGGGTTGGAATGGGTAGGAGCAATTTATCCT |
| | | GGCAGCGGCGACACCTCATATAACCAAAAATTTAAAGGTC |
| | | GGTTTACCATAAGTGTCGACAAATCCAAAAACACCTTGTA |
| | | TTTGCAGATGAACTCACTGCGAGCTGAAGACACAGCAGTC |
| | | TATTACTGTGCTCGTGTAGTATATTACTCAAATTCCTACTG |
| | | GTATTTTGATGTATGGGGGCAGGGCACTCTCGTGACTGTA |
| | | TCAAGCGCAAGTACTAAGGGCCCCTCTGTGTTCCCTCTGG |
| | | CTCCTTCCAGCAAGTCTACCTCTGGCGGAACAGCTGCTCT |
| | | GGGCTGCCTGGTCAAGGACTACTTTCCTGAGCCTGTGACC |
| | | GTGTCCTGGAACTCTGGCGCTCTGACATCTGGCGTGCACA |
| | | CCTTTCCAGCTGTGCTGCAGTCCTCCGGCCTGTACTCTCTG |
| | | TCCTCTGTCGTGACCGTGCCTTCCAGCTCTCTGGGAACCCA |
| | | GACCTACATCTGCAATGTGAACCACAAGCCTTCCAACACC |
| | | AAGGTGGACAAGAGAGTGGAACCCAAGTCCTGCGATAAG |
| | | ACCGGCGGATCTGAGGTGCAGCTGGTGGAATCTGGCGGA |
| | | GGATTGGTTCAGCCTGGCGGCTCTCTGAGACTGTCTTGTGT |
| | | GGCTAGCGGCGACGTGCACAAGATCAACTTTCTCGGCTGG |
| | | TACAGACAGGCCCCTGGCAAAGAGAGAGAGAAGGTCGCC |
| | | CACATCTCCATCGGCGACCAGACCGATTACGCCGACTCTG |
| | | CCAAGGGCAGATTCACCATCTCTCGGGACGAGTCCAAGAA |
| | | CATGGTGTACCTGCAGATGAACTCCCTGAAGCCTGAGGAT |
| | | ACCGCCGTGTACTTCTGCCGGGCCTTCTCTCGGATCTACCC |
| | | CTACGATTATTGGGGCCAGGGCACCCTGGTCACAGTTTCT |
| | | AGCGGCGGAGGCGGCTCTACCCATACCTGTCCTCCATGTC |
| | | CTGCTCCTCCAGTGGCTGGCCCATCCGTGTTTCTGTTCCCT |
| | | CCAAAGCCTAAGGACACCCTGATGATCTCTCGGACCCCTG |
| | | AAGTGACCTGCGTGGTGGTGGATGTGAAGCACGAGGATC |
| | | CCGAAGTGAAGTTCAATTGGTACGTGGACGGCGTGGAAGT |
| | | GCACAACGCCAAGACCAAGCCTAGAGAGGAACAGTACAA |
| | | CTCCACCTACAGAGTGGTGTCCGTGCTGACCGTGCTGCAC |
| | | CAGGATTGGCTGAACGGCAAAGAGTACAAGTGCAAGGTG |
| | | TCCAACAAGGCCCTGCCTGCTCCTATCGAAAAGACCATCT |
| | | CCAAGGCCAAGGGCCAGCCTCGGGAACCTCAAGTCTGTAC |
| | | CCTGCCTCCTAGCCGGGAAGAGATGACCAAGAACCAGGT |
| | | GTCCCTGTCCTGTGCCGTGAAGGGCTTCTACCCTTCCGATA |
| | | TCGCCGTGGAATGGGAGAGCAATGGCCAGCCTGAGAACA |
| | | ACTACAAGACCACACCTCCTGTGCTGGACTCCGACGGCTC |
| | | ATTCTTCCTGGTGTCCAAGCTGACAGTGGACAAGTCCAGA |
| | | TGGCAGCAGGGCAACGTGTTCTCCTGCTCCGTGATGCACG |
| | | AGGCCCTGCACAACAGGTTCACCCAGAAGTCCCTGTCTCT |
| | | GAGCCCTGGCAAG |
| 128 | TPP-47645 | GACATTCAGATGACACAAAGCCCATCCAGCCTTAGCGCAT |
| | (light chain) | CTGTAGGAGATAGAGTTACTATAACTTGTCGGGCCTCATC |
| | | TAGTGTCAGTTACATGCACTGGTACCAGCAAAAACCCGGC |
| | | AAAGCACCCAAGCCACTTATCTATGCTCCCAGTAACCTTG |
| | | CAAGCGGCGTACCCAGCCGATTTAGTGGAAGTGGAAGCG |
| | | GAACCGACTTCACACTTACCATTTCCTCACTTCAGCCCGA |
| | | GGATTTCGCCACCTACTATTGCCAGCAGTGGAGTTTTAAC |
| | | CCTCCCACTTTTGGACAAGGAACAAAAGTTGAAATAAAAC |
| | | GGACAGTGGCCGCACCGTCCGTGTTCATCTTCCCACCTTCC |
| | | GACGAGCAGCTGAAGTCTGGCACAGCCTCTGTCGTGTGCC |
| | | TGCTGAACAACTTCTACCCTCGGGAAGCCAAGGTGCAGTG |
| | | GAAGGTGGACAATGCCCTGCAGTCCGGCAACTCCCAAGA |
| | | GTCTGTGACCGAGCAGGACTCCAAGGACAGCACCTACAG |
| | | CCTGTCCTCCACACTGACCCTGTCCAAGGCCGACTACGAG |
| | | AAGCACAAGGTGTACGCCTGCGAAGTGACCCATCAGGGC |
| | | CTGTCTAGCCCTGTGACCAAGTCTTTCAACCGGGGCGAGT |
| | | GC |
| 129 | TPP-47646- | GAGGTGCAGCTGGTGGAAAGTGGAGGTGGCCTGGTTAAG |
| | knob | CCTGGCGGGAGCCTGCGCCTGAGCTGCGCTGCCAGCGGCT |
| | (BCMA-CD8) | TCACTTTCTCATCTTACAGCATGAACTGGGTGCGCCAGGC |
| | | CCCTGGCAAAGGCCTTGAGTGGGTTAGTAGCATCAGCGGC |
| | | CAGTCAAACTACATTTATTACGCCGATTCCGTCAAGGGCA |
| | | GGTTTACAATCTCCCGTGATAATGCGAAGAATAGCCTCTA |
| | | TCTCCAGATGAATTCCCTGCGCGCCGAAGATACAGCCGTG |
| | | TATTACTGCGCGAGGGGGGGGAATTACTTTGTTGAATACT |
| | | TCCAGTACTGGGGCCAGGGTACCCTCGTCACCGTTTCCAG |
| | | TGCAAGCACCAAGGGACCCTCTGTGTTCCCTCTGGCTCCTT |
| | | CCAGCAAGTCTACCTCTGGCGGAACAGCTGCTCTGGGCTG |
| | | CCTGGTCAAGGACTACTTTCCTGAGCCTGTGACCGTGTCCT |
| | | GGAACTCTGGCGCTCTGACATCTGGCGTGCACACCTTTCC |
| | | AGCTGTGCTGCAGTCCTCCGGCCTGTACTCTCTGTCCTCTG |
| | | TCGTGACCGTGCCTTCCAGCTCTCTGGGAACCCAGACCTA |
| | | CATCTGCAATGTGAACCACAAGCCTTCCAACACCAAGGTG |
| | | GACAAGAGAGTGGAACCCAAGTCCTGCGATAAGACCGGC |
| | | GGATCTGAGGTACAGCTGCTGGAATCAGGAGGCGGCTTG |
| | | GTTCAGCCAGGAGGCTCACTTCGGCTCTCCTGCGCAGCAA |
| | | GTGGGTTTACATTTACTGATTATGCTATAGGCTGGTTTCGG |
| | | CAAGCACCTGGTAAGGAACGCGAAGGTGTGTCATGTATTC |
| | | GCGTAAGCGACGGATCAACATACTATGCAGACAGTGTCA |
| | | AAGGTAGATTTACTATTAGTCGTGACAGCTCAAAGAATAC |
| | | TCTGTACTTGCAAATGAATTCCCTTCGTGCCGAAGATACT |
| | | GCCGTTTACTATTGCGCCGCAGGATCACTCTACACCTGCG |
| | | TGCAGAGCATTGTTTGGCCTGCCAGGCCTTATTATGATAT |
| | | GGACTATTGGGGCCAAGGCACTTTGGTCACTGTTAGCTCT |
| | | GGAGGCGGAGGCAGCACCCATACCTGTCCTCCATGTCCTG |
| | | CTCCTCCAGTGGCTGGCCCTTCCGTGTTTCTGTTCCCTCCA |
| | | AAGCCTAAGGACACCCTGATGATCTCTCGGACCCCTGAAG |
| | | TGACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCTCCTATCGAAAAGACCATCTCCAA |
| | | GGCCAAGGGACAGCCCAGGGAACCCCAGGTTTACACCCT |
| | | GCCTCCATGCCGGGAAGAGATGACCAAGAACCAGGTGTC |
| | | CCTGTGGTGCCTGGTTAAGGGCTTCTACCCCTCCGATATCG |
| | | CCGTGGAATGGGAGTCTAATGGCCAGCCTGAGAACAACT |
| | | ACAAGACCACACCTCCTGTGCTGGACTCCGACGGCTCATT |
| | | CTTCCTGTACTCCAAGCTGACAGTGGACAAGTCCAGATGG |
| | | CAGCAGGGCAACGTGTTCTCCTGCTCCGTGATGCACGAGG |
| | | CCCTGCACAATCACTACACCCAGAAGTCCCTGTCTCTGAG |
| | | CCCCGGCAAA |
| 130 | TPP-47646 | GAGGTGCAGCTGGTGGAAAGTGGAGGTGGCCTGGTTAAG |
| | hole (BCMA- | CCTGGCGGGAGCCTGCGCCTGAGCTGCGCTGCCAGCGGCT |
| | TCR) | TCACTTTCTCATCTTACAGCATGAACTGGGTGCGCCAGGC |
| | | CCCTGGCAAAGGCCTTGAGTGGGTTAGTAGCATCAGCGGC |
| | | CAGTCAAACTACATTTATTACGCCGATTCCGTCAAGGGCA |
| | | GGTTTACAATCTCCCGTGATAATGCGAAGAATAGCCTCTA |
| | | TCTCCAGATGAATTCCCTGCGCGCCGAAGATACAGCCGTG |
| | | TATTACTGCGCGAGGGGGGGGAATTACTTTGTTGAATACT |
| | | TCCAGTACTGGGGCCAGGGTACCCTCGTCACCGTTTCCAG |
| | | TGCAAGCACCAAGGGCCCATCCGTCTTCCCCCTGGCACCC |
| | | TCCTCCAAGAGCACCTCTGGGGGCACAGCGGCCCTGGGCT |
| | | GCCTGGTCAAGGACTACTTCCCCGAACCGGTGACGGTGTC |
| | | GTGGAACTCAGGCGCCCTGACCAGCGGCGTGCACACCTTC |
| | | CCGGCTGTCCTACAGTCCTCAGGACTCTACTCCCTCAGCA |
| | | GCGTGGTGACCGTGCCCTCCAGCAGCTTGGGCACCCAGAC |
| | | CTACATCTGCAACGTGAATCACAAGCCCAGCAACACCAAG |
| | | GTGGACAAGAGAGTTGAACCAAAAAGCTGCGACAAGACC |
| | | GGAGGATCTGAAGTGCAGCTGGTCGAGTCTGGCGGAGGA |
| | | TTGGTTCAACCTGGCGGCTCCCTGAGACTGTCTTGTGTGGC |
| | | TAGCGGAGATGTGCATAAGATCAATTTTCTCGGCTGGTAC |
| | | AGACAGGCCCCTGGCAAAGAGAGAGAGAAGGTCGCCCAC |
| | | ATCTCCATCGGCGACCAGACCGATTACGCCGACTCTGCCA |
| | | AGGGCAGATTCACCATCTCTCGGGACGAGTCCAAGAACAT |
| | | GGTGTACCTGCAGATGAACTCCCTGAAGCCTGAGGATACC |
| | | GCCGTGTACTTCTGCCGGGCCTTCTCTCGGATCTACCCCTA |
| | | CGATTATTGGGGCCAGGGCACCCTGGTCACAGTGTCATCT |
| | | GGTGGCGGAGGATCTACCCACACCTGTCCTCCATGCCCTG |
| | | CTCCACCCGTGGCCGGCCCTTCCGTGTTCCTGTTTCCTCCA |
| | | AAGCCTAAGGACACCCTGATGATCTCTCGGACCCCTGAAG |
| | | TGACCTGCGTGGTGGTGGATGTGAAGCACGAGGATCCCGA |
| | | AGTGAAGTTCAATTGGTACGTGGACGGCGTGGAAGTGCAC |
| | | AACGCCAAGACCAAGCCTAGAGAGGAACAGTACAACTCC |
| | | ACCTACAGAGTGGTGTCCGTGCTGACCGTGCTGCACCAGG |
| | | ATTGGCTGAACGGCAAAGAGTACAAGTGCAAGGTGTCCA |
| | | ACAAGGCCCTGCCTGCCCCCATCGAAAAGACCATCTCTAA |
| | | GGCTAAGGGCCAGCCTCGCGAGCCTCAAGTCTGTACACTG |
| | | CCTCCTAGCCGGGAAGAGATGACCAAGAACCAGGTGTCC |
| | | CTGTCTTGCGCCGTGAAGGGCTTCTACCCTTCCGATATCGC |
| | | CGTGGAATGGGAGTCCAATGGCCAGCCTGAGAACAACTA |
| | | CAAGACAACCCCTCCTGTGCTGGACTCCGACGGCTCATTC |
| | | TTCCTGGTGTCCAAGCTGACAGTGGACAAGTCCAGATGGC |
| | | AGCAGGGCAACGTGTTCTCCTGCTCTGTGATGCACGAGGC |
| | | CCTGCACAACCGGTTCACCCAGAAATCCCTGTCTCTGTCC |
| | | CCTGGCAAG |
| 13 | TPP-47646 | GAGATTGTCCTGACACAGTCCCCTGGCACCCTTAGCCTGT |
| | (light chain) | CCCCCGGTGAAAGAGCGACCCTGTCCTGCCGTGCCTCACA |
| | | ATACATCTCTTCCAACAATCTGGCGTGGTACCAACAGAAG |
| | | CCCGGGCAAGCTCCCCGCCTCCTGATTTACGGGGCATCCA |
| | | ATAGAGCCACCGGGATCCCTGATAGATTCTCCGGCAGCGG |
| | | TAGCGGTACCGATTTCACCCTGACCATCTCTCGCCTGGAG |
| | | CCTGAAGACTTCGCGGTGTACTATTGTCAACAGTATGCTG |
| | | ATTCCCCAATCACATTCGGCCAGGGTACTAAGCTTGAAAT |
| | | CAAGCGGACAGTGGCCGCACCGTCCGTGTTCATCTTCCCA |
| | | CCTTCCGACGAGCAGCTGAAGTCTGGCACAGCCTCTGTCG |
| | | TGTGCCTGCTGAACAACTTCTACCCTCGGGAAGCCAAGGT |
| | | GCAGTGGAAGGTGGACAATGCCCTGCAGTCCGGCAACTCC |
| | | CAAGAGTCTGTGACCGAGCAGGACTCCAAGGACAGCACC |
| | | TACAGCCTGTCCTCCACACTGACCCTGTCCAAGGCCGACT |
| | | ACGAGAAGCACAAGGTGTACGCCTGCGAAGTGACCCATC |
| | | AGGGCCTGTCTAGCCCTGTGACCAAGTCTTTCAACCGGGG |
| | | CGAGTGC |
| 132 | Chi with | ASTKGPSVFPLAPSSKSTSGGTADLGCLVKDYFPEPVTVSW |
| | A141D | NSGALTSGVHTFPAVLQSSGLYSLSSVVTVPSSSLGTQTYIC |
| | | NVNHKPSNTKVDKRVEPKSC |
| 133 | IgG1 CH2 | APELLGGPSVFLFPPKPKDTLMISRTPEVTCVVVDVSHEDPEV |
| | WT(IMGT) | KFNWYVDGVEVHNAKTKPREEQYNSTYRVVSVLTVLHQD |
| | alternate | WLNGKEYKCKVSNKALPAPIEKTISKAK |
| 134 | IgG1 CH2 Fc- | APPVAGPSVFLFPPKPKDTLMISRTPEVTCVVVDVKHEDPEV |
| | null (IMGT) | KFNWYVDGVEVHNAKTKPREEQYNSTYRVVSVLTVLHQD |
| | alternate | WLNGKEYKCKVSNKALPAPIEKTISKAK |
| 135 | Backbone | ASTKGPSVFPLAPSSKSTSGGTAALGCLVKDYFPEPVTVSWN |
| | (TCR/hole) | SGALTSGVHTFPAVLQSSGLYSLKSVVTVPSSSLGTQTYICNV |
| | | NHKPSNTKVDKRVEPKSCDKTGGSEVQLVESGGGLVQPGGS |
| | | LRLSCVASGDVHKINFLGWYRQAPGKEREKVAHISIGDQTD |
| | | YADSAKGRFTISRDESKNMVYLQMNSLKPEDTAVYFCRAFS |
| | | RIYPYDYWGQGTLVTVSSGGGGSTHTCPPCPAPPVAGPSVFL |
| | | FPPKPKDTLMISRTPEVTCVVVDVKHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSREEMTKNQVSLS |
| | | CAVKGFYPSDIAVEWESNGQPENNYKTTPPVLDSDGSFFLVS |
| | | KLTVDKSRWQQGNVFSCSVMHEALHNRFTQKSLSLSPGK |
| 136 | Backbone | ASTKGPSVCPLAPSSKSTSGGTADLGCLVKDYFPEPVTVSWN |
| | (CD8/knob) | SGALTSGVHTFPAVLQSSGLYSLSSVVTVPSSSLGTQTYICNV |
| | | NHKPSNTKVDKRVEPKSVDKTGGSEVQLLESGGGLVQPGGS |
| | | LRLSCAASGFTFTDYAIGWFRQAPGKEREGVSCIRVSDGSTY |
| | | YADSVKGRFTISRDSSKNTLYLQMNSLRAEDTAVYYCAAGS |
| | | LYTCVQSIVWPARPYYDMDYWGQGTLVTVSSGGGGSTHTC |
| | | PPCPAPPVAGPSVFLFPPKPKDTLMISRTPEVTCVVVDVKHED |
| | | PEVKFNWYVDGVEVHNAKTKPREEQYNSTYRVVSVLTVLH |
| | | QDWLNGKEYKCKVSNKALPAPIEKTISKAKGQPREPQVYTL |
| | | PPCREEMTKNQVSLWCLVKGFYPSDIAVEWESNGQPENNYK |
| | | TTPPVLDSDGSFFLYSKLTVDKSRWQQGNVFSCSVMHEALH |
| | | NHYTQKSLSLSPGK |
| 137 | TPP-47645 | EVQLVESGGGLVQPGGSLRLSCAASGYTFTSYNMHWVRQA |
| | hole (TCR) | PGKGLEWVGAIYPGSGDTSYNQKFKGRFTISVDKSKNTLYL |
| | CD20 HC | QMNSLRAEDTAVYYCARVVYYSNSYWYFDVWGQGTLVTV |
| | TCR (FC | SSASTKGPSVFPLAPSSKSTSGGTAALGCLVKDYFPEPVTVS |
| | hole) | WNSGALTSGVHTFPAVLQSSGLYSLSSVVTVPSSSLGTQTYIC |
| | | NVNHKPSNTKVDKRVEPKSCDKTGGSEVQLVESGGGLVQPG |
| | | GSLRLSCVASGDVHKINFLGWYRQAPGKEREKVAHISIGDQT |
| | | DYADSAKGRFTISRDESKNMVYLQMNSLKPEDTAVYFCRAF |
| | | SRIYPYDYWGQGTLVTVSSGGGGSTHTCPPCPAPPVAGPSVF |
| | | LFPPKPKDTLMISRTPEVTCVVVDVKHEDPEVKFNWYVDGV |
| | | EVHNAKTKPREEQYNSTYRVVSVLTVLHQDWLNGKEYKCK |
| | | VSNKALPAPIEKTISKAKGQPREPQVCTLPPSREEMTKNQVSL |
| | | SCAVKGFYPSDIAVEWESNGQPENNYKTTPPVLDSDGSFFLV |
| | | SKLTVDKSRWQQGNVFSCSVMHEALHNRFTQKSLSLSPGK |
| 138 | Comparator T | QLQLQESGPGLVKPSETLSLTCTVSGGSISSGSYFWGWIRQPP |
| | HC (BCMA) | GKGLEWIGSIYYSGITYYNPSLKSRVTISVDTSKNQFSLKLSS |
| | | VTAADTAVYYCARHDGAVAGLFDYWGQGTLVTVSSASTKG |
| | | PSVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEAAGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEK |
| | | TISKAKGQPREPQVYTLPPSQEEMTKNQVSLTCLVKGFYPSDI |
| | | AVEWESNGQPENNYKTTPPVLDSDGSFFLYSRLTVDKSRWQ |
| | | EGNVFSCSVMHEALHNHYTQKSLSLSLGK |

### Example 2: Production of T Cell Engagers (TCEs)

TCEs were generated by cloning methodologies with genes encoding CD20 and BCMA TCEs into expression vectors by restriction enzymes. Then, G22 CHO cells were seeded, 2×10⁶ viable cells/ml. After 24 hours, 400 ml aliquots of cells at 4×10⁶ viable cells/ml, were decanted into 2 L Roller bottles (VWR 734-1730). Cells were treated with 1% DMSO 1 hour pre-transfection (Sigma D2650) and then transfected with 1 μg DNA/ml of cells using PEI-MAX (PolySciences, product ref. #24765) at a PEI: DNA ratio of 5:1. Both the PEI-MAX and DNA were each diluted in 150 mM NaCl before combining to form a DNA-PEI complex and added directly to the cells.

Post-transfection, cultures were maintained at 37° C. for 4 hours, then the temperature was reduced to 34° C. The cultures were maintained at 34° C., 5% CO₂, 80% Humidity for 7 days and supplemented with feeds. Viable cell density, total cell density and cell viability were measured using a Vi-Cell XR cell counter (Beckman Coulter). Titer quantitation was performed by BLI assay using OCTET Protein A Biosensors (Sartorius, 185010). After, the protein was purified from clarified culture supernatant using an ÄKTAPure (Cytiva) and 5 ml Purolite Praesto Jetted A50 HipH resin (Ecolab Purolite). Protein was eluted from the columns using 0.2M Sodium Citrate (pH 4.7) and buffer exchanged into PBS using PD10 columns (Cytiva 17-0851-01). Protein was polished by size exclusion chromatography using a preparation grade HiLoad 26/600 column Superdex 200 pg (Cytiva 28989336). The concentration of the TCEs was determined spectrophotometrically using extinction coefficients based upon the individual amino acid sequence. Full QC was performed including SDS-PAGE and HP-SEC.

### Example 3: In Vitro Testing of TPP-47648 (BCMA:CD20) and TPP-47651 (CD20:BCMA) Binding Proteins

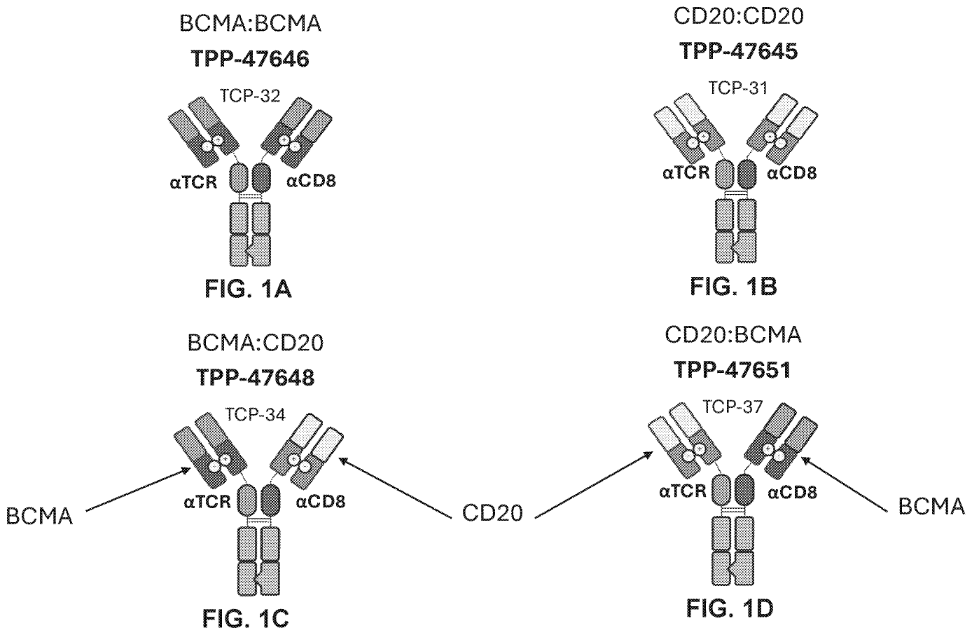

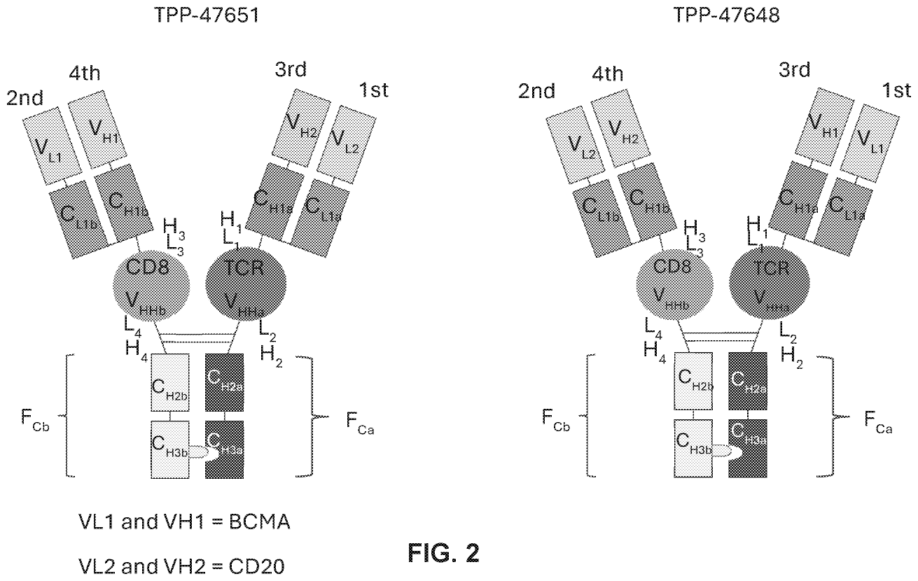

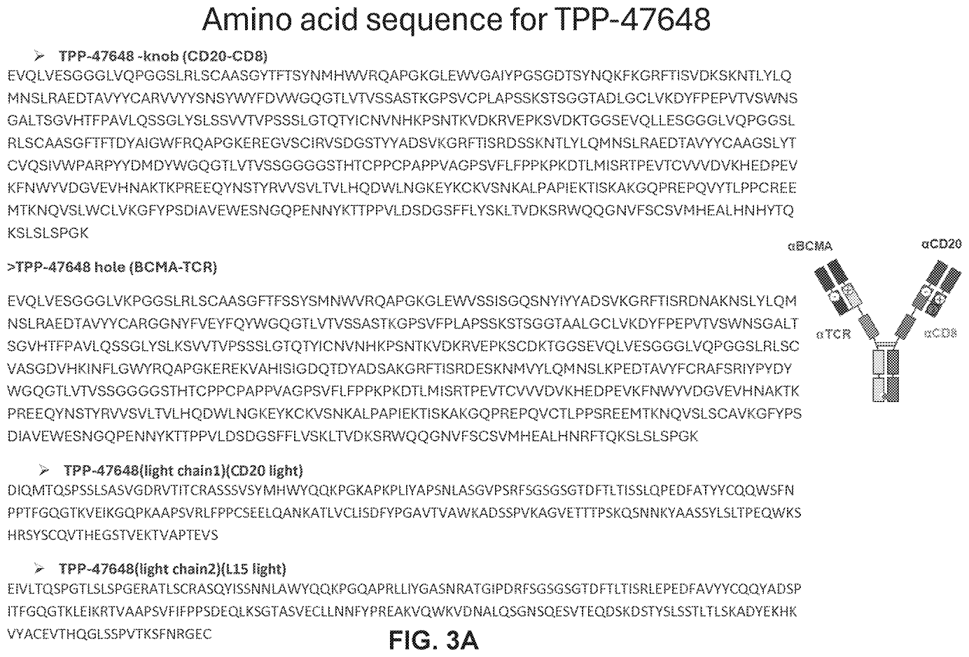

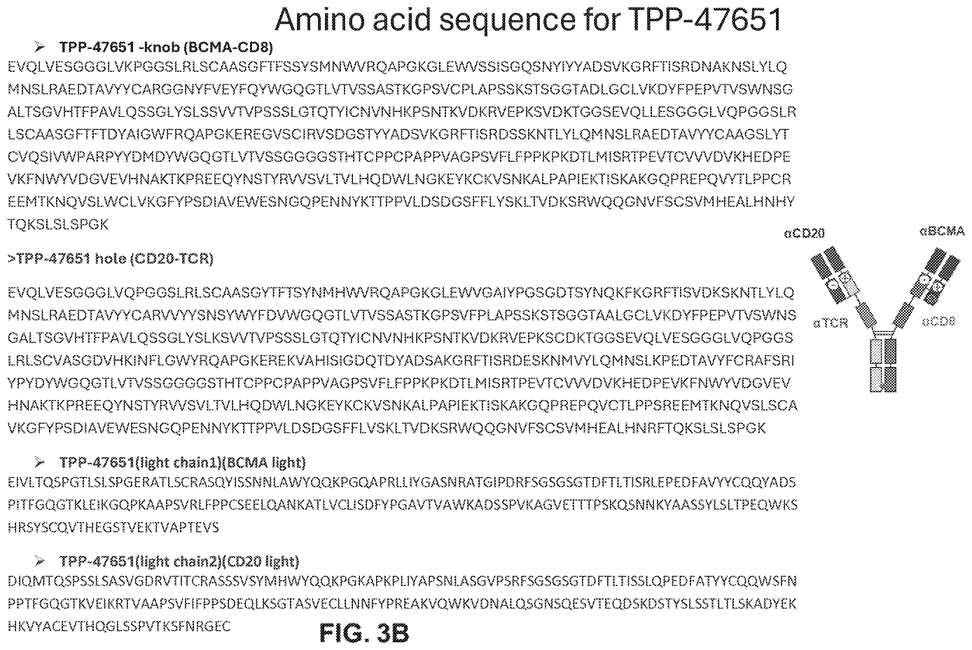

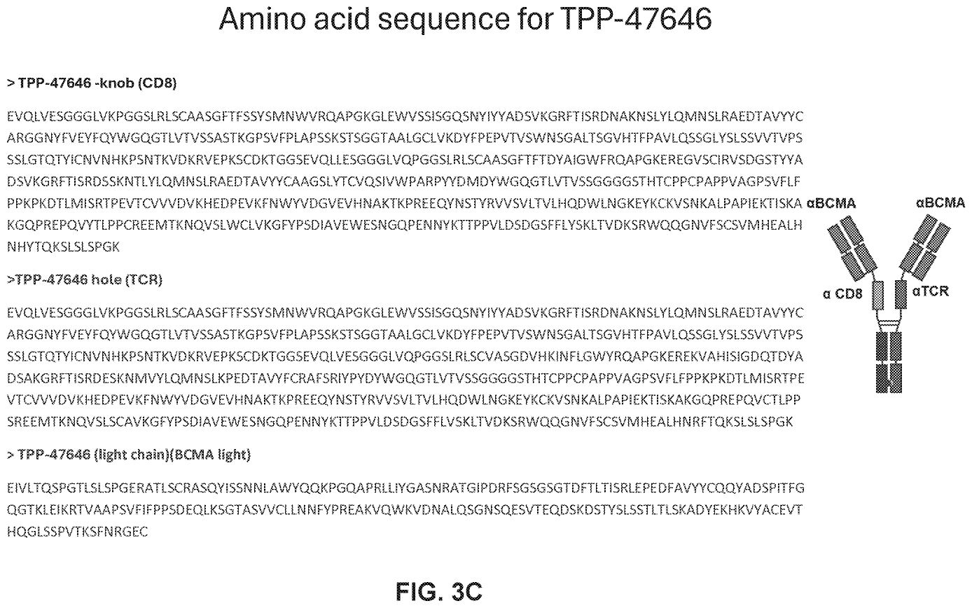

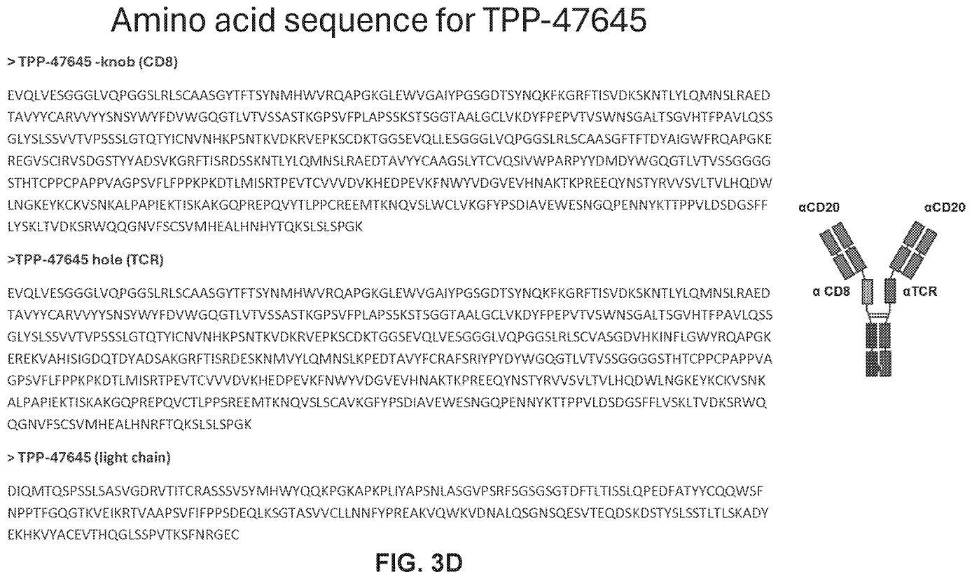

The depletion of B cells is important in the context of B cell malignancies, such as cancer, autoimmune diseases, or to prevent loss of an antibody therapeutic's efficacy through production of anti-drug antibodies (ADA). Targeting both CD20 and BCMA proteins expressed by B cells allows for B cell lineage depletion. CD20 and BCMA monospecific and bispecific TCEs were tested against B cell lines to assess cytotoxic activity and T cell activation. The activity of two TCEs each comprising a CD20 and BCMA TAA binding site (TPP-47648 FIG. 3A and FIG. 1C, TPP-47651 FIG. 1D and FIG. 3B) were compared to two TCEs each comprising either two BCMA TAA binding sites (TPP-47646, FIG. 1A and FIG. 3C) or two CD20 TAA binding sites (TPP-47645, FIG. 1B and FIG. 3D).

### In Vitro Cytolysis and Comparison of CD4 T Cell and CD8 T Cell Activation

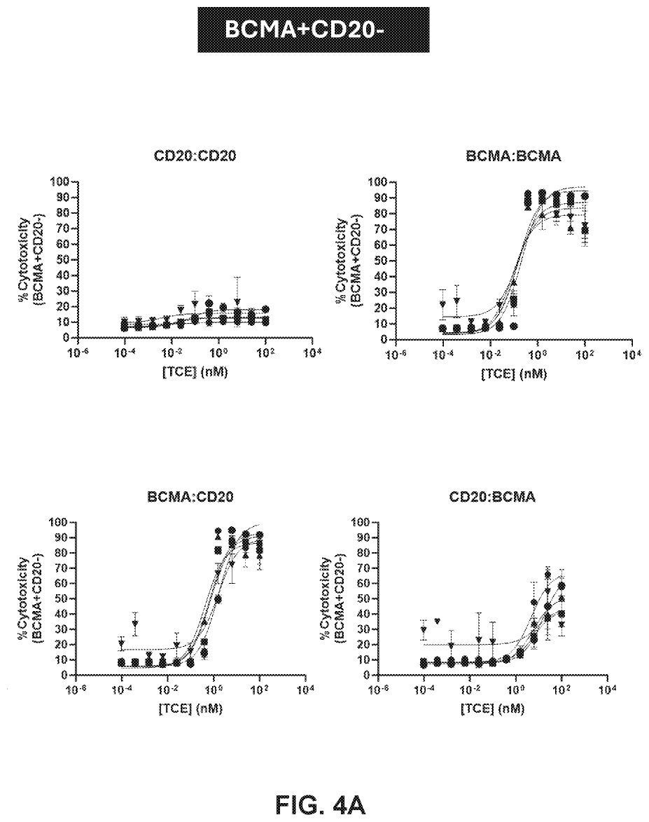

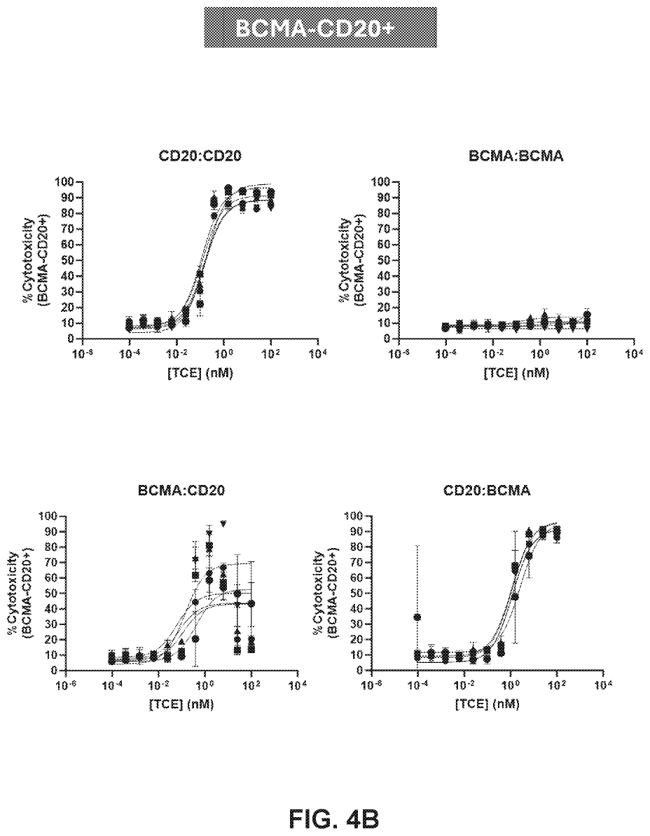

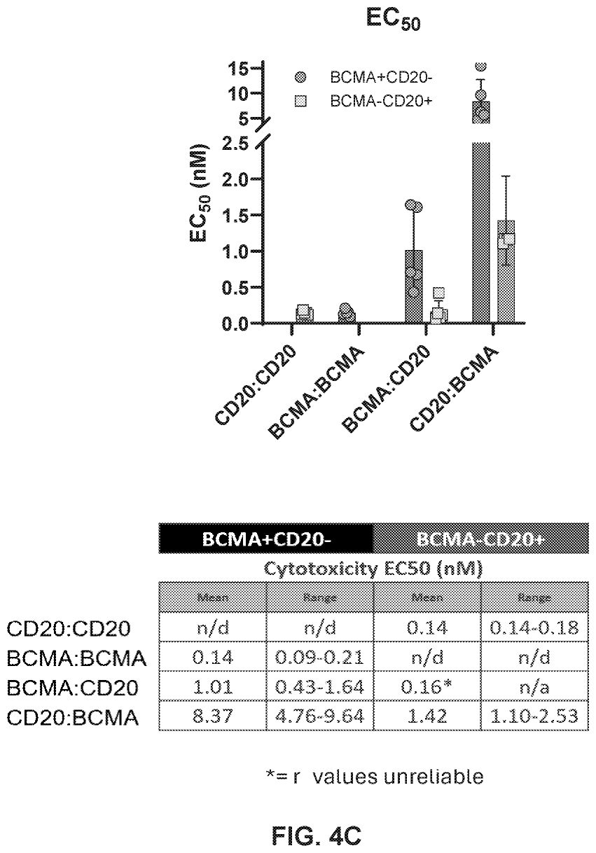

The cytolysis and activation assay was set up with purified Pan T cells cocultured with pre-labeled CellTrace violet target B cell lines (BCMA-overexpressing cell line, NCI-H929 or CD20-overexpressing cell line, REC-1) in the presence of an increasing dose of each TCE molecule (from 0.4 pM up to 100 nM) at an E:T ratio 2.5:1. Cells were incubated for 5 days at 37° C., 5% CO₂. After, cells were stained with a live/dead marker and surface antibodies to segregate T cell types and activation status. Flow cytometry was used to measure all parameters and cytotoxicity was determined by % CTV+ in dead cell gate. Correlation between cytolysis and T cells activation was assessed by measuring levels of CD25 expressing CD8 and CD4 T cells. The experiment was conducted with different donors, represented by each line on FIG. 4A and FIG. 4B. TPP-47648 and TPP-47651 demonstrated cytotoxicity against both BCMA+ and CD20+ cells with EC₅₀ₛ in the 1-10 nM range. FIG. 4C shows the corresponding EC₅₀s in the cytolysis in vitro assay.

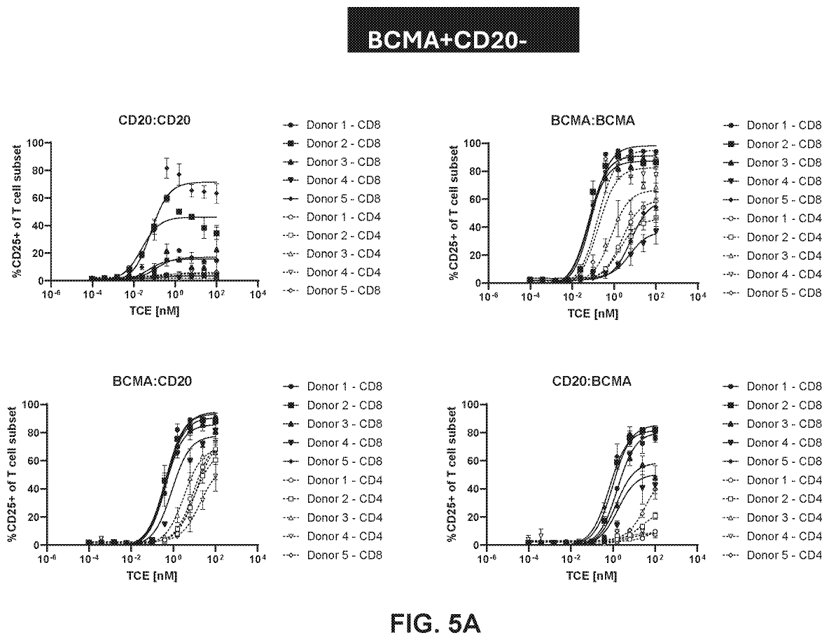

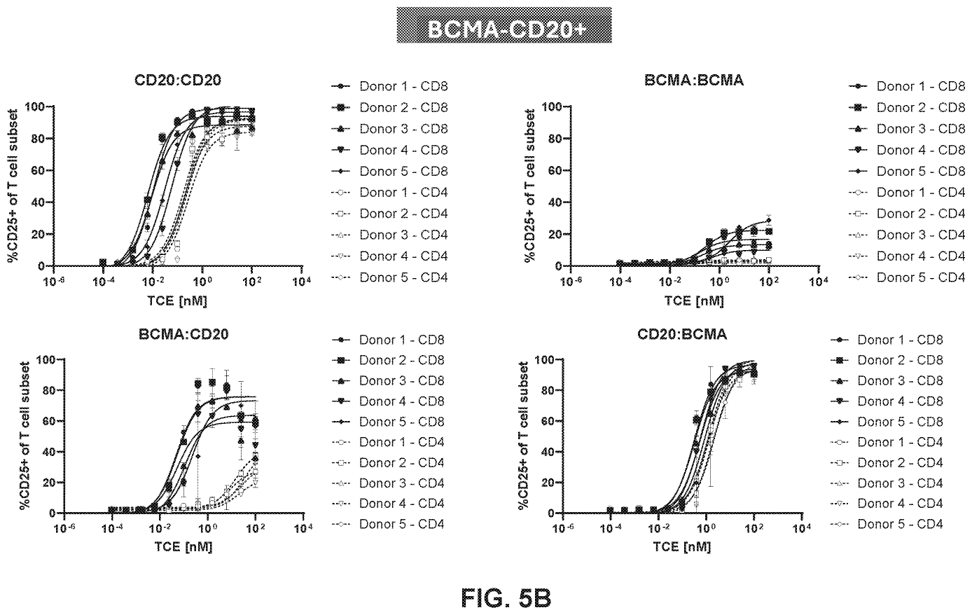

At the end of the cytolysis assay, T cell activation status was evaluated by flow cytometry. TPP-47648 and TPP-47651 both demonstrated a CD8-biased T cell activation profile, with TPP-47648 demonstrating a stronger CD8-biased T cell activation profile in the BCMA+ cell line and TPP47651 demonstrating a stronger CD8-biased T cell activation profile in the CD20+ cell line. FIG. 5A and FIG. 5B show corresponding CD4 and CD8 T cell activation profiles as % of cells expressing CD25 on the cell surface. The experiment was conducted with different donors, represented by each line on FIG. 5A and FIG. 5B.

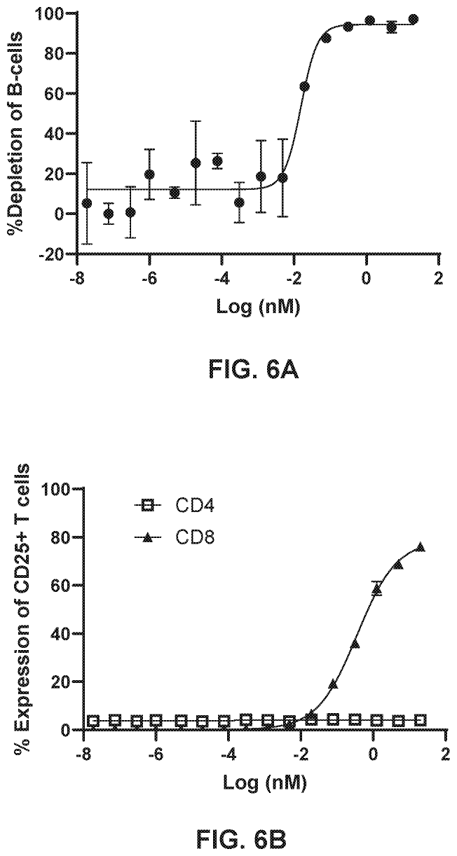

To test for in vitro T-cell effector functions on B-cells in healthy donor derived PBMCs the following experiments were performed. Healthy donor PBMCs were thawed, counted and were treated with increasing concentrations (0.00000002 nM to 20 nM) of TPP-47648 for 72 h. The level of B-cell cytolysis as well as the level of T cell activation for both CD8 and CD4 T cells were measured by flow cytometry. TPP-47648 potently induces the depletion of the B-cells in the healthy donor PBMCs with EC₅₀ of 15.5 μM (FIG. 6A). In addition, TPP-47648 induces a selective activation of CD8+ T cells and no activation of CD4+ T cells (FIG. 6B), the CD4+ T-cells being major contributors of pro-inflammatory cytokine production in autoimmune diseases.

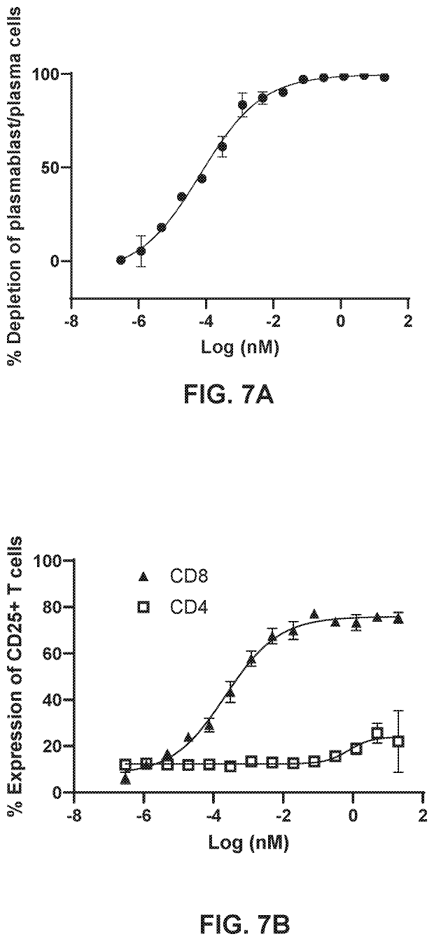

TPP-47648 was analyzed for potential targeting of plasmablasts. Plasmablasts are immature B-cell precursors that differentiate into plasma cells. Healthy donor PBMCs were thawed, counted and naïve B cells were separated by magnetic beads. The cells were cultured with 1 ug/ml CpG and 100 U/ml IL-2 for 5 days in RPMI media to induce B cell differentiation into plasmablasts. Following 5 days of differentiation, autologous T cells were isolated and co-cultured with differentiated plasmablasts at a ratio of 1:5 target to effector cells. The cells were stimulated with increasing concentrations (0.0000003 nM to 20 nM) of TPP-47648 for 72 h. The level of plasmablast cytolysis as well as the level of T cell activation for both CD8 T cells and CD4 were measured by flow cytometry. TPP-47648 potently depletes the plasmablablast/plasma cells with an EC₅₀ of 0.06 μM (FIG. 7A). In addition, TPP-47648 induces a selective activation of CD8+ T cells and minimal activation of CD4+T cells (FIG. 7B).

By combining BCMA and CD20 TAA binding sites within one T cell engager, the data above shows that it is possible to maintain sufficient cytotoxicity in BCMA positive and CD20 positive cell lines when compared to the BCMA TCE and the CD20 TCE, whilst preferentially selecting CD8 positive T cells over CD4 positive T cells which should lead to an improved safety profile. By dual targeting of B-cells (CD20) and plasma cells (BCMA), the TCEs of the disclosure should provide safer and more officious treatments for patients, as both of these target populations play a critical role in the pathogenesis and persistence of autoimmune diseases (both autoantibody-dependent and autoantibody-independent ways) and in some cancers.

### Example 4: In Vitro Testing of TPP-7648 (BCMA:CD20) in B Cells from Healthy Donor Derived PBMCs and in a Cell Line

### In Vitro Cytokine Release Profile in a BCMA+ CD20− Cell Line

In order to assess the BCMA driven depletion, a BCMA+ CD20− Cell line (NCI-H929) was co-cultured with Pan (CD3+ T) cells in the presence of increasing concentration (0.0000002 nM to 20 nM) of TPP-47648. Pan (CD3+ T) cells were isolated from frozen PBMCs and then co-cultured with NCI-H929 at a 1:5 target to effector cell ratio for 3 days. The cytokine release profiles were also measured by MSD ELISA.

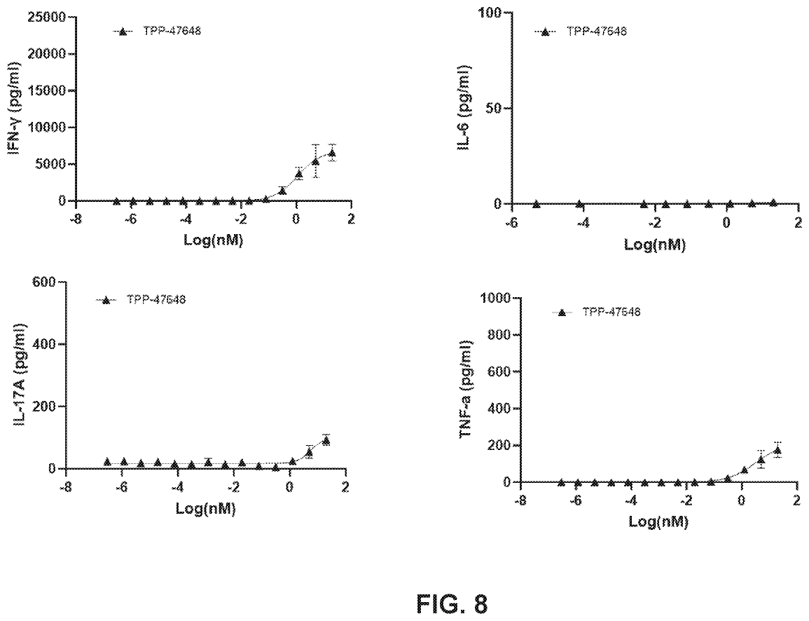

The in vitro cytokine release profile showed that the levels of pro-inflammatory cytokine (TNFα, IL-6, and IL-17a) that was induced by TPP-47648 was minimal, and a dose dependant increase in IFNγ secretion was observed (FIG. 8).

### In Vitro Cytokine Release Profile on B-Cells in Healthy Donor Derived PBMCs

TPP-47648 (TPP48) was assessed for in vitro cytokine release profile on B-cells in healthy donor derived PBMCs. Frozen PBMCs were thawed and cultured in the presence of increasing concentrations (0.00000002 nM to 20 nM) of TPP-47648 for 3 days. The levels of cytokine secretion were determined in the supernatant by MSD ELISA.

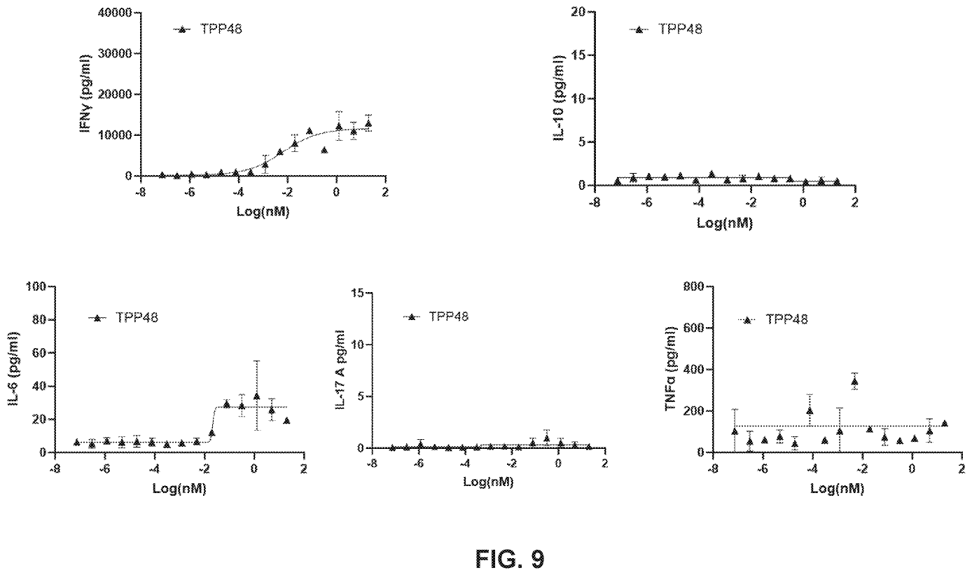

FIG. 9 shows the in vitro cytokine release profiles of TPP-47468. The results show that there was minimal pro-inflammatory cytokine secretion (TNFα, IL-6, IL-10 and IL-17a) and a dose dependant increase in IFN-γ.

### Example 7: In Vivo Analysis of B Cell Depletion by TPP-47468 in a Humanized Mouse Model

The objective of this study was to evaluate whether a single dose of TPP-47468 depletes B-cells in blood and tissues using a humanized mouse model using (NSG)-CD34+ engrafted mice. (NSG)-CD34+ engrafted mice have no detectable plasmablasts or plasma cells in blood and bone marrow and only minimal levels in spleen, making this model unsuitable for assessing the efficacy of TPP-47468 against plasmablasts and plasma cells. Forty-two females NOD-scid-IL2Rgammanull (NSG)-CD34+ engrafted mice obtained from Jackson lab at about 12 weeks post-engraftment were randomly assigned into 7 groups based on body weight and T-cell/B-cell ratio, with 6 mice assigned per group. On day 0 mice were administered intraperitoneally with a single dose of either vehicle (phosphate-buffered saline), 10 mg/kg control antibody LM-1909 (human IgG1 isotype control), or 0.04 mg/kg, 0.15 mg/kg, 0.65 mg/kg 2.5 mg/kg or 10 mg/kg of TPP-47468. On day 3, mice were euthanized, blood and tissues were collected for FACS analysis to determine whether TPP-47468 depleted B cells from blood, spleen and bone marrow. B cells were defined by flow cytometry analysis as human CD45+CD3−CD19+.

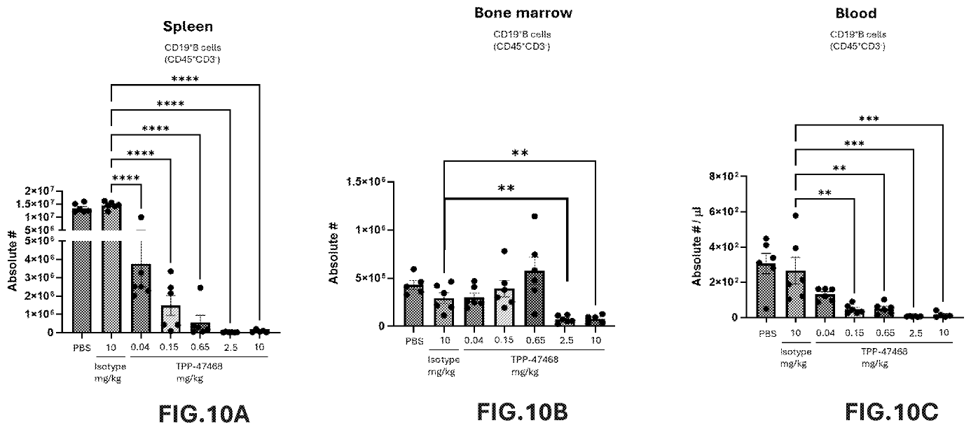

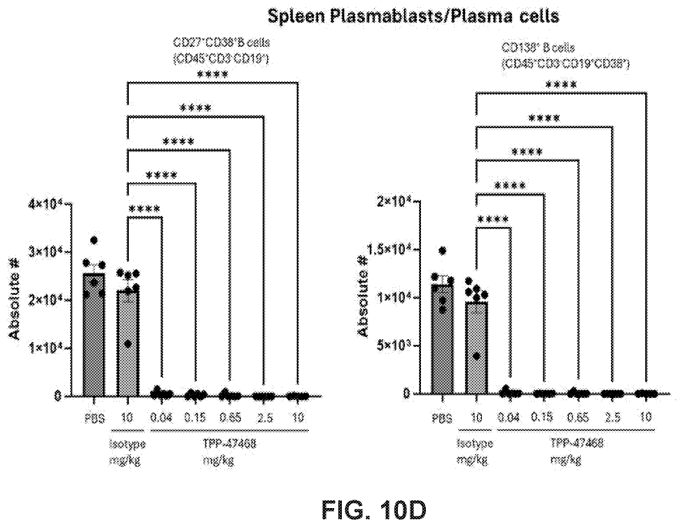

B cells were depleted in mice three days following a single treatment of TPP-47468 in blood and the indicated tissues of the engrafted mice when compared with the activity of its control antibody LM-1909 (isotype control) and vehicle in a dose dependent manner (FIG. 10). The 10 mg/kg and 2.5 mg/kg doses of TPP-47468 demonstrated the highest level of depletion with respect to B-cells in spleen (FIG. 10A), bone marrow (FIG. 10B) and blood (FIG. 10C). No B cell depletion was observed in bone marrow at low dose (FIG. 10B). Very limited numbers of CD27+CD38⁺ plasmablasts (gated in Live⁺ human CD45+CD3−CD19+ B cells) and CD138+ plasma cells (gated in Live⁺ human CD45+CD3−CD19+CD38+) were observed in the spleen (FIG. 10D) which was efficiently depleted following administration of TPP-47468 at the lowest dose (0.04 mg/kg).

### Example 8: In Vivo Analysis of the Depletion of Primary B Cells by TPP-47468 in a XenoGvHD Model

To assess the ability of TPP-47468 to deplete primary B cells expressing BCMA in vivo, a pharmacodynamic (PD) study was performed in the xenogeneic model of graft versus host disease (XenoGvHD). To prepare for this XenoGvHD PD study, healthy human donor peripheral blood mononuclear cells (PBMCs) were isolated from fresh Leukopak (StemExpress) and stored frozen (liquid nitrogen tank) until the engraftment.

On Day −1, 8-10-week-old female NOD scid gamma mice (NSG; Jackson Laboratories) were pre-conditioned with sub-lethal whole-body irradiation (1 Gy). On Day 0, 15 million total PBMC from a healthy human donor was injected intravenously. On day 9, mice received either phosphate buffered saline (PBS), 0.3 mg/kg, 0.1 mg/kg, 0.03 mg/kg, 0.01 mg/kg, 0.003 mg/kg of TPP-47468, or 0.3 mg/kg isotype control, intraperitoneally (n=6 mice/group). On Day 12, mice were euthanized, and tissues were collected for FACS analysis to determine whether these TPP-47468 depleted the BCMA+ cells present in this in vivo model.

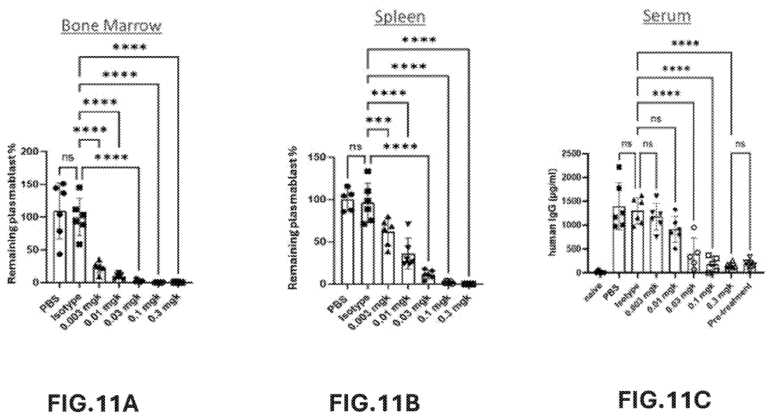

Plasmablasts, defined by flow cytometric analysis, gated as human CD45+CD27+CD38+CD3−CD56−CD14−CD16−, were depleted in mice three days following a single treatment of TPP-47468 in both the bone marrow and in the spleen in a dose-depend manner (FIG. 11A and FIG. 11B). Serum human IgG was significantly lower in the TPP-47468 treatment groups compared to the isotype control treated group (FIG. 11C), reflecting the absence of BCMA+ antibody secreting cells in the TPP-47468 treated mice.

### Example 9: Efficacy of TPP-47651 (CD20:BCMA) and TPP-47648 (BCMA:CD20) in the Cynomolgus Macaque Model

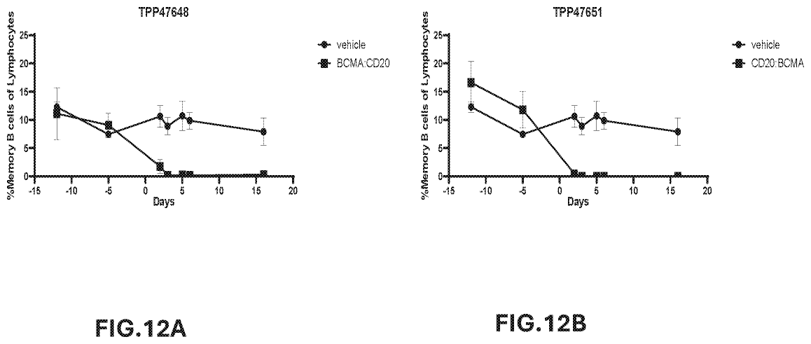

To assess the ability of TPP-47648 to deplete memory B cells in the Cynomolgus Macaque (Cyno), a study was performed in Cyno monkeys that were intravenously dosed with TPP-47648 and TPP-47651. This study was performed by Shin Nippon Biomedical Laboratories, Ltd (study number SBL446-009). This study was approved by the Institutional Animal Care and Use Committee (Approval No. IACUC446-009). Two separate doses of vehicle, 3 mg/kg TPP-47648 (BCMA:CD20) or 3 mg/kg TPP-47651 (CD20:BCMA) were intravenously administered on day 1 and 4. In order to assess the depletion of memory B cells, whole blood was collected on days: −12, −5, 2, 3, 5, 6 and 16. Flow cytometry was performed on lysed whole blood for analysis of memory B cells (CD19+CD27+) and the depletion by TPP-47468 and TPP-47651 is shown as percentage of memory B cells out of total lymphocyte population. In FIG. 12, a decrease in memory B cells is observed after administration of TPP-47648 (FIG. 12A) and TPP-47651 (FIG. 12B) on day 1 compared to the vehicle. The data demonstrates that both TPP-47648 and TPP-47651 exhibits potent memory B cell depletion activity in the cyno monkey. Data represent SEM of memory B cells of blood draws from 3 animals in each treatment group.

### Example 10: In Vivo Anti-Tumour Efficacy in B Cell Model by TPP-47468 and TPP-47651

NOG mice were irradiated with 200cGy 10 days prior to human PBMC engraftment. Three days later, the mice were injected with 1×10\hat{ }6 NCI-H929 cells (a BCMA+ and CD20− isolate) subcutaneously on the left flank. Seven days after tumor engraftment, and weekly thereafter, mice were treated with the 2 mg/kg of TPP-47468 or TPP-47651 or PBS intravenously. Tumor measurements were taken on day 17, 21, 24, 28, 31 and 32 post treatment.

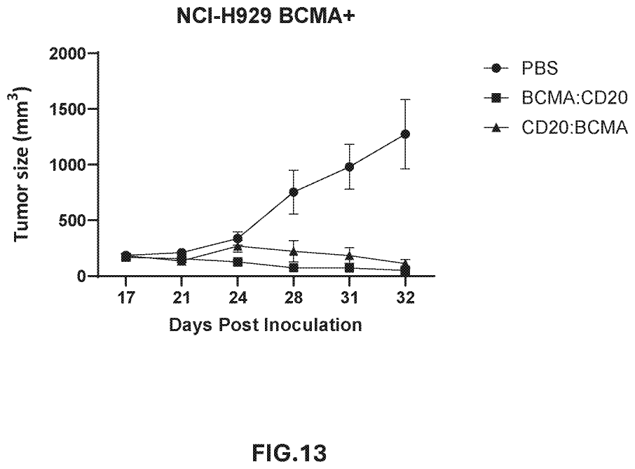

The results demonstrate that TPP-47468 and TPP-47651 were able to reduce the tumor size compared to PBS treatment (FIG. 13). This data shows that both TPP-47468 and TPP-47651 TCE demonstrated anti-tumor efficacy in vivo.

1. (canceled)

2. A binding protein comprising four polypeptide chains that form:
(i) a first tumor-associated antigen (TAA) binding site that binds BCMA;
(ii) a second TAA binding site that binds CD20;
(iii) a T cell receptor (TCR) binding site; and
(iv) a T cell co-stimulatory molecule binding site;
wherein the first polypeptide chain has a structure represented by the formula:
V_{L1}—C_{L1a}
the second polypeptide chain has a structure represented by the formula:
V_{L2}—C_{L1b}
the third polypeptide chain has a structure represented by the formula:
V_{H1}—C_{H1a}—V_{HHa}—Fcₐ
and the fourth polypeptide chain has a structure represented by the formula:
V_{H2}—C_{H1b}—V_{HHb}—Fc_{b};
wherein the V_{L1} domain of the first polypeptide and the V_{H1} domain of the third polypeptide form the BCMA binding site, and the V_{L2} domain of the second polypeptide and the V_{H2} domain of the fourth polypeptide form the CD20 binding site; and
wherein:
V_{L1} is an immunoglobulin light chain variable domain and V_{H1} is an immunoglobulin heavy chain variable domain that together form the BCMA binding site;
V_{L2} is an immunoglobulin light chain variable domain and V_{H2} is an immunoglobulin heavy chain variable domain that together form the CD20 binding site;
C_{L1a} and C_{L1b} are each an immunoglobulin light chain constant domain;
C_{H1a} and C_{H1b} are each an immunoglobulin CH1 heavy chain constant domain;
V_{HHa} is a single chain variable domain that forms the TCR binding site;
V_{HHb} is a single chain variable domain that forms the T cell co-stimulatory molecule binding site;
Fcₐ comprises a C_{H2a} and a C_{H3a} immunoglobulin heavy chain constant domain, wherein the C_{H2a} domain is a first CH2 immunoglobulin heavy chain constant domain and the C_{H3a} domain is a first CH3 immunoglobulin heavy chain constant domain; and;
Fc_{b} comprises a C_{H2b} and a C_{H3b} immunoglobulin heavy chain constant domain wherein the C_{H2b} domain is a second CH2 immunoglobulin heavy chain constant domain and the C_{H3b} domain is a second CH3 immunoglobulin heavy chain constant domain.

3. (canceled)

4. (canceled)

5. The binding protein of claim 2, wherein the binding protein activates T cells only when bound to a tumor associated antigen (TAA) at one or both of the TAA binding sites.

6. The binding protein of claim 2, wherein the V_{HHa} comprises:
(a) a heavy chain CDR1 (HCDR1), a heavy chain CDR2 (HCDR2) and a heavy chain CDR3 (HCDR3), comprising the amino acid sequences of SEQ ID NO: 14, SEQ ID NO: 15 and SEQ ID NO: 16, respectively;
or
(b) a heavy chain CDR1 (HCDR1), a heavy chain CDR2 (HCDR2) and a heavy chain CDR3 (HCDR3) comprising the amino acid sequences of SEQ ID NO: 37, SEQ ID NO: 15 and SEQ ID NO: 16, respectively.

7. The binding protein of claim 2, wherein the VHHa comprises an amino acid sequence that is at least 90%, at least 91% at least 92%, at least 93%, at least 94%, at least 95%, at least 96%, at least 97%, at least 98%, at least 99%, or at least 100% identical to the amino acid sequence of any one of SEQ ID NOs: 17-23; and/or wherein the VHHb comprises an amino acid sequence at least 90%, at least 91% at least 92%, at least 93%, at least 94%, at least 95%, at least 96%, at least 97%, at least 98%, at least 99%, or at least 100% identical to the amino acid sequence of SEQ ID NO: 104.

8. (canceled)

9. (canceled)

10. The binding protein of claim 2, wherein the T cell costimulatory molecule is CD8.

11. The binding protein of claim 2, wherein the V_{HHb} comprises:
(a) a heavy chain CDR1 (HCDR1), a heavy chain CDR2 (HCDR2) and a heavy chain CDR3 (HCDR3) comprising the amino acid sequences of SEQ ID NO: 24, SEQ ID NO: 25 and SEQ ID NO: 26, respectively;
or
(b) a heavy chain CDR1 (HCDR1), a heavy chain CDR2 (HCDR2) and a heavy chain CDR3 (HCDR3) comprising the amino acid sequences of SEQ ID NO: 105, SEQ ID NO: 106 and SEQ ID NO: 107, respectively.

12. (canceled)

13. The binding protein of claim 2, wherein Fcₐ comprises the amino acid sequence of SEQ ID NO: 31 or SEQ ID NO: 32; and/or wherein Fc_{b} comprises the amino acid sequence of SEQ ID NO: 33.

14. (canceled)

15. The binding protein of claim 2, wherein the third polypeptide chain comprises the amino acid sequence of SEQ ID NO: 135, and the fourth polypeptide chain comprises the amino acid sequence of SEQ ID NO: 136.

16. The binding protein of claim 2, wherein the CD20 binding site comprises a heavy chain CDR1 (HCDR1), a heavy chain CDR2 (HCDR2), a heavy chain CDR3 (HCDR3), a light chain CDR1 (LCDR1), a light chain CDR2 (LCDR2) and a light chain CDR3 (LCDR3), comprising the amino acid sequences of SEQ ID NO: 1, SEQ ID NO: 2, SEQ ID NO: 3, SEQ ID NO: 4, SEQ ID NO: 5, and SEQ ID NO: 6, respectively.

17. The binding protein of claim 16, wherein the CD20 binding site comprises a V_{H2} that is at least 90%, at least 91% at least 92%, at least 93%, at least 94%, at least 95%, at least 96%, at least 97%, at least 98%, at least 99% identical, or at least 100% identical to the amino acid sequence of SEQ ID NO: 7; and a V_{L2} that is at least 90%, at least 91% at least 92%, at least 93%, at least 94%, at least 95%, at least 96%, at least 97%, at least 98%, at least 99% identical, or at least 100% identical to the amino acid sequence of SEQ ID NO: 8.

18. The binding protein of claim 2, wherein the BCMA binding site comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3), comprising the amino acid sequences of SEQ ID NO: 76, SEQ ID NO: 77, SEQ ID NO: 78, SEQ ID NO: 79, SEQ ID NO: 80, and SEQ ID NO: 81, respectively.

19. The binding protein of claim 18, wherein the BCMA binding site comprises a V_{H1} that is at least 90%, at least 91% at least 92%, at least 93%, at least 94%, at least 95%, at least 96%, at least 97%, at least 98%, at least 99%, or at least 100% identical to the amino acid sequence of SEQ ID NO: 82; and a V_{L1} domain that is at least 90%, at least 91% at least 92%, at least 93%, at least 94%, at least 95%, at least 96%, at least 97%, at least 98%, at least 99% identical, or at least 100% identical to the amino acid sequence of SEQ ID NO: 83.

20. The binding protein of claim 2 comprising the amino acid sequences of SEQ ID NO: 10, SEQ ID NO: 84, SEQ ID NO: 11, and SEQ ID NO: 86.

21. The binding protein of claim 20, wherein the first polypeptide chain comprises the amino acid sequence of SEQ ID: 84, the second polypeptide chain comprises the amino acid sequence of SEQ ID: 10, the third polypeptide chain comprises the amino acid sequence of SEQ ID: 86, and the fourth polypeptide chain comprises the amino acid sequence of SEQ ID: 11.

22. (canceled)

23. (canceled)

24. A pharmaceutical composition comprising the binding protein of claim 2 and a pharmaceutically acceptable carrier.

25. An isolated nucleic acid molecule encoding any one of the polypeptide chains of the binding protein of claim 2, comprising:
(a) the nucleotide sequence according to any one of SEQ ID NOs: 118-121;
or
(b) the nucleotide sequence according to any one of SEQ ID NOs: 122-125.

26. A vector comprising the isolated nucleic acid molecule of claim 25.

27. (canceled)

28. A method of treating an inflammatory disease and/or autoimmune disorder in a subject in need thereof, comprising administering to the subject a therapeutically effective amount of the binding protein of claim 2.

29. The method of claim 28, wherein;
(i) the binding protein preferentially activates a subset of T cells in the subject, optionally wherein the subset of T cells are CD8+ T cells;
(ii) the CD8+ T cells are preferentially activated as compared to CD4+ T cells:
(iii) the activation of T cells is determined by measuring the percentage of surface CD25+ T cells; and/or
(iv) the percentage of surface CD25+ T cells that are CD8+ T cells is higher than the percentage of surface CD25+ T cells that are CD4+ T cells.

30. (canceled)

31. (canceled)

32. (canceled)

33. The method of claim 28, wherein the binding protein is administered subcutaneously or by intravenously.

34. (canceled)

35. (canceled)

36. (canceled)


SEQUENCE LISTING (138 sequences)

SEQ ID NO: 1 - protein, 5 aa, synthetic construct
SYNMH

SEQ ID NO: 2 - protein, 17 aa, synthetic construct
AIYPGSGDTSYNQKFKG

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 120 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSS

SEQ ID NO: 8 - protein, 106 aa, synthetic construct [sequence omitted]

SEQ ID NO: 9 - protein, 220 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRV

SEQ ID NO: 10 - protein, 212 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKGQPKAAPSVRLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 11 - protein, 591 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSVDKTGGSEVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 4 aa, synthetic construct
TGGS

SEQ ID NO: 13 - protein, 5 aa, synthetic construct
GGGGS

SEQ ID NO: 14 - protein, 5 aa, synthetic construct
INFLG

SEQ ID NO: 15 - protein, 16 aa, synthetic construct
HISIGDQTDYADSAKG

SEQ ID NO: 16 - protein, 9 aa, synthetic construct
FSRIYPYDY

SEQ ID NO: 17 - protein, 117 aa, synthetic construct
EVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSS

SEQ ID NO: 18 - protein, 117 aa, synthetic construct
EVQLLESGGGLVQPGGSLRLSCAASGDVHKINFLSWYRQAPGKEREKVAHISIGDQTDYADSAKGRFTISRDESKNTLYLQMNSLRAEDTAVYYCRAFSRIYPYDYWGQGTLVTVSS

SEQ ID NO: 19 - protein, 117 aa, synthetic construct
EVQLLESGGGLVQPGGSLRLSCAASGDVHKINFLGWYRQAPGKEREKVAHISIGDQTDYADSAKGRFTISRDESKNTLYLQMNSLRAEDTAVYYCRAFSRIYPYDYWGQGTLVTVSS

SEQ ID NO: 20 - protein, 117 aa, synthetic construct
EVQLLESGGGLVQPGGSLRLSCAASGDVHKINFLSWYRQAPGKGLEKVAHISIGDQTDYADSAKGRFTISRDESKNTLYLQMNSLRAEDTAVYYCRAFSRIYPYDYWGQGTLVTVSS

SEQ ID NO: 21 - protein, 117 aa, synthetic construct
EVQLLESGGGLVQPGGSLRLSCAASGDVHKINFLGWYRQAPGKGLEKVAHISIGDQTDYADSAKGRFTISRDESKNTLYLQMNSLRAEDTAVYYCRAFSRIYPYDYWGQGTLVTVSS

SEQ ID NO: 22 - protein, 117 aa, synthetic construct
EVQLLESGGGLVQPGGSLRLSCAASGDVHKINFLGWYRQAPGKEREKVAHISIGDQTDYADSAKGRFTISRDESKNTVYLQMNSLRAEDTAVYFCRAFSRIYPYDYWGQGTLVTVSS

SEQ ID NO: 23 - protein, 117 aa, synthetic construct
EVQLLESGGGLVQPGGSLRLSCAASGDVHKINFLGWYRQAPGKGLEKVAHISIGDQTDYADSAKGRFTISRDESKNTVYLQMNSLRAEDTAVYFCRAFSRIYPYDYWGQGTLVTVSS

SEQ ID NO: 24 - protein, 5 aa, synthetic construct
DYAIG

SEQ ID NO: 25 - protein, 17 aa, synthetic construct
CIRVSDGSTYYADSVKG

SEQ ID NO: 26 - protein, 22 aa, synthetic construct
GSLYTCVQSIVWPARPYYDMDY

SEQ ID NO: 27 - protein, 131 aa, synthetic construct
EVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSS

SEQ ID NO: 28 - protein, 12 aa, synthetic construct
DKTHTCPPCPAP

SEQ ID NO: 29 - skipped

SEQ ID NO: 30 - protein, 10 aa, synthetic construct
THTCPPCPAP

SEQ ID NO: 31 - protein, 214 aa, synthetic construct
PVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 32 - protein, 214 aa, synthetic construct
PVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 33 - protein, 214 aa, synthetic construct
PVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 36 - protein, 469 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 5 aa, synthetic construct
INFLS

SEQ ID NO: 38 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 39 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 41 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 42 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 45 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 46 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLSWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 47 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTLYLQMNSLRAEDTAVYYCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 48 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKEREKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 49 - protein, 455 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGDVHKINFLGWYRQAPGKGLEKVAHISIGDQTDYADSAKGRFTISRDESKNTVYLQMNSLRAEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 50 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGSLYTCVNSIVWPARPYYDMDYWGQGTLVTVSS

SEQ ID NO: 51 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGSAYTCVQSIVWPARPYYDMDYWGQGTLVTVSS

SEQ ID NO: 52 - protein, 131 aa, synthetic construct
EVQLLESGGGLVQPGGSLRLSCAASGFTFRDYAIGWFRQAPGKEREGVSCIRVSDGSTYYADSVKGRFTISRDNAKNTLYLQMNSLRAEDTAVYYCAAGSLYTCVQSIVWPARPYYDMDYWGQGTLVTVSS

SEQ ID NO: 53 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGGLYTCVQSIVWPARPYYDMDYWGQGTLVTVSS

SEQ ID NO: 54 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGSLITCVQSIVWPARPYYDMDYWGQGTLVTVSS

SEQ ID NO: 55 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGPLYTCVQSIVWPARPYYDMDYWGQGTLVTVSS

SEQ ID NO: 56 - protein, 131 aa, synthetic construct
EVQLLESGGGLVQPGGSLRLSCAASGFTFDSYAIGWFRQAPGKEREGVSCIRVSDGSTYYADSVKGRFTISRDNAKNTLYLQMNSLRAEDTAVYYCAAGSLYTCVQSIVWPARPYYDMDYWGQGTLVTVSS

SEQ ID NO: 57 - protein, 131 aa, synthetic construct
EVQLLESGGGLVQPGGSLRLSCAASGFTFPDYAIGWFRQAPGKEREGVSCITVSDGSTYYADSVKGRFTISRDNAKNTLYLQMNSLRAEDTAVYYCAAGSLYTCVQSIVWPARPYYDMDYWGQGTLVTVSS

SEQ ID NO: 58 - protein, 131 aa, synthetic construct
EVQLLESGGGLVQPGGSLRLSCAASGFTFPDYAIGWFRQAPGKEREGVSCIRVSDGSTYYADSVKGRFTISRDNAKNTLYLQMNSLRAEDTAVYYCAAGSLYTCVQSIVWPARPYYDMDYWGQGTLVTVSS

SEQ ID NO: 59 - protein, 7 aa, synthetic construct
EPKSCDK

SEQ ID NO: 60 - protein, 8 aa, synthetic construct
THTCPPCP

SEQ ID NO: 61 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 62 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 63 - protein, 103 aa, synthetic construct
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSV

SEQ ID NO: 64 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 65 - protein, 106 aa, synthetic construct
GQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 66 - protein, 107 aa, synthetic construct [sequence omitted]

SEQ ID NO: 67 - protein, 105 aa, synthetic construct
ELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 68 - protein, 104 aa, synthetic construct
PVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTIS

SEQ ID NO: 69 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 103 aa, synthetic construct
ASTKGPSVFPLAPSSKSTSGGTASLGCLVKDYFPEPVTVSWNSGALTSGVHTFPAVLQSSGLYSLSSVVTVPSSSLGTQTYICNVNHKPSNTKVDKRVEPKSC

SEQ ID NO: 71 - protein, 106 aa, synthetic construct
GQPKAAPSVRLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 72 - protein, 103 aa, synthetic construct
ASTKGPSVCPLAPSSKSTSGGTASLGCLVKDYFPEPVTVSWNSGALTSGVHTFPAVLQSSGLYSLSSVVTVPSSSLGTQTYICNVNHKPSNTKVDKRVEPKSV

SEQ ID NO: 73 - protein, 106 aa, synthetic construct
GQPKAAPSVRLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 74 - protein, 107 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 75 - protein, 106 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTIS

SEQ ID NO: 76 - protein, 5 aa, synthetic construct
SYSMN

SEQ ID NO: 77 - protein, 17 aa, synthetic construct
SISGQSNYIYYADSVKG

SEQ ID NO: 78 - protein, 11 aa, synthetic construct
GGNYFVEYFQY

SEQ ID NO: 79 - protein, 12 aa, synthetic construct
RASQYISSNNLA

SEQ ID NO: 80 - protein, 7 aa, synthetic construct
GASNRAT

SEQ ID NO: 81 - protein, 9 aa, synthetic construct
QQYADSPIT

SEQ ID NO: 82 - protein, 120 aa, synthetic construct; identical to SEQ ID NO: 7

SEQ ID NO: 83 - protein, 108 aa, synthetic construct
EIVLTQSPGTLSLSPGERATLSCRASQYISSNNLAWYQQKPGQAPRLLIYGASNRATGIPDRFSGSGSGTDFTLTISRLEPEDFAVYYCQQYADSPITFGQGTKLEIK

SEQ ID NO: 84 - protein, 215 aa, synthetic construct
EIVLTQSPGTLSLSPGERATLSCRASQYISSNNLAWYQQKPGQAPRLLIYGASNRATGIPDRFSGSGSGTDFTLTISRLEPEDFAVYYCQQYADSPITFGQGTKLEIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 218 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRV

SEQ ID NO: 86 - protein, 575 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 87 - skipped

SEQ ID NO: 88 - protein, 214 aa, synthetic construct
EIVLTQSPGTLSLSPGERATLSCRASQYISSNNLAWYQQKPGQAPRLLIYGASNRATGIPDRFSGSGSGTDFTLTISRLEPEDFAVYYCQQYADSPITFGQGTKLEIKGQPKAAPSVRLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 89 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 589 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSVDKTGGSEVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 577 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 92 - protein, 120 aa, synthetic construct; identical to SEQ ID NO: 7

SEQ ID NO: 93 - protein, 108 aa, synthetic construct; identical to SEQ ID NO: 83

SEQ ID NO: 94 - protein, 215 aa, synthetic construct
EIVLTQSPGTLSLSPGERATLSCRASQYISSNNLAWYQQKPGQAPRLLIYGASNRATGIPDRFSGSGSGTDFTLTISRLEPEDFAVYYCQQYADSPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 223 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 96 - protein, 589 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 575 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 98 - protein, 122 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSS

SEQ ID NO: 99 - protein, 106 aa, synthetic construct [sequence omitted]

SEQ ID NO: 100 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 225 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 102 - protein, 591 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 577 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 104 - protein, 131 aa, synthetic construct
EVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSS

SEQ ID NO: 105 - protein, 8 aa, synthetic construct
GFTFTDYA

SEQ ID NO: 106 - protein, 8 aa, synthetic construct
IRVSDGST

SEQ ID NO: 107 - protein, 24 aa, synthetic construct
AAGSLYTCVQSIVWPARPYYDMDY

SEQ ID NO: 108 - protein, 117 aa, synthetic construct
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSS

SEQ ID NO: 109 - protein, 5 aa, synthetic construct
INFYG

SEQ ID NO: 110 - protein, 16 aa, synthetic construct
HISIGDQTDYADSAKG

SEQ ID NO: 111 - protein, 9 aa, synthetic construct
LSRIWPYDY

SEQ ID NO: 112 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 113 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 114 - protein, 448 aa, synthetic construct
QLOLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 116 - protein, 7 aa, synthetic construct
EPKSVDK

SEQ ID NO: 117 - protein, 17 aa, synthetic construct
CIRVSDGSTYYADSVKG

SEQ ID NO: 118 - DNA, 1773 bp, synthetic construct
  source 1..1773: mol_type = other DNA
gaggtgcagctggtggaatccggtgggggtctggtacagcctgggggttcactgagattgtcatgcgcagcctccgggtacacattcacatcatacaatatgcactgggtgaggcaagctcctggcaaagggttggaatgggtaggagcaatttatcctggcagcggcgacacctcatataaccaaaaatttaaaggtcggtttaccataagtgtcgacaaatccaaaaacaccttgtatttgcagatgaactcactgcgagctgaagacacagcagtctattactgtgctcgtgtagtatattactcaaattcctactggtattttgatgtatgggggcagggcactctcgtgactgtatcaagcgcaagtacaaagggaccttctgtgtgtcctctggctccctccagcaagtctacctctggtggaacagctgacctgggctgcctggtcaaggattactttcctgagcctgtgaccgtgtcctggaactctggcgctctgacatctggcgtgcacacctttccagctgtgctgcagtcctccggcctgtactctctgtcctctgtcgtgaccgtgccttccagctctctgggcacccagacctacatctgcaatgtgaaccacaagccttccaacaccaaggtggacaagagagtggaaccaaaaagcgtggacaagaccggaggatctgaggtacagctgctggaatcaggaggcggcttggttcagccaggaggctcacttcggctctcctgcgcagcaagtgggtttacatttactgattatgctataggctggtttcggcaagcacctggtaaggaacgcgaaggtgtgtcatgtattcgcgtaagcgacggatcaacatactatgcagacagtgtcaaaggtagatttactattagtcgtgacagctcaaagaatactctgtacttgcaaatgaattcccttcgtgccgaagatactgccgtttactattgcgccgcaggatcactctacacctgcgtgcagagcattgtttggcctgccaggccttattatgatatggactattggggccaaggcactttggtcactgttagctctggtggcggaggatctacccacacctgtcctccatgccctgctccacccgtggccggcccatccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcccccatcgaaaagaccatctccaaggccaagggccagcctagggaaccccaggtttacaccctgccaccttgccgggaagagatgaccaagaaccaggtgtccctgtggtgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaa

SEQ ID NO: 119 - DNA, 1725 bp, synthetic construct
  source 1..1725: mol_type = other DNA
gaggtgcagctggtggaaagtggaggtggcctggttaagcctggcgggagcctgcgcctgagctgcgctgccagcggcttcactttctcatcttacagcatgaactgggtgcgccaggcccctggcaaaggccttgagtgggttagtagcatcagcggccagtcaaactacatttattacgccgattccgtcaagggcaggtttacaatctcccgtgataatgcgaagaatagcctctatctccagatgaattccctgcgcgccgaagatacagccgtgtattactgcgcgaggggcgggaattactttgttgaatacttccagtactggggccagggtaccctcgtcaccgtttccagtgcaagtacaaagggaccttctgtgttccctctggctccctccagcaagtctacatctggtggaaccgctgctctgggctgcctggtcaaggattactttcctgagcctgtgaccgtgtcctggaattctggtgctctgacctccggcgtgcacacctttccagctgtgctgcagtcctctggcctgtactctctgaagtctgtcgtgaccgtgccttctagctctctgggcacccagacctacatctgcaacgtgaaccacaagccttccaacaccaaggtggacaagagagtggaaccaaaaagctgcgacaagaccggaggatctgaagtgcagctggtcgagtctggcggaggattggttcaacctggcggctccctgagactgtcttgtgtggctagcggagatgtgcataagatcaattttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtgtcatctggtggcggaggatctacccacacctgtcctccatgccctgctccacccgtggccggcccttccgtgttcctgtttcctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcccccatcgaaaagaccatctctaaggctaagggccagcctcgcgagcctcaagtctgtacactgcctcctagccgggaagagatgaccaagaaccaggtgtccctgtcttgcgccgtgaagggcttctacccttccgatatcgccgtggaatgggagtccaatggccagcctgagaacaactacaagacaacccctcctgtgctggactccgacggctcattcttcctggtgtccaagctgacagtggacaagtccagatggcagcagggcaacgtgttctcctgctctgtgatgcacgaggccctgcacaaccggttcacccagaaatccctgtctctgtcccctggcaag

SEQ ID NO: 120 - DNA, 636 bp, synthetic construct
  source 1..636: mol_type = other DNA
gacattcagatgacacaaagcccatccagccttagcgcatctgtaggagatagagttactataacttgtcgggcctcatctagtgtcagttacatgcactggtaccagcaaaaacccggcaaagcacccaagccacttatctatgctcccagtaaccttgcaagcggcgtacccagccgatttagtggaagtggaagcggaaccgacttcacacttaccatttcctcacttcagcccgaggatttcgccacctactattgccagcagtggagttttaaccctcccacttttggacaaggaacaaaagttgaaataaaaggccaaccgaaggccgctccttccgtgaggctgttccctccatgctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctacagaggtgtcc

SEQ ID NO: 121 - DNA, 645 bp, synthetic construct
  source 1..645: mol_type = other DNA
gagattgtcctgacacagtcccctggcacccttagcctgtcccccggtgaaagagcgaccctgtcctgccgtgcctcacaatacatctcttccaacaatctggcgtggtaccaacagaagcccgggcaagctccccgcctcctgatttacggggcatccaatagagccaccgggatccctgatagattctccggcagcggtagcggtaccgatttcaccctgaccatctctcgcctggagcctgaagacttcgcggtgtactattgtcaacagtatgctgattccccaatcacattcggccagggtactaagcttgaaatcaagcggacagtggccgcaccgtccgtgttcatcttcccaccttccgacgagcagctgaagtctggcacagcctctgtcgagtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 122 - DNA, 1767 bp, synthetic construct
  source 1..1767: mol_type = other DNA
gaggtgcagctggtggaaagtggaggtggcctggttaagcctggcgggagcctgcgcctgagctgcgctgccagcggcttcactttctcatcttacagcatgaactgggtgcgccaggcccctggcaaaggccttgagtgggttagtagcatcagcggccagtcaaactacatttattacgccgattccgtcaagggcaggtttacaatctcccgtgataatgcgaagaatagcctctatctccagatgaattccctgcgcgccgaagatacagccgtgtattactgcgcgaggggcgggaattactttgttgaatacttccagtactggggccagggtaccctcgtcaccgtttccagtgcaagtacaaagggaccttctgtgtgtcctctggctccctccagcaagtctacctctggtggaacagctgacctgggctgcctggtcaaggattactttcctgagcctgtgaccgtgtcctggaactctggcgctctgacatctggcgtgcacacctttccagctgtgctgcagtcctccggcctgtactctctgtcctctgtcgtgaccgtgccttccagctctctgggcacccagacctacatctgcaatgtgaaccacaagccttccaacaccaaggtggacaagagagtggaaccaaaaagcgtggacaagaccggaggatctgaggtacagctgctggaatcaggaggcggcttggttcagccaggaggctcacttcggctctcctgcgcagcaagtgggtttacatttactgattatgctataggctggtttcggcaagcacctggtaaggaacgcgaaggtgtgtcatgtattcgcgtaagcgacggatcaacatactatgcagacagtgtcaaaggtagatttactattagtcgtgacagctcaaagaatactctgtacttgcaaatgaattcccttcgtgccgaagatactgccgtttactattgcgccgcaggatcactctacacctgcgtgcagagcattgtttggcctgccaggccttattatgatatggactattggggccaaggcactttggtcactgttagctctggtggcggaggatctacccacacctgtcctccatgccctgctccacccgtggccggcccatccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcccccatcgaaaagaccatctccaaggccaagggccagcctagggaaccccaggtttacaccctgccaccttgccgggaagagatgaccaagaaccaggtgtccctgtggtgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaa

SEQ ID NO: 123 - DNA, 1731 bp, synthetic construct
  source 1..1731: mol_type = other DNA
gaggtgcagctggtggaatccggtgggggtctggtacagcctgggggttcactgagattgtcatgcgcagcctccgggtacacattcacatcatacaatatgcactgggtgaggcaagctcctggcaaagggttggaatgggtaggagcaatttatcctggcagcggcgacacctcatataaccaaaaatttaaaggtcggtttaccataagtgtcgacaaatccaaaaacaccttgtatttgcagatgaactcactgcgagctgaagacacagcagtctattactgtgctcgtgtagtatattactcaaattcctactggtattttgatgtatgggggcagggcactctcgtgactgtatcaagcgcaagtacaaagggaccttctgtgttccctctggctccctccagcaagtctacatctggtggaaccgctgctctgggctgcctggtcaaggattactttcctgagcctgtgaccgtgtcctggaattctggtgctctgacctccggcgtgcacacctttccagctgtgctgcagtcctctggcctgtactctctgaagtctgtcgtgaccgtgccttctagctctctgggcacccagacctacatctgcaacgtgaaccacaagccttccaacaccaaggtggacaagagagtggaaccaaaaagctgcgacaagaccggaggatctgaagtgcagctggtcgagtctggcggaggattggttcaacctggcggctccctgagactgtcttgtgtggctagcggagatgtgcataagatcaattttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtgtcatctggtggcggaggatctacccacacctgtcctccatgccctgctccacccgtggccggcccttccgtgttcctgtttcctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcccccatcgaaaagaccatctctaaggctaagggccagcctcgcgagcctcaagtctgtacactgcctcctagccgggaagagatgaccaagaaccaggtgtccctgtcttgcgccgtgaagggcttctacccttccgatatcgccgtggaatgggagtccaatggccagcctgagaacaactacaagacaacccctcctgtgctggactccgacggctcattcttcctggtgtccaagctgacagtggacaagtccagatggcagcagggcaacgtgttctcctgctctgtgatgcacgaggccctgcacaaccggttcacccagaaatccctgtctctgtcccctggcaag

SEQ ID NO: 124 - DNA, 642 bp, synthetic construct
  source 1..642: mol_type = other DNA
gagattgtcctgacacagtcccctggcacccttagcctgtcccccggtgaaagagcgaccctgtcctgccgtgcctcacaatacatctcttccaacaatctggcgtggtaccaacagaagcccgggcaagctccccgcctcctgatttacggggcatccaatagagccaccgggatccctgatagattctccggcagcggtagcggtaccgatttcaccctgaccatctctcgcctggagcctgaagacttcgcggtgtactattgtcaacagtatgctgattccccaatcacattcggccagggtactaagcttgaaatcaagggccaaccgaaggccgctccttccgtgaggctgttccctccatgctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctacagaggtgtcc

SEQ ID NO: 125 - DNA, 639 bp, synthetic construct
  source 1..639: mol_type = other DNA
gacattcagatgacacaaagcccatccagccttagcgcatctgtaggagatagagttactataacttgtcgggcctcatctagtgtcagttacatgcactggtaccagcaaaaacccggcaaagcacccaagccacttatctatgctcccagtaaccttgcaagcggcgtacccagccgatttagtggaagtggaagcggaaccgacttcacacttaccatttcctcacttcagcccgaggatttcgccacctactattgccagcagtggagttttaaccctcccacttttggacaaggaacaaaagttgaaataaaacggacagtggccgcaccgtccgtgttcatcttcccaccttccgacgagcagctgaagtctggcacagcctctgtcgagtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 126 - DNA, 1773 bp, synthetic construct
  source 1..1773: mol_type = other DNA
gaggtgcagctggtggaatccggtgggggtctggtacagcctgggggttcactgagattgtcatgcgcagcctccgggtacacattcacatcatacaatatgcactgggtgaggcaagctcctggcaaagggttggaatgggtaggagcaatttatcctggcagcggcgacacctcatataaccaaaaatttaaaggtcggtttaccataagtgtcgacaaatccaaaaacaccttgtatttgcagatgaactcactgcgagctgaagacacagcagtctattactgtgctcgtgtagtatattactcaaattcctactggtattttgatgtatgggggcagggcactctcgtgactgtatcaagcgcaagcaccaagggaccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtacagctgctggaatcaggaggcggcttggttcagccaggaggctcacttcggctctcctgcgcagcaagtgggtttacatttactgattatgctataggctggtttcggcaagcacctggtaaggaacgcgaaggtgtgtcatgtattcgcgtaagcgacggatcaacatactatgcagacagtgtcaaaggtagatttactattagtcgtgacagctcaaagaatactctgtacttgcaaatgaattcccttcgtgccgaagatactgccgtttactattgcgccgcaggatcactctacacctgcgtgcagagcattgtttggcctgccaggccttattatgatatggactattggggccaaggcactttggtcactgttagctctggaggcggaggcagcacccatacctgtcctccatgtcctgctcctccagtggctggcccttccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggacagcccagggaaccccaggtttacaccctgcctccatgccgggaagagatgaccaagaaccaggtgtccctgtggtgcctggttaagggcttctacccctccgatatcgccgtggaatgggagtctaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgagccccggcaaa

SEQ ID NO: 127 - DNA, 1731 bp, synthetic construct
  source 1..1731: mol_type = other DNA
gaggtgcagctggtggaatccggtgggggtctggtacagcctgggggttcactgagattgtcatgcgcagcctccgggtacacattcacatcatacaatatgcactgggtgaggcaagctcctggcaaagggttggaatgggtaggagcaatttatcctggcagcggcgacacctcatataaccaaaaatttaaaggtcggtttaccataagtgtcgacaaatccaaaaacaccttgtatttgcagatgaactcactgcgagctgaagacacagcagtctattactgtgctcgtgtagtatattactcaaattcctactggtattttgatgtatgggggcagggcactctcgtgactgtatcaagcgcaagtactaagggcccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtgcagctggtggaatctggcggaggattggttcagcctggcggctctctgagactgtcttgtgtggctagcggcgacgtgcacaagatcaactttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtttctagcggcggaggcggctctacccatacctgtcctccatgtcctgctcctccagtggctggcccatccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaacaggttcacccagaagtccctgtctctgagccctggcaag

SEQ ID NO: 128 - DNA, 639 bp, synthetic construct
  source 1..639: mol_type = other DNA
gacattcagatgacacaaagcccatccagccttagcgcatctgtaggagatagagttactataacttgtcgggcctcatctagtgtcagttacatgcactggtaccagcaaaaacccggcaaagcacccaagccacttatctatgctcccagtaaccttgcaagcggcgtacccagccgatttagtggaagtggaagcggaaccgacttcacacttaccatttcctcacttcagcccgaggatttcgccacctactattgccagcagtggagttttaaccctcccacttttggacaaggaacaaaagttgaaataaaacggacagtggccgcaccgtccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 129 - DNA, 1767 bp, synthetic construct
  source 1..1767: mol_type = other DNA
gaggtgcagctggtggaaagtggaggtggcctggttaagcctggcgggagcctgcgcctgagctgcgctgccagcggcttcactttctcatcttacagcatgaactgggtgcgccaggcccctggcaaaggccttgagtgggttagtagcatcagcggccagtcaaactacatttattacgccgattccgtcaagggcaggtttacaatctcccgtgataatgcgaagaatagcctctatctccagatgaattccctgcgcgccgaagatacagccgtgtattactgcgcgaggggcgggaattactttgttgaatacttccagtactggggccagggtaccctcgtcaccgtttccagtgcaagcaccaagggaccctctgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgataagaccggcggatctgaggtacagctgctggaatcaggaggcggcttggttcagccaggaggctcacttcggctctcctgcgcagcaagtgggtttacatttactgattatgctataggctggtttcggcaagcacctggtaaggaacgcgaaggtgtgtcatgtattcgcgtaagcgacggatcaacatactatgcagacagtgtcaaaggtagatttactattagtcgtgacagctcaaagaatactctgtacttgcaaatgaattcccttcgtgccgaagatactgccgtttactattgcgccgcaggatcactctacacctgcgtgcagagcattgtttggcctgccaggccttattatgatatggactattggggccaaggcactttggtcactgttagctctggaggcggaggcagcacccatacctgtcctccatgtcctgctcctccagtggctggcccttccgtgtttctgttccctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggacagcccagggaaccccaggtttacaccctgcctccatgccgggaagagatgaccaagaaccaggtgtccctgtggtgcctggttaagggcttctacccctccgatatcgccgtggaatgggagtctaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgagccccggcaaa

SEQ ID NO: 130 - DNA, 1725 bp, synthetic construct
  source 1..1725: mol_type = other DNA
gaggtgcagctggtggaaagtggaggtggcctggttaagcctggcgggagcctgcgcctgagctgcgctgccagcggcttcactttctcatcttacagcatgaactgggtgcgccaggcccctggcaaaggccttgagtgggttagtagcatcagcggccagtcaaactacatttattacgccgattccgtcaagggcaggtttacaatctcccgtgataatgcgaagaatagcctctatctccagatgaattccctgcgcgccgaagatacagccgtgtattactgcgcgaggggcgggaattactttgttgaatacttccagtactggggccagggtaccctcgtcaccgtttccagtgcaagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgaaccaaaaagctgcgacaagaccggaggatctgaagtgcagctggtcgagtctggcggaggattggttcaacctggcggctccctgagactgtcttgtgtggctagcggagatgtgcataagatcaattttctcggctggtacagacaggcccctggcaaagagagagagaaggtcgcccacatctccatcggcgaccagaccgattacgccgactctgccaagggcagattcaccatctctcgggacgagtccaagaacatggtgtacctgcagatgaactccctgaagcctgaggataccgccgtgtacttctgccgggccttctctcggatctacccctacgattattggggccagggcaccctggtcacagtgtcatctggtggcggaggatctacccacacctgtcctccatgccctgctccacccgtggccggcccttccgtgttcctgtttcctccaaagcctaaggacaccctgatgatctctcggacccctgaagtgacctgcgtggtggtggatgtgaagcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcccccatcgaaaagaccatctctaaggctaagggccagcctcgcgagcctcaagtctgtacactgcctcctagccgggaagagatgaccaagaaccaggtgtccctgtcttgcgccgtgaagggcttctacccttccgatatcgccgtggaatgggagtccaatggccagcctgagaacaactacaagacaacccctcctgtgctggactccgacggctcattcttcctggtgtccaagctgacagtggacaagtccagatggcagcagggcaacgtgttctcctgctctgtgatgcacgaggccctgcacaaccggttcacccagaaatccctgtctctgtcccctggcaag

SEQ ID NO: 131 - DNA, 645 bp, synthetic construct
  source 1..645: mol_type = other DNA
gagattgtcctgacacagtcccctggcacccttagcctgtcccccggtgaaagagcgaccctgtcctgccgtgcctcacaatacatctcttccaacaatctggcgtggtaccaacagaagcccgggcaagctccccgcctcctgatttacggggcatccaatagagccaccgggatccctgatagattctccggcagcggtagcggtaccgatttcaccctgaccatctctcgcctggagcctgaagacttcgcggtgtactattgtcaacagtatgctgattccccaatcacattcggccagggtactaagcttgaaatcaagcggacagtggccgcaccgtccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 132 - protein, 103 aa, synthetic construct
ASTKGPSVFPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSC

SEQ ID NO: 133 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 134 - protein, 109 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAK

SEQ ID NO: 135 - protein, 455 aa, synthetic construct
ASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 136 - protein, 469 aa, synthetic construct
ASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSVDKTGGSEVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 577 aa, synthetic construct
EVQLVESGGGLVQPGGSLRLSCAASGYTFTSYNMHWVRQAPGKGLEWVGAIYPGSGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 138 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK